(19) United States
(12) Patent Application Publication
(10) Pub. No.: US 2007/0238727 A1
(43) Pub. Date: Oct. 11, 2007

(54) 5-Substituted 1-Phenyl-1,5-Dihydro-Pyrido[3,2-B]Indol-2-Ones and Analogs as Anti-Virals

(75) Inventors: Bart Rudolf Romanie Kesteleyn, Berlare (BE); Maxime Francis Jean-Marie Ghislain Canard, Rosieres (BE); Wim Van De Vreken, Beveren (BE); Pierre Jean-Marie Bernard Raboisson, Sterrebeek (BE); Dominique Louis Nestor Ghislain Surleraux, Braine-le-chateau (BE); Piet Tom Bert Paul Wigerinck, Terhagen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Tibotec BVBA, B-2800 Mechelen (BE)

(21) Appl. No.: 11/568,838

(22) PCT Filed: May 17, 2005
(86) PCT No.: PCT/EP05/52262

§ 371 Date: Nov. 8, 2006

(30) Foreign Application Priority Data
May 17, 2004 (EP) 04102169.2

(51) Int. Cl. A61K 31/4355; A61K 31/496; A61K 31/5377; A61K 31/541; A61P 31/12; C07D 401/14; C07D 413/14; C07D 417/14; C07D 491/044

(52) U.S. Cl. 514/228.2; 514/232.8; 514/253.03; 514/291; 544/126; 544/362; 544/60; 546/89

(57) ABSTRACT

Compounds of formula (I) the N-oxides, salts, stereoisomeric forms, prodrugs, esters and metabolites thereof, wherein X is NR², O, S, SO, SO₂; R¹ is hydrogen, cyano, halo, a carbonyl derivative, methanimidamidyl, N-hydroxy-methanimidamidyl, mono- or di(C₁₋₄alkyl)-methanimidamidyl, Het₁ or Het₂; n is 1, 2 or 3; R² is (i) aryl substituted with a radical —COOR⁴; (ii) C₁₋₁₀alkyl, C₂₋₁₀alkenyl, C₃₋₇cycloalkyl, substituted with aryl which is substituted with a radical —COOR⁴; (iii) C₁₋₁₀alkyl, C₂₋₁₀alkenyl, C₃₋₇cycloalkyl, substituted with —NR^{5a}—C(═NR^{5b})—NR^{5c}R^{5d}, —O—NR^{5a}—C(═NR^{5b})—NR^{5c}R^{5d}, -sulfonyl-R⁶, —NR⁷R⁸, —NR⁹R¹⁰, a radical (a-1), (a-2), (a-3), (a-4), (a-5); or (iv) a radical of formula: (a-6), (b-2), —CₚH₂ₚ—CH(OR¹⁴)—C_{q}H_{2q}—R¹⁵; —CH₂—CH₂—(O—CH₂—CH₂)ₘ—OR¹⁴; —CH₂—CH₂—(O—CH₂—CH2)ₘ—NR^{17a}R^{17b}; R³ is nitro, cyano, amino, halo, hydroxy, C₁₋₄alkyloxy, a carbonyl derivative, methanimidamidyl, mono- or di(C₁₋₄alkyl)methanimidamidyl, N-hydroxy-methanimidamidyl or Het₁.

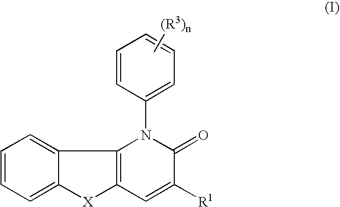

This invention relates to 5-substituted 1-phenyl-1,5-dihydro-pyrido[3,2-b]indol-2-ones, the analogous 1-phenyl-1H-benzo[4,5]furo[3,2-b]pyridine-2-ones and 1-phenyl-1H-benzo[4,5]thieno[3,2-b]pyridine-2-ones, the use of these compounds as HIV inhibitors, to pharmaceutical compositions containing these compounds and to processes for preparing these compounds and compositions.

The virus causing the acquired immunodeficiency syndrome (AIDS) is known by different names, including T-lymphocyte virus III (HTLV-III), lymphadenopathy-associated virus (LAV), AIDS-related virus (ARV) or human immunodeficiency virus (HIV). Up until now, two distinct classes have been identified, i.e. HIV-1 and HIV-2. Hereinafter, the term HIV will be used to generically denote both these classes.

HIV infected patients are currently treated with HIV protease inhibitors (PIs), nucleoside reverse transcriptase inhibitors (NRTIs), non-nucleoside reverse transcriptase inhibitors (NNRTIs) and nucleotide reverse transcriptase inhibitors (NtRTIs). Despite the fact that these antiretrovirals are very useful, they have a common limitation, namely, the targeted enzymes in the HIV virus are able to mutate in such a way that the known drugs become less effective, or even ineffective against these mutant HIV viruses. Or, in other words, the HIV virus creates an ever-increasing resistance against any available drugs, which is a major cause of therapy failure. Moreover, it has been shown that resistant virus is carried over to newly infected individuals, resulting in severely limited therapy options for these drug-naive patients.

Current HIV therapy comprises in most cases the administration of drug cocktails comprising two or more active ingredients selected from the above classes of HIV inhibitors. But even when using combination therapy, drug resistance arises resulting in the combination becoming less effective. This often may force the treating physician to boost the plasma levels of the active drugs in order for said antiretrovirals to regain effectivity against the mutated HIV viruses, the consequence of which is an undesirable increase in pill burden. The latter in turn may also lead to an increased risk of non-compliance with the prescribed therapy.

Therefore, there is a continuous general need for new combinations of HIV inhibitors that comprise new types of HIV inhibitory agents. Hence there is a need for new HIV inhibitors that differ from existing inhibitors in terms of chemical structure as well as mode of action or both. There is a particular need for compounds that are active not only against wild type HIV virus, but also against the increasingly more common resistant HIV viruses.

Currently used HIV reverse transcriptase inhibitors belong to three different classes. These include the NRTIs, which are intracellularly converted to nucleoside triphosphates that compete with the natural nucleoside triphosphates for incorporation into elongating viral DNA by reverse transcriptase. Chemical modifications that distinguish these compounds from natural nucleosides result in DNA chain termination events. NRTIs that are currently available include zidovudine (AZT), didanosine (ddI), zalcitabine (ddC), stavudine (d4T), lamivudine (3TC) and abacavir (ABC). A second class comprises the NtRTIs such as tenofovir, which have a similar mode of action as the NRTIs. Emergence of mutations causes the NRTIs and NtRTIs to become ineffective. A third class comprises the NNRTIs, which interact with the NNRTI binding site and thereby block the RT mechanism. Currently available NNRTIs include nevirapine, delavirdine and efavirenz, known to be susceptible to relative rapid emergence of resistance due to mutations at amino acids that surround the NNRTI-binding site.

Thus, there is a medical need for further anti-infective compounds that target HIV reverse transcriptase, in particular anti-retroviral compounds that are able to delay the occurrence of resistance and that combat a broad spectrum of mutants of the HIV virus.

WO02/055520 and WO02/059123 disclose benzoylalkylindolepyridinium compounds as antiviral compounds. Ryabova et al. disclose the synthesis of certain benzoylalkylindolepyridinium compounds (Russian Chem. Bull. 2001, 50(8), 1449-1456; and Chem. Heterocycl. Compd. (Engl. Translat.) 36; 3; 2000; 301-306; Khim. Geterotsikl. Soedin.; RU; 3; 2000; 362-367).

The compounds of this invention differ from these prior art compounds in terms of chemical structure as well as by the fact that they interact via a mechanism that differs from known RT inhibitors. They not only are active against wild type HIV virus but also against mutant HIV viruses, in particular mutant HIV viruses exhibiting resistance against currently available reverse transcriptase (RT) inhibitors.

Thus in one aspect the present invention concerns 1-phenyl-1,5-dihydro-pyrido[3,2-b]indol-2-ones and analogs thereof which can be represented by formula (I): 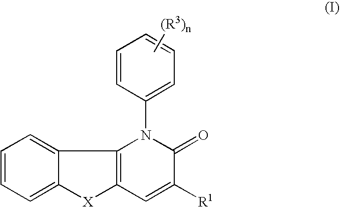
the N-oxides, salts, quaternary ammonium salts, stereoisomeric forms, prodrugs, esters and metabolites thereof wherein
X is a bivalent radical NR², O, S, SO, SO₂;
R¹ is hydrogen, cyano, halo, aminocarbonyl, hydroxycarbonyl, C₁₋₄alkyloxycarbonyl, C₁₋₄alkylcarbonyl, mono- or di(C₁₋₄alkyl)aminocarbonyl, arylaminocarbonyl, N-(aryl)-N—(C₁₋₄alkyl)aminocarbonyl, methanimidamidyl, N-hydroxy-methamidamidyl, mono- or di(C₁₋₄alkyl)methanimidamidyl, Het₁ or Het₂;
n is 1, 2 or 3;
R² is:
i) aryl substituted with a radical —COOR⁴; or R² is
ii) C₁₋₁₀alkyl, C₂₋₁₀-alkenyl, C₃₋₇cycloalkyl,
each of said C₁₋₁₀alkyl, C₂₋₁₀alkenyl, C₃₋₇cycloalkyl, each individually and independently, being substituted with aryl wherein said aryl is substituted with a radical COOR⁴; or R² is
iii) C₁₋₁₀alkyl, C₂₋₁₀alkenyl, C₃₋₇cycloalkyl, each individually and independently, substituted with a radical selected from —NR^{5a}—C(═NR^{5e})—NR^{5c}R^{5d}, —NR^{5a}—C(═NR^{5c})—R^{5f}, —O—NR^{5a}—C(═NR^{5b})—NR^{5c}R^{5d}, —O—NR^{5a}—C(═NR^{5e})—R^{5f}, -sulfonyl-R⁶, —NR⁷R⁸, —NR⁹R¹⁰, a radical 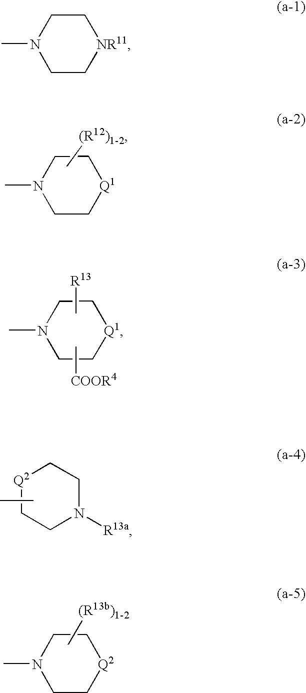
   wherein
each Q¹ independently is a direct bond, —CH₂—, or —CH₂—CH₂—;
each Q² independently is O, S, SO or SO₂;
each R⁴ independently is hydrogen, C₁₋₄alkyl, arylC₁₋₄alkyl;
each R^{5a}, R^{5b}, R^{5c}, R^{5d} independently is hydrogen, C₁₋₄alkyl or arylC₁₋₄alkyl;
each R^{5e}, R^{5f} independently is hydrogen, C₁₋₄alkyl or arylC₁₋₄alkyl, or R^{5e} and R^{5f} taken together may form a bivalent alkanediyl radical of formula —CH₂—CH₂— or —CH₂—CH₂—CH₂—;
R⁶ is C₁₋₄alkyl, —N(R^{5a}R^{5b}), C₁₋₄alkyloxy, pyrrolidin-1-yl, piperidin-1-yl, homopiperidin-1-yl, piperazin-1-yl, 4-C₁₋₄alkyl)-piperazin-1-yl, morpholin-4-yl-, thiomorpholinyl-4-yl-, 1-oxothiomorpholinyl and 1,1-dioxo-thiomorpholin-4-yl;
R⁷ is hydrogen, C₁₋₄alkyl, hydroxyC₁₋₄alkyl, C₁₋₄alkoxyC₁₋₄alkyl or C₁₋₄alkylcarbonyloxyC₁₋₄alkyl;
R⁸ is hydroxyC₁₋₄alkyl, C₁₋₄alkoxyC₁₋₄alkyl, C₁₋₄alkylcarbonyloxyC₁₋₄alkyl, aryl or arylC₁₋₄alkyl;
R⁹ is hydrogen or C₁₋₄alkyl;
R¹⁰ is Het₁, Het₂ or a radical 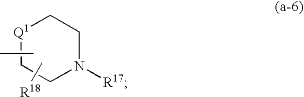
R¹¹ is aryl, arylC₁₋₄alkyl, formyl, C₁₋₄alkylcarbonyl, arylcarbonyl, arylC₁₋₄alkylcarbonyl, C₁₋₄alkyloxycarbonyl, arylC₁₋₄alkyloxycarbonyl, R^{5a}R^{5b}N-carbonyl hydroxyC₁₋₄alkyl, C₁₋₄alkyloxyC₁₋₄alkyl, arylC₁₋₄alkyloxyC₁₋₄alkyl, aryloxyC₁₋₄alkyl, Het₂;
each R¹² independently is hydroxy, C₁₋₄alkyl, arylC₁₋₄alkyl, C₁₋₄alkyloxy, arylC₁₋₄alkyloxy, oxo, spiro(C₂₋₄alkanedioxy), spiro(diC₁₋₄alkyloxy), —NR^{5a}R^{5b};
R¹³ is hydrogen, hydroxy, C₁₋₄alkyl, C₁₋₄alkyloxy, or arylC₁₋₄alkyloxy; or
R^{13a} is C₁₋₄alkyl, arylC₁₋₄alkyl, C₁₋₄alkyloxycarbonyl or arylC₁₋₄alkyloxycarbonyl;
each R^{13b} is hydrogen or C₁₋₄alkyl; or R² is
iv) a radical of formula: 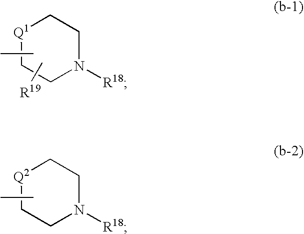 —Cₚ—H₂ₚ—CH(OR¹⁴)—C_{q}H_{2q}—R¹⁵ (b-3);
   —CH₂—CH₂—(O—CH₂—CH₂)ₘ—OR¹⁴ (b-4);
   —CH₂—CH₂—(O—CH₂—CH₂)ₘ—NR^{17a}R^{17b} (b-5);
   wherein in radical (b-3) one of the hydrogen atoms in —CₚH₂ₚ— and one of the hydrogen atoms in —H(OR¹⁴)—C_{q}H_{2q}—, that is not part of R¹⁴, may be replaced by a direct bond or a C₁₋₄alkanediyl group;
p is 1, 2 or 3;
q is 0, 1, 2 or 3;
each m independently is 1 to 10;
each R¹⁴ independently is hydrogen, C₁₋₄alkyl, aryl C₁₋₄alkyl, aryl, C₁₋₄alkylcarbonyl, —SO₃H, —PO₃H₂;
R¹⁵ is a substituent selected from the group consisting of cyano, NR^{16a}R^{16b}, pyrrolidinyl, piperidinyl, homopiperidinyl, piperazinyl, 4-(C₁₋₄alkyl)-piperazinyl, 4-(C₁₋₄alkylcarbonyl)-piperazinyl, 4-(C₁₋₄alkyloxycarbonyl)-piperazinyl, morpholinyl, thiomorpholinyl, 1-oxothiomorpholinyl, 1,1-dioxo-thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl, pyridazinyl, triazinyl, hydroxy-carbonyl, C₁₋₄alkylcarbonyl, N(R^{16a}R^{16b})carbonyl, C₁₋₄alkyloxycarbonyl, pyrrolidin-1-yl-carbonyl, piperidin-1-ylcarbonyl, homopiperidin-1-ylcarbonyl, piperidin-1-yl-carbonyl, 4-(C₁₋₄alkyl)-piperazin-1-ylcarbonyl, morpholin-1-yl-carbonyl, thiomorpholin-1-yl-carbonyl, 1-oxothiomorpholin-1-ylcarbonyl and 1,1-dioxo-thiomorpholin-1-ylcarbonyl; or R¹⁵ may additionally be aryl substituted with a radical —COOR⁴; or a radical selected from —NR^{5a}—C(═NR^{5b})—NR^{5e}R^{5d}, —NR^{5a}—C(═NR^{5e})—R^{5f}, —O—NR^{5a}—C(═NR^{5b})—NR^{5c}R^{5d}, —NR^{5a}—C(═NR^{5e})—R^{5f}-sulfonyl-R⁶, —NR⁷R⁵, —NR⁹R¹⁰, a radical (a-1), (a-2), (a-3), (a-4) or (a-5); wherein R⁴, R^{5a}, e, R^{5c}, R^{5d}, R⁶, R⁷, R⁸, R⁹, R¹⁰, and the radicals (a-1), (a-2), (a-3), (a-4), (a-5) independently are as defined above;
R^{16a} and R^{16b} independently from one another are hydrogen, C₁₋₆alkyl or C₁₋₆alkyl substituted with a substituent selected from the group consisting of amino, mono- or di(C₁₋₄alkyl)amino, pyrrolidinyl piperidinyl, homopiperidinyl, piperazinyl, 4-(C₁₋₄alkyl)-piperarinyl morpholinyl, thiomorpholinyl, 1-oxothiomorpholinyl, 1,1-dioxo-thiomorpholinyl and aryl;
R^{17a} and R^{17b} independently from one another are hydrogen, C₁₋₄alkyl or arylC₁₋₄alkyl;
or R^{17a} and R^{17b} together with the nitrogen atom to which they are attached form a pyrrolidinyl piperidinyl, homopiperidinyl, morpholinyl, thiomorpholinyl, 1-oxothiomorpholinyl, 1,1-dioxo-thiomorpholinyl piperazinyl 4-C₁₋₄alkyl-piperazinyl 4-(C₁₋₄alkylcarbonyl)-piperazinyl, 4-(C₁₋₄alkyloxycarbonyl)-piperazinyl ring;
each R¹⁸ independently is hydrogen, C₁₋₄alkyl, arylC₁₋₄alkyl C₁₋₄alkylcarbonyl or C₁₋₄alkyloxycarbonyl;
R¹⁹ is hydrogen, hydroxy, C₁₋₄alkyl or a radical —COOR⁴;
R³ is nitro, cyano, amino, halo, hydroxy, C₁₋₄alkyloxy, hydroxycarbonyl, aminocarbonyl, C₁₋₄alkyloxycarbonyl, mono- or di(C₁₋₄alkyl)aminocarbonyl, C₁₋₄alkylcarbonyl, methanimidamidyl, mono- or di(C₁₋₄alkyl)methanimidamidyl, N-hydroxy-methanimidamidyl or Het₁;
aryl is phenyl optionally substituted with one or more substituents each individually selected from the group consisting of C₁₋₆alkyl, C₁₋₄alkoxy, halo, hydroxy, amino, trifluoromethyl, cyano, nitro, hydroxyC₁₋₆alkyl, cyanoC₁₋₆alkyl, mono- or di(C₁₋₄alkyl)amino, aminoC₁₋₄alkyl mono- or di(C₁₋₄alkyl)aminoC₁₋₄alkyl;
Het₁ is a 5-membered ring system wherein one, two, three or four ring members are heteroatoms each individually and independently selected from the group consisting of nitrogen, oxygen and sulfur, and wherein the remaining ring members are carbon atoms; and, where possible, any nitrogen ring member may optionally be substituted with C₁₋₄alkyl; any ring carbon atom may, each individually and independently, optionally be substituted with a substituent selected from the group consisting of C₁₋₄alkyl C₂₋₆alkenyl C₃₋₇cycloalkyl hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl mono- or di(C₁₋₄alkyl)aminoC₂₋₆alkenyl furanyl, thienyl pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl C₁₋₄alkyloxycarbonyl mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl;
Het₂ is pyridyl, pyrimidinyl, pyrazinyl, pyridazinyl or triazinyl, wherein any ring carbon atom of each of said 6-membered nitrogen containing aromatic rings may optionally be substituted with C₁₋₄alkyl.

In a particular aspect this invention concerns compounds of formula (I) wherein R₁ is cyano; X is O or NR² wherein R² is a C₁₋₁₀alkyl radical substituted as specified above or R² is a linear radical of formula (b-3) or (b-4); n is 1 and R₃ is nitro.

As used herein “C₁₋₄alkyl” as a group or part of a group defines straight or branched chain saturated hydrocarbon radicals having from 1 to 4 carbon atoms such as for example methyl, ethyl, 1-propyl, 2-propyl, 1-butyl, 2-butyl, 2-methyl-1-propyl; “C₁₋₆alkyl” encompasses C₁₋₄alkyl radicals and the higher homologues thereof having 5 or 6 carbon atoms such as, for example, 1-pentyl, 2-pentyl, 3-pentyl, 1-hexyl, 2-hexyl, 2-methyl-1-butyl, 2-methyl-1-pentyl, 2-ethyl-1-butyl, 3-methyl-2-pentyl, and the like. The term “C₁₋₁₀alkyl” as a group or part of a group encompasses C₁₋₆alkyl radicals and the higher homologues thereof having from 7 to 10 carbon atoms such as, for example, 1-heptyl, 2-heptyl, 2-methyl-1-hexyl, 2-ethyl-1-hexyl, 1-octyl, 2,octyl, 2-methyl-1-heptyl, 2-methyl-2-heptyl, 1-nonyl, 2-nonyl, 2-methyl-1-octyl, 2-methyl-2-octyl, 1-decyl, 2-decyl, 3-decyl, 2-methyl-1-decyl and the like.

The term “C₂₋₆alkenyl” as a group or part of a group defines straight and branched chained hydrocarbon radicals having saturated carbon-carbon bonds and at least one double bond, and having from 2 to 6 carbon atoms, such as, for example, propenyl, buten-1-yl, buten-2-yl, 2-buten-1-yl, 3-buten-1-yl, penten-1-yl, penten-2-yl, 2-penten-2-yl, hexen-1-yl, hexen-2-yl, hexen-3-yl, 2-methylbuten-1-yl, 1-methyl-2-penten-1-yl and the like. The term “C₂₋₁₀alkenyl” as a group or part of a group defines comprises C₂₋₆alkenyl groups and the higher homologues thereof having from 7 to 10 carbon atoms and at least one double bond such as, for example, hepten-1-yl, 2-hepten-1-yl, 3-hepten-1-yl, octen-1-yl, 2-octen-1-yl, 3-octen-1-yl, nonen-1-yl, 2-nonen-1-yl, 3-nonen-1-yl, 4-nonen-1-yl, decen-1-yl, 2-decen-1-yl, 3-decen-1-yl, 4-decen-1-yl, 1-methyl-2-hexen-1-yl and the like. Preferred are C₂₋₆alkenyl or C₂₋₁₀alkenyl groups having one double bond. Whenever liked to a heteroatom, the C₂₋₆alkenyl or C₂₋₁₀alkenyl groups by preference are linked to the hetero atom by a saturated carbon atom. Preferred subgroups amongst C₂₋₆alkenyl or C₂₋₁₀alkenyl are C₃₋₆alkenyl or C₃₋₁₀alkenyl which are alkenyl groups as specified herein having from 3 to 6 or from 3 to 10 carbon atoms.

The term “C₃₋₇cycloalkyl” is generic to cyclopropyl, cyclobutyl, cyclopentyl, cyclohexyl and cycloheptyl.

The term “C₁₋₄alkanediyl” defines bivalent straight and branched chain saturated hydrocarbon radicals having from 1 to 4 carbon atoms such as, for example, methylene, 1,2-ethanediyl, 1,3-propanediyl, 1,4-butanediyl, 1,2-propanediyl, 2,3-butanediyl, and the like, refers to bivalent C₁₋₄alkyl radicals having from one to four carbon atoms, in particular methylene, 1,2-ethanediyl, 1,1-ethanediyl, 1,2-propanediyl, 1,3-propanediyl, 1,2-butanediyl, 1,3-butanediyl, 1,4-butanediyl. “C₂₋₄alkanediyl” similarly refers to bivalent hydrocarbon atoms having 2 to 4 carbon atoms. Of particular interest are the C₁₋₄alkanediyl groups in which the carbon atoms bearing the connecting bond are next to one another (in vicinal position), these groups sometimes being referred to as ethylene, propylene and butylene.

“C₂₋₄alkanedioxy” refers to straight and branched chain saturated hydrocarbon radicals having 2-4 carbon atoms and two oxy (—O—) groups, e.g. 1,2-ethanedioxy (—O—CH₂—CH₂—O—), 1,3-propanedioxy (—O—CH₂CH₂CH₂—O—), 1,2-propanedioxy (—O—CH₂—CH(CH₃)—O—), 1,4-butanedioxy (—O—CH₂CH₂CH₂CH₂—O—), and the like.

The terms “spiro(C₂₋₄alkanedioxy)” and “spiro(diC₁₋₄alkyloxy)” refer to a linkage of the C₂₋₄alkanedioxy and diC₁₋₄alkyloxy groups to the same carbon atom, whereby in the former instance a ring is formed.

The term “halo” is generic to fluoro, chloro, bromo or iodo.

A hydroxyC₁₋₆alkyl group when substituted on an oxygen atom or a nitrogen atom preferably is a hydroxyC₂₋₆alkyl group wherein the hydroxy group and the oxygen or nitrogen are separated by at least two carbon atoms.

The term “methanimidamidyl” is used in accordance with the Chemical Abstracts Nomenclature (CAS) and refers to the radical of formula H₂N—C(═NH)—, which radical can also be referred to as “amidine”. Likewise N-hydroxy-methanimidamidyl is used in accordance with the CAS nomenclature and refers to the radical of formula H₂N—C(═N—OH)— or its tautomer HN═C(—NH—OH)—, which radical can also be referred to as “hydroxyamidine”.

The term “hydroxycarbonyl” refers to a carboxyl group (—COOH).

The aryl group is phenyl optionally substituted with one or more substituents and in particular is phenyl optionally substituted with one, two, three, four or five substituents, preferably phenyl substituted with one, two or three substituents.

Het₁ in particular is a 5-membered ring system as specified above wherein the ring system is aromatic. More particularly, Het₁ is a 5-membered ring system as specified above wherein the ring system contains one oxygen, sulfur or nitrogen, and optionally one, two or three further nitrogen atoms and wherein the remaining ring members are carbon atoms. Further in particular, Het₁ is an aromatic 5-membered ring system as specified above wherein the ring system contains one oxygen, sulfur or nitrogen atom, and optionally one, two or three further nitrogen atoms and wherein the remaining ring members are carbon atoms. In each of the instances mentioned in this paragraph, Het₁ may be optionally substituted with any of substituents specified herein in the definitions of the compounds of formula (I) as well as any of the subgroups of compounds of formula (I).

Examples of Het₁ rings are furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, each of which individually and independently may be optionally substituted with a substituent selected from the group consisting of C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl.

The substituents R¹², R¹³, —COOR⁴, R^{13a}, R¹⁸, R¹⁹ on radicals (a-2), (a-3), (a-5), (a-6) and (b-1) may be positioned at any ring carbon atom, including the atoms of radicals Q¹. Preferably, the substituents R¹², R¹³, R^{13a}, R¹⁸ or R¹⁹ are not in α-position from the ring nitrogen atom, in particular where any of said substituents is oxo, spiro(C₂₋₄alkanediyldioxy), spiro(diC₁₋₄alkyloxy), —NR^{5a}R^{5b}, hydroxy or C₁₋₄alkyloxy. Of particular interest are radicals (a-2), (a-3), (a-5), (a-6) and (b-1) wherein substituents R², R¹³, R^{13a}, R¹⁸ or R¹⁹ are positioned on a carbon atom of Q¹ or where Q¹ is a direct bond, on the ring carbon atom to which Q¹ is linked.

The connecting bond in radicals (a-3), (a-4) and (a-6) may be positioned at any ring carbon atom, including the atoms of radicals Q¹.

It should be noted that different isomers of the various heterocycles may exist within the definitions as used throughout the specification. For example, oxadiazolyl may be 1,2,4-oxadiazolyl or 1,3,4-oxadiazolyl or 1,2,3-oxadiazolyl; likewise for thiadiazolyl which may be 1,2,4-thiadiazolyl or 1,3,4-thiadiazolyl or 1,2,3-thiadiazolyl; pyrrolyl may be 1H-pyrrolyl or 2H-pyrrolyl.

It should also be noted that the radical positions on any molecular moiety used in the definitions may be anywhere on such moiety as long as it is chemically stable. For instance pyridyl includes 2-pyridyl, 3-pyridyl and 4-pyridyl; pentyl includes 1-pentyl, 2-pentyl and 3-pentyl, morpholinyl includes 4-morpholinyl, 3-morpholinyl and 2-morpholinyl.

When any variable (e.g. halogen or C₁₋₄alkyl) occurs more than one time in any constituent, each definition is independent.

The term “prodrug” as used throughout this text means the pharmacologically acceptable derivatives such as esters, amides and phosphates, such that the resulting in vivo biotransformation product of the derivative is the active drug as defined in the compounds of formula (I). The reference by Goodman and Gilman (The Pharmacological Basis of Therapeutics, 8^{th} ed, McGraw-Hill, Int. Ed. 1992, “Biotransformation of Drugs”, p 13-15) describing prodrugs generally is hereby incorporated. Prodrugs preferably have excellent aqueous solubility, increased bioavailability and are readily metabolized into the active inhibitors in vivo. Prodrugs of a compound of the present invention may be prepared by modifying functional groups present in the compound in such a way that the modifications are cleaved, either by routine manipulation or in vivo, to the parent compound.

Preferred are pharmaceutically acceptable ester prodrugs that are hydrolysable in vivo and are derived from those compounds of formula (I) having a hydroxy or a carboxyl groups. An in vivo hydrolysable ester is an ester, which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆ alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

For therapeutic use, the salts of the compounds of formula (I) are those wherein the counterion is pharmaceutically or physiologically acceptable. However, salts having a pharmaceutically unacceptable counterion may also find use, for example, in the preparation or purification of a pharmaceutically acceptable compound of formula (I). All salts, whether pharmaceutically acceptable or not are included within the ambit of the present invention.

The pharmaceutically acceptable or physiologically tolerable addition salt forms which the compounds of the present invention are able to form can conveniently be prepared using the appropriate acids, such as, for example, inorganic acids such as hydrohalic acids, e.g. hydrochloric or hydrobromic acid; sulfuric; hemisulphuric, nitric; phosphoric and the like acids; or organic acids such as, for example, acetic, aspartic, dodecyl-sulphuric, heptanoic, hexanoic, nicotinic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-amino-salicylic, pamoic and the like acids.

Conversely said acid addition salt forms can be converted by treatment with an appropriate base into the free base form.

The compounds of formula (I) containing an acidic proton may also be converted into their non-toxic metal or amine addition base salt form by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

Conversely said base addition salt forms can be converted by treatment with an appropriate acid into the free acid form.

The term “salts” also comprises the hydrates and the solvent addition forms that the compounds of the present invention are able to form. Examples of such forms are e.g. hydrates, alcoholates and the like.

The term “quaternary ammonium salt” as used herein defines the quaternary ammonium salts which the compounds of formula (I) are able to form by reaction between a basic nitrogen of a compound of formula (I) and an appropriate quaternizing agent, such as, for example, an optionally substituted alkyl halide, aryl halide or arylalkyl halide, e.g. methyliodide or benzyliodide. Other reactants with good leaving groups may also be used, such as alkyl trifluoromethane sulfonates, alkyl methane sulfonates, and alkyl p-toluenesulfonates. A quaternary amine has a positively charged nitrogen. Pharmaceutically acceptable counterions include chloro, bromo; iodo, trifluoroacetate and acetate. The counterion of choice can be introduced using ion exchange resins.

Particular quaternary ammonium salts are those derived from the groups —NR⁷R⁸, —NR⁹R¹⁰, —N(R^{5a}R^{5b}), pyrrolidin-1-yl, piperidin-1-yl, homopiperidin-1-yl, 4-(C₁₋₄alkyl)-piperazin-1-yl, morpholin-4-yl-, NR^{16a}R^{16b}; or NR^{17a}R^{17b}. These quaternized groups can be represented by the formulae
—(NR⁷R⁸R^{8a})⁺, —(NR⁹R¹⁰R^{8a})⁺, —(NR^{5a}R^{5b}R^{8a})⁺, —(NR^{16a}R^{16b}R^{8a})⁺; —(NR^{17a}R^{17b}R^{8a})⁺, 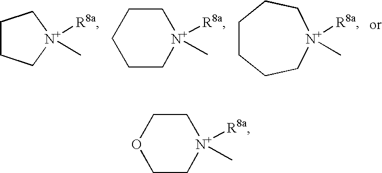
wherein each R^{8a} independently is C₁₋₆alkyl, arylC₁₋₆alkyl or hydroxyC₁₋₆alkyl, in particular each R^{8a} independently is C₁₋₆alkyl or arylC₁₋₆alkyl.

The N-oxide forms of the present compounds are meant to comprise the compounds of formula (I) wherein one or several nitrogen atoms are oxidized to the so-called N-oxide.

Some of the present compounds may also exist in tautomeric forms. Such forms, although not explicitly indicated in the above formula are intended to be included within the scope of the present invention. For example, within the definition of Het, a 5 membered aromatic heterocycle such as for example an 1,2,4-oxadiazole may be substituted with a hydroxy or a thio group in the 5-position, thus being in equilibrium with its respective tautomeric form as depicted below.

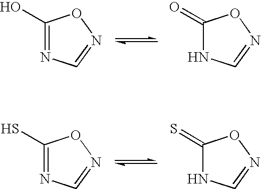

The term stereochemically isomeric forms of compounds of the present invention, as used hereinbefore, defines all possible compounds made up of the same atoms bonded by the same sequence of bonds but having different three-dimensional structures which are not interchangeable, which the compounds of the present invention may possess. Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of the present invention both in pure form or in admixture with each other are intended to be embraced within the scope of the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term ‘stereoisomerically pure’ concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stercoisomeric excess of 97% up to 100%. The terms ‘enantiomerically pure’ and ‘diastereomerically pure’ should be understood in a similar way, but then having regard to the enantiomeric excess, respectively the diastereomeric excess of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound may be synthesized by stereospecific methods of preparation. These methods will advantageously employ enantiomerically pure starting materials.

The diastereomeric racemates of formula (I) can be obtained separately by conventional methods. Appropriate physical separation methods that may advantageously be employed are, for example, selective crystallization and chromatography, e.g. column chromatography.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

Whenever used hereinafter, the term “compounds of formula (I)”, or “the present compounds” or similar term is meant to include the compounds of general formula (I), their N-oxides, salts, stereoisomeric forms, racemic mixtures, prodrugs, esters and metabolites, as well as their quaternized nitrogen analogues. One embodiment of the invention are the subgroups comprising the N-oxides of the compounds of formula (I) or of any subgroup of the compounds of formula (I) specified herein, including any salts or stereoisomeric forms thereof.

It is to be understood that any of the subgroups of compounds of formulae (I) is meant to also comprise any prodrugs, N-oxides, addition salts, quaternary amines and stereochemically isomeric forms of such compounds.

Embodiments of the present invention are those compounds of formula (I) or any of the subgroups of compounds of formula (I) specified herein, wherein
(1) n is 1 or 2; or wherein:
(1-a) n is 1.

Further embodiments of the present invention are those compounds of formula (I) or any of the subgroups of compounds of formula (I) specified herein, wherein
(2) R¹ is hydrogen, cyano, halo, aminocarbonyl, C₁₋₄alkylaminocarbonyl, hydroxycarbonyl, C₁₋₄alkyloxycarbonyl, arylaminocarbonyl, N-hydroxy-methanimidamidyl, mono- or di(C₁₋₄alkyl)methanimidamidyl, Het₁ or Het₂;
(2-a) R¹ is hydrogen, cyano, halo, aminocarbonyl, C₁₋₄alkylaminocarbonyl, arylaminocarbonyl, C₁₋₄alkyloxycarbonyl, N-hydroxy-methanimidamidyl, Het₁ or pyridinyl;
(2-b) R¹ is hydrogen, cyano, halo, aminocarbonyl, C₁₋₄alkylaminocarbonyl, arylaminocarbonyl, C₁₋₄alkyloxycarbonyl, N-hydroxy-methanimidamidyl, pyridinyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, each of which individually and independently may be optionally substituted with a substituent selected from the group consisting of C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl;
(2-c) R¹ is hydrogen, cyano, halo, aminocarbonyl, C₁₋₄alkylaminocarbonyl, arylaminocarbonyl, C₁₋₄alkyloxycarbonyl, N-hydroxy-methanimidamidyl, pyridinyl, furanyl, thienyl, oxadiazolyl, tetrazolyl, wherein the latter four may be optionally substituted with C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio;
(2-d) R¹ is hydrogen, cyano, bromo, tetrazolyl or oxadiazolyl optionally substituted with a substituent selected from the group consisting of C₁₋₄alkyl, C₂₋₆alkenyl, C₃₋₇cycloalkyl, hydroxy, C₁₋₄alkoxy, amino, cyano, trifluoromethyl, hydroxyl-C₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio;
(2-e) R¹ is hydrogen, cyano, halo, aminocarbonyl, C₁₋₄alkylaminocarbonyl, arylaminocarbonyl, C₁₋₄alkyloxycarbonyl, N-hydroxy-methanimidamidyl, pyridinyl, furanyl, tetrazolyl, oxadiazolyl, wherein the latter may be optionally substituted with C₁₋₄alkyl, halo, amino, cyano, trifluoromethyl, hydroxyC₁₋₄alkyl, cyanoC₁₋₄alkyl, aminoC₁₋₄alkyl, mono- or di(C₁₋₄alkyl)aminoC₁₋₄alkyl, arylC₁₋₄alkyl, aminoC₂₋₆alkenyl mono- or di(C₁₋₄alkyl)aminoC₂₋₆alkenyl, oxo, thio;
(2-f) R¹ is hydrogen, cyano, halo, aminocarbonyl, C₁₋₄alkylaminocarbonyl, arylaminocarbonyl, C₁₋₄alkyloxycarbonyl, N-hydroxy-methanimidamidyl, pyridinyl, furanyl, tetrazolyl, oxadiazolyl, wherein the latter may be optionally substituted with C₁₋₄alkyl, trifluoromethyl, aminoC₂₋₆alkenyl, mono- or di(C₁₋₄alkyl)aminoC₂₋₆alkenyl, oxo, thio;
(2-g) R¹ is cyano, aminocarbonyl, C₁₋₄alkylaminocarbonyl;
(2-h) R¹ is cyano, methyloxycarbonyl, methylaminocarbonyl, ethyloxycarbonyl or ethylaminocarbonyl; or
(2-i) R¹ is cyano and ethylaminocarbonyl; or
(2-j) R¹ is cyano.

Further embodiments of the present invention are those compounds of formula (I) or any of the subgroups of compounds of formula (I) specified herein, wherein:
(3) X is O or S; or
(3-a) X is O;

Further embodiments of the present invention are those compounds of formula (I) or any of the subgroups of compounds of formula (I) specified herein, wherein:
(4) X is NR², wherein R² is aryl substituted with a radical —COOR⁴; or
   R² is C₁₋₁₀alkyl, C₂₋₁₀alkenyl, C₃₋₇cycloalkyl, wherein said C₁₋₁₀alkyl, C₂₋₁₀alkenyl, C₃₋₇cycloalkyl, each individually and independently, is substituted with aryl wherein said aryl is substituted with a radical —COOR⁴; or
   wherein said C₁₋₁₀alkyl, C₂₋₁₀alkenyl, C₃₋₇cycloalkyl, each individually and independently, is substituted with a radical selected from NR^{5a}—C(═NR^{5b})—NR^{5c}R^{5d}, —NR^{5a}—C(═NR^{5e})—R^{5f}, —O—NR^{5a}—C(═NR^{5b})—NR^{5c}R^{5d}, —O—NR^{5a}—C(═NR^{5e})—R^{5f}, -sulfonyl-R⁶, —NR⁷R⁸, —NR⁹R¹⁰, a radical 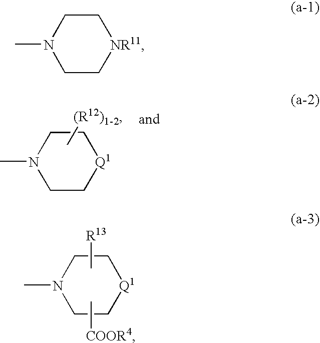
   wherein
   each Q¹ independently is a direct bond, —H₂—, or —CH₂₋₂—,
   each R⁴ independently is hydrogen, C₁₋₄alkyl, arylC₁₋₄alkyl;
   each R^{5a}, R^{5b}, R^{5c}, R^{5d} independently is hydrogen, C₁₋₄alkyl or arylC₁₋₄alkyl;
   each R^{5e}, R^{5f} independently is hydrogen, C₁₋₄alkyl or arylC₁₋₄alkyl, or R^{5e} and R^{5f}, taken together may form a bivalent alkylene radical of formula —CH₂—CH₂— or —CH₂—CH₂—CH₂—;
   R⁶ is C₁₋₄alkyl, —N(R^{5a}R^{5b}), C₁₋₄alkyloxy, pyrrolidin-1-yl, piperidin-1-yl, homopiperidin-1-yl, piperazin-1-yl, 4-C₁₋₄alkyl)-piperazin-1-yl, morpholin-4-yl-, thiomorpholinyl-4-yl-, 1-oxothiomorpholin-4-yl and 1,1-dioxo-thiomorpholinyl;
   R⁷ is hydrogen or hydroxyC₁₋₄alkyl;
   R⁸ is hydroxyC₁₋₄alkyl;
   R⁹ is hydrogen or C₁₋₄alkyl;
   R¹⁰ is Het₁, Het₂ or a radical 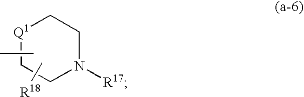
   R¹¹ is aryl, arylC₁₋₄alkyl, formyl, C₁₋₄alkylcarbonyl, arylcarbonyl, arylC₁₋₄alkylcarbonyl, C₁₋₄alkyloxycarbonyl, arylC₁₋₄alkyloxycarbonyl, R^{5a}R^{5b}N-carbonyl, hydroxyC₁₋₄alkyl, C₁₋₄alkyloxyC₁₋₄alkyl, arylC₁₋₄alkyloxyC₁₋₄alkyl, aryloxyC₁₋₄alkyl, Het₂;
   R¹² is hydroxy, C₁₋₄alkyl, arylC₁₋₄alkyl, C₁₋₄alkyloxy, arylC₁₋₄alkyloxy, oxo, spiro(C₂₋₄alkylenedioxy), spiro(diC₁₋₄alkyloxy), —NR^{5a}R^{5b};
   R¹³ is hydrogen, hydroxy, C₁₋₄alkyl, C₁₋₄alkyloxy, or arylC₁₋₄alkyloxy; or
   R² is a radical of formula: 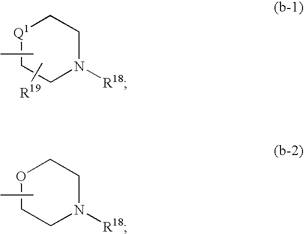 —CₚH₂ₚ—CH(OR¹⁴)—C_{q}H_{2q}—R¹⁵ (b-3);
      —CH₂—CH₂—(O—CH₂—CH₂)ₘ—OR¹⁴ (b-4);
      —CH₂—CH₂—(O—CH₂—CH₂)ₘ—NR^{5a}R^{5b} (b-5);
   wherein in radical (b-3) one of the hydrogen atoms in —CₚH₂ₚ— and one of the hydrogen atoms in CH(OR¹⁴)—C_{q}H_{2q}—, that is not part of R¹⁴, may be replaced by a direct bond or a C₁₋₄alkanediyl group;
   p is 1, 2 or 3;
   q is 0, 1, 2 or 3;
   m is 1 to 10;
   each R¹⁴ independently is hydrogen, C₁₋₄alkyl, aryl C₁₋₄alkyl, aryl, C₁₋₄alkylcarbonyl, —SO₃H, —PO₃H₂;
   R¹⁵ is a substituent selected from the group consisting of cyano, NR^{16a}R^{16b}, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl, pyridazinyl, triazinyl, hydroxy-carbonyl, C₁₋₄alkylcarbonyl, N(R^{16a}R^{16b})carbonyl, C₁₋₄alkyloxycarbonyl, pyrrolidin-1-ylcarbonyl, piperidin-1-ylcarbonyl, homopiperidin-1-ylcarbonyl, piperazin-1-ylcarbonyl, 4-(C₁₋₄alkyl)-piperazin-1-ylcarbonyl, morpholin-1-yl-carbonyl, thiomorpholin-1-yl-carbonyl, 1-oxothiomorpholin-1-ylcarbonyl and 1,1-dioxo-thiomorpholin-1-ylcarbonyl; and wherein R¹⁵ may additionally be aryl substituted with a radical —COOR⁴; or a radical selected from —NR^{5a}—C(═NR^{5b})—NR^{5c}R^{5d}, NR^{5a}—(═NR^{5e})—R^{5f}, —O—NR^{5a}—C(═NR^{5b})—NR^{5c}R^{5d}, —O—NR^{5a}—C(═NR^{5e}—R^{5f}, -sulfonyl-R⁶, —NR⁷R⁸, —NR⁹R¹⁰, a radical (a-1), (a-2), (a-3); wherein R⁴, R^{5a}, e R^{5b}, R^{5c}, R^{5d}, R⁶, R⁷, R⁸, R⁹, R¹⁰, and the radicals (a-1), (a-2), (a-3) independently are as defined above;
   R^{16a} is hydrogen, C₁₋₄alkyl or C₁₋₄alkyl substituted with a substituent selected from the group consisting of amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl, 1-oxothiomorpholinyl and 1,1-dioxo-thiomorpholinyl;
   R^{16b} is hydrogen, C₁₋₄alkyl or C₁₋₄alkyl substituted with a substituent selected from the group consisting of amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl, 1-oxothiomorpholinyl and 1,1-dioxo-thiomorpholinyl;
   each R¹⁸ independently is hydrogen, C₁₋₄alkyl or arylC₁₋₄alkyl;
   R¹⁹ is hydrogen, hydroxy, C₁₋₄alkyl or a radical —COOR⁴; or wherein
(4-a) X is NR² wherein R² is C₁₋₁₀alkyl, C₂₋₁₀alkenyl, C₃₋₇cycloalkyl, each of the former three radicals being independently substituted with aryl, wherein said aryl is substituted with a radical —COOR⁴; or
(4-a-1) X is NR² wherein R² is C₁₋₁₀alkyl being substituted with aryl, wherein said aryl is substituted with a radical —COOR⁴; or
(4-a-2) X is NR² wherein R¹ is C₁₋₆alkyl being substituted with phenyl substituted with a radical —COOR⁴; or
(4-a-3) X is NR² wherein R² is C₁₋₆alkyl being substituted with phenyl substituted in para position with a radical —COOR¹; or wherein
(4-b) X is NR² wherein R² is C₁₋₁₀alkyl, C₂₋₁₀alkenyl, C₃₋₇cycloalkyl, each of the former three radicals being independently substituted with a radical selected from —NR^{5a}—C(═NR^{5b})—NR^{5c}R^{5d}, —O—NR^{5a}—C(═NR^{5b})—NR^{5c}R^{5d}, -sulfonyl-R⁶, —NR⁷R⁸, —NR⁹R¹⁰, a radical (a-1), (a-2), (a-3), (a-4) and (a-5);
(4-b-1) X is NR² wherein R² is C₁₋₁₀alkyl substituted with a radical selected from —NR^{5a}—C(═NR^{5b})—NR^{5c}R^{5d}, —O—NR^{5a}—C(═NR^{5b})—NR^{5c}R^{5d}, -sulfonyl-R⁶, —NR⁷R⁸, —NR⁹R¹⁰, a radical (a-1), (a-2), (a-3), (a-4) and (a-5);
(4-b-2) X is NR² wherein R² is C₁₋₁₀alkyl substituted with a radical selected from —NR^{5a}—C(═NR^{5b})—NR^{5c}R^{5d}, —O—NR^{5a}—C(═NR^{5b})—NR^{5c}R^{5d}, -sulfonyl-R⁶, —NR⁷R⁸, —NR⁹R¹⁰, a radical (a-1), (a-2) and (a-3);
(4-b-3) X is NR² wherein R² is C₁₋₆alkyl substituted with the radicals mentioned in (4-b-1) or in (4-b-2)
(4-b-4) X is NR², wherein R² is C₁₋₆alkyl substituted with a radical selected from —NR^{5a}—(═NR^{5b})—NR^{5c}R^{5d}, —NR⁷R⁸, —NR⁹R¹⁰, a radical (a-1), (a-2), (a-3), (a-4) and (a-5);
(4-b-5) X is NR², wherein R² is C₁₋₆alkyl substituted with a radical selected from —NR^{5a}—C(═NR^{5b})—NR^{5c}R^{5d}, —NR⁷R⁸, —NR⁹R¹⁰, a radical (a-1), (a-2), (a-3);
(4-c) X is NR² wherein R² is a radical (b-1);
(4-c-1) X is NR² wherein R² is a radical (b-1), wherein R¹⁹ is hydrogen or —COOR⁴ and wherein Q¹ in radical (b-1) is a direct bond or CH₂—;
(4-d) X is NR² wherein R² is a radical (b-2);
(4-d-1) X is NR² wherein R² is a radical (b-2), wherein Q² is O;
(4-e) X is NR² wherein R² is a radical (b-3) wherein q is 1, 2 or 3;
(4-e-1) X is NR² wherein R² is a radical (b-3) wherein p is 1 and q is 1;
(4-e-2) X is NR² wherein R² is a radical (b-3) wherein R¹⁵ is cyano, NR^{16a}R^{16b}, pyrrolidinyl, piperidinyl, 4-(C₁₋₄alkyl)-piperazinyl, morpholinyl, aryl, imidazolyl, pyridyl, hydroxycarbonyl, N(R^{16a}R^{16b})carbonyl, C₁₋₄alkyloxycarbonyl 4-(C₁₋₄alkyl)-piperazin-1-ylcarbonyl, —NR⁷R⁸, —NR⁹R¹⁰, a radical (a-1), (a-2), (a-3), (a-4) or (a-5);
(4-e-3) X is NR² wherein R² is a radical (b-3) wherein R¹⁴ is hydrogen and R¹⁵ is cyano, NR^{16a}R^{16b}, pyrrolidinyl, piperidinyl, 4-(C₁₋₄alkyl)-piperazinyl, morpholinyl, aryl, imidazolyl, pyridyl, hydroxycarbonyl, N(R^{16a}R^{16b})carbonyl, C₁₋₄alkyloxycarbonyl or 4-(C₁₋₄alkyl)-piperazin-1-ylcarbonyl;
(4-e-4) X is NR² wherein R² is a radical (b-3) wherein p is 1 and q is 1, and R¹⁵ is cyano, NR^{16a}R^{16b}, pyrrolidinyl, piperidinyl, 4-morpholinyl, aryl, imidazolyl, pyridyl, hydroxycarbonyl or N(R^{16a}R^{16b})carbonyl;
(4-e-5) X is NR² wherein R² is a radical (b-3) R¹⁵ is NR^{16a}R^{16b}, pyrrolidinyl, piperidinyl, 4-morpholinyl;
(4-e-6) X is NR² wherein R² is a radical (b-3) wherein R¹⁵ is pyrrolidinyl, piperidinyl, 4-morpholinyl;
(4-e-6) X is NR² wherein R² is a radical (b-3) wherein R¹⁵ is pyrrolidinyl;
(4-f) X is NR² wherein R² is a radical (b-4) wherein m is 1-6;
(4-f-1) X is NR² wherein R² is a radical (b-4) wherein R¹⁴ is hydrogen or C₁₋₄alkyl;
(4-f-2) X is NIC wherein R² is a radical (b-4) wherein m is 1-5 and R¹⁴ is hydrogen or C₁₋₄alkyl;
(4-g) X is NR² wherein R² is a radical (b-5);
(4-g-1) X is NR² wherein R² is a radical (b-5) wherein m is 1-5.

Further embodiments of the present invention are those compounds of formula (I) or any of the subgroups of compounds of formula (I) specified herein, wherein
(5) R³ is hydrogen, nitro, cyano, amino, halo, hydroxy, C₁₋₄alkyloxy, C₁₋₄alkyl, hydroxycarbonyl, aminocarbonyl, mono- or di(C₁₋₄alkyl)aminocarbonyl, aminothiocarbonyl, C₁₋₄alkyloxycarbonyl, C₁₋₄alkylcarbonyl, mono- or di(C₁₋₄alkyl)methanimidamidyl, N-hydroxy-methanimidamidyl or Het₁;
(5-a) R³ is nitro, cyano, amino, halo, hydroxy, C₁₋₄alkyloxy, C₁₋₄alkyl, hydroxy-carbonyl, aminocarbonyl, di(C₁₋₄alkyl)aminocarbonyl, C₁₋₄alkyloxycarbonyl, mono- or di(C₁₋₄alkyl)methanimidamidyl, N-hydroxy-methanimidamidyl or Het₁;
(5-b) R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl or Het₁;
(5-c) R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl wherein each of said furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl may optionally be substituted with one or two substituents selected from the group consisting of C₁₋₄alkyl, C₂₋₆alkenyl, C₃₋₇cycloalkyl, hydroxy, C₁₋₄alkoxy, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl.
(5-d) R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl, oxadiazolyl, thienyl, thiazolyl, furanyl, isoxazolyl wherein each of said oxadiazolyl, thienyl, thiazolyl, furanyl, isoxazolyl may be substituted with a substituent selected from the group consisting of C₁₋₄alkyl, C₂₋₆alkenyl, C₃₋₇cycloalkyl, hydroxy, C₁₋₄alkoxy, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄-alkyl)aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl.
(5-e) R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl, oxadiazolyl, isoxazolyl, thienyl, pyrrolyl, triazolyl, thiazolyl, furanyl, isoxazolyl, tetrazolyl, wherein each of said oxadiazolyl, isoxazolyl, thienyl, pyrrolyl, triazolyl, thiazolyl, furanyl, isoxazolyl, tetrazolyl may optionally be substituted with C₁₋₄alkyl, C₂₋₆alkenyl, C₃₋₇cycloalkyl, hydroxy, C₁₋₄alkoxy, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl furanyl, isoxazolyl, C₁₋₄alkyl substituted isoxazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio;
(5-f) R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl, oxadiazolyl, isoxazolyl, thienyl, pyrrolyl, triazolyl, thiazolyl, furanyl, isoxazolyl, tetrazolyl, wherein each of said oxadiazolyl, isoxazolyl, thienyl, pyrrolyl, triazolyl, thiazolyl, furanyl, isoxazolyl, tetrazolyl may optionally be substituted with C₁₋₄alkyl, C₃₋₇cycloalkyl hydroxy, cyano, trifluoromethyl, cyanoC₁₋₄alkyl, mono- or di(C₁₋₄alkyl)amino, arylC₁₋₄alkyl di(C₁₋₄alkyl)aminoC₂₋₆alkenyl, furanyl, isoxazolyl, C₁₋₄alkyl substituted isoxazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio;
(5-g) R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl, oxadiazolyl, isoxazolyl, thiazolyl, furanyl, isoxazolyl, tetrazolyl, wherein each of said oxadiazolyl, isoxazolyl, thiazolyl, furanyl, isoxazolyl, tetrazolyl may optionally be substituted with C₁₋₄alkyl, hydroxy, cyano, trifluoromethyl;
(5-h) R³ is nitro, cyano, halo, C₁₋₄alkyloxy, hydroxycarbonyl, aminocarbonyl
(5-i) R³ is nitro;
(5-j) the R³ group on the phenyl ring is in the position vis-à-vis the nitrogen atom in the fused pyridine moiety.

Further embodiments of the present invention are those compounds of formula (I) or any of the subgroups of compounds of formula (II) specified herein, wherein:
(6) R⁴ is hydrogen or C₁₋₄alkyl; or wherein
(6-a) R⁴ is hydrogen.

Further embodiments of the present invention are those compounds of formula (I) or any of the subgroups of compounds of formula (I) specified herein, wherein:
(7) each R^{5a}, R^{5b}, R^{5c}, R^{5d}, R^{5e} and R^{5f} independently is hydrogen or C₁₋₄alkyl; or R^{5e} and R^{5f}, taken together may form a bivalent alkanediyl radical of formula —CH₂—CH₂— or —CH₂—CH₂—CH₂—;
(7-a) each R^{5a}, R^{5b}, R^{5c}, R^{5d}, R^{5e} and R^{5f} independently is hydrogen or C₁₋₄alkyl;
(7-b) each R^{5a}, R^{5b}, R^{5c}, R^{5d}, R^{5e} and R^{5f} independently is hydrogen.

Further embodiments of the present invention are those compounds of formula (I) or any of the subgroups of compounds of formula (I) specified herein, wherein:
(8) R⁶ is C₁₋₄alkyl, —N(R^{5a}R^{5b}), C₁₋₄alkyloxy, pyrrolidin-1-yl, piperidin-1-yl, homopiperidin-1-yl, piperazin-1-yl, 4-(C₁₋₄alkyl)-piperazin-1-yl, morpholin-4-yl-;
(8-a) R⁶ is C₁₋₄alkyl, —N(R^{5a}R^{5b}), C₁₋₄alkyloxy, pyrrolidin-1-yl, piperidin-1-yl, morpholin-4-yl-;
(8-b) R⁶ is C₁₋₄alkyl, —N(R^{5a}R^{5b}), pyrrolidin-1-yl, piperidin-1-yl, morpholin-4-yl-; wherein R^{5a} and R^{5b} independently are hydrogen or C₁₋₄alkyl.

Other embodiments of the present invention are those compounds of formula (I) or any of the subgroups of compounds of formula (I) specified herein, wherein one or more of the following restrictions apply:
(9-a) R⁷ is hydrogen or hydroxyC₁₋₄alkyl;
(9-b) R⁸ is hydroxyC₁₋₄alkyl;
(9-c) R⁹ is hydrogen.

Still other embodiments of the present invention are those compounds of formula (I) or any of the subgroups of compounds of formula (I) specified herein, wherein:
(10) R¹⁰ is Het₁, pyridyl, pyrimidinyl or a radical (a-6);
(10-a) R¹⁰ is imidazolyl, isoxazolyl, pyrazolyl, triazolyl, each of which may be optionally substituted with C₁₋₄alkyl; or R¹⁰ is pyrimidyl or pyrimidinyl or a radical (a-6);
(10-b) R¹⁰ is pyrimidyl, pyrimidinyl or a radical (a-6);
(10-c) R¹⁰ is a radical (a-6).

Still other embodiments of the present invention are those compounds of formula (I) or any of the subgroups of compounds of formula (I) specified herein, wherein:
(11) R¹¹ is aryl, arylC₁₋₄alkyl, formyl, C₁₋₄alkylcarbonyl, arylcarbonyl, C₁₋₄alkyloxycarbonyl, arylC₁₋₄alkyloxycarbonyl, mono- and diC₁₋₄alkylaminocarbonyl, C₁₋₄alkyloxyC₁₋₄alkyl, arylC₁₋₄alkyloxyC₁₋₄alkyl, pyridyl or pyrimidinyl;
(11-a) R¹¹ is aryl, arylC₁₋₄alkyl, formyl, C₁₋₄alkylcarbonyl, arylcarbonyl, C₁₋₄alkyloxycarbonyl, C₁₋₄alkyloxyC₁₋₄alkyl, arylC₁₋₄alkyloxyC₁₋₄alkyl, pyridyl or pyrimidinyl.
(11-b) R¹¹ is aryl, C₁₋₄alkylcarbonyl, C₁₋₄alkyloxycarbonyl, hydroxyC₁₋₄alkyl or pyridyl.

Still other embodiments of the present invention are those compounds of formula (I) or any of the subgroups of compounds of formula (I) specified herein, wherein:
(12) R¹² is hydroxy, C₁₋₄alkyl, C₁₋₄alkyloxy, oxo, spiro(C₂₋₄alkanediyldioxy), spiro(diC₁₋₄alkyloxy), —NR^{5a}R^{5b};
(12-a) when in radical (a-2) one R¹² radical is present, R¹² is hydroxy, C₁₋₄alkyl, C₁₋₄alkyloxy, oxo, —NR^{5a}R^{5b}; or when in radical (a-2) two R¹² radicals are present both independently are C₁₋₄alkyl, spiro(C₂₋₄alkanediyldioxy) or spiro(diC₁₋₄alkyloxy); and
(12-b) R¹² is hydroxy or C₁₋₄alkyl.

Still other embodiments of the present invention are those compounds of formula (I) or any of the subgroups of compounds of formula (I) specified herein, wherein one or more of the following restrictions apply:
(13-a) Q¹ is a direct bond or —CH₂—; or
(13-b) Q² is O or S; or (13-b-1) Q² is O.

Still other embodiments of the present invention are those compounds of formula (I) or any of the subgroups of compounds of formula (I) specified herein, wherein one or more of the following restrictions apply:
(14-a) R¹³ is hydrogen or hydroxy;
(14-b) R^{13a} is C₁₋₄alkyl;
(14-c) R^{13b} is hydrogen;

Still other embodiments of the present invention are those compounds of formula (I) or any of the subgroups of compounds of formula (I) specified herein, wherein:
(15) R¹⁴ is hydrogen, C₁₋₄alkyl or arylC₁₋₄alkyl;
(15-a) R¹⁴ is hydrogen or C₁₋₄alkyl;
(15-b) R¹⁴ is hydrogen.

Still further embodiments of the present invention are those compounds of formula (I) or any of the subgroups of compounds of formula (I) specified herein, wherein:
(16) R¹⁵ is selected from the group consisting of cyano, NR^{16a}R^{16b}, pyrrolidinyl, piperidinyl, homopiperidinyl, piperazinyl, 4-(C₁₋₄alkyl)-piperazinyl, 4-(C₁₋₄alkylcarbonyl)-piperazinyl, 4-(C₁₋₄alkyloxycarbonyl)-piperazinyl, morpholinyl, thiomorpholinyl, 1-oxothiomorpholinyl, 1,1-dioxo-thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl, pyridazinyl, triazinyl, hydroxy-carbonyl, C₁₋₄alkylcarbonyl, N(R^{16a}R^{16b})carbonyl, C₁₋₄alkyloxycarbonyl, pyrrolidin-1-yl-carbonyl, piperidin-1-ylcarbonyl, homopiperidin-1-ylcarbonyl, piperazin-1-yl-carbonyl, 4-(C₁₋₄alkyl)-piperazin-1-ylcarbonyl, morpholin-1-yl-carbonyl, thiomorpholin-1-yl-carbonyl, 1-oxothiomorpholin-1-ylcarbonyl and 1,1-dioxo-thiomorpholin-1-ylcarbonyl; or R¹⁵ may additionally be aryl substituted with a radical —COOR⁴; or a radical selected from NR^{5a}—C(═NR^{5b})—NR^{5c}R^{5d}, —NR^{5a}—C(═NR^{5e})—NR^{5f}, —O—NR^{5a}—C(═NR^{5b})—NR^{5c}R^{5d}, —O—NR^{5a}—C(═NR^{5e})—R^{5f}, -sulfonyl-R⁶, —NR⁷R⁸, —NR⁹R¹⁰, a radical (a-1), (a-2), (a-3);
(16-a) R¹⁵ is selected from the group consisting of cyano, NR^{16a}R^{16b}, pyrrolidinyl, piperidinyl, homopiperidinyl, piperazinyl, 4-(C₁₋₄alkyl)-piperazinyl, 4-(C₁₋₄alkylcarbonyl)-piperazinyl, 4-(C₁₋₄alkyloxycarbonyl)-piperazinyl, morpholinyl, thiomorpholinyl, 1-oxothiomorpholinyl, 1,1-dioxo-thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl pyridazinyl, triazinyl, hydroxy-carbonyl, C₁₋₄alkylcarbonyl, N(R^{16a}R^{16b})carbonyl, C₁₋₄alkyloxycarbonyl, pyrrolidin-1-yl-carbonyl, piperidin-1-ylcarbonyl, homopiperidin-1-ylcarbonyl, piperazin-1-yl-carbonyl, 4-(C₁₋₄alkyl)-piperazin-1-ylcarbonyl, morpholin-1-yl-carbonyl, thiomorpholin-1-yl-carbonyl, 1-oxothiomorpholin-1-ylcarbonyl and 1,1-dioxo-thiomorpholin-1-ylcarbonyl;
(16-b) R¹⁵ is selected from the group consisting of cyano, NR^{16a}R^{16b}, pyrrolidinyl, piperidinyl, homopiperidinyl, piperazinyl, 4-(C₁₋₄alkyl)-piperazinyl, 4-(C₁₋₄alkylcarbonyl)-piperazinyl, 4-(C₁₋₄alkyloxycarbonyl)-piperazinyl, morpholinyl, thiomorpholinyl, 1-oxothiomorpholinyl, 1,1-dioxo-thiomorpholinyl, aryl, hydroxy-carbonyl, C₁₋₄alkylcarbonyl, N(R^{16a}R^{16b})carbonyl, C₁₋₄alkyloxycarbonyl;
(16-c) R¹⁵ is selected from the group consisting of NR^{16a}R^{16b}, pyrrolidinyl, piperidinyl, homopiperidinyl, piperazinyl, 4-(C₁₋₄alkyl)-piperazinyl, 4-(C₁₋₄alkylcarbonyl)-piperazinyl, morpholinyl, thiomorpholinyl, 1-oxothiomorpholinyl, 1,1-dioxo-thiomorpholinyl;
(16-d) R¹⁵ is selected from the group consisting of NR^{16a}R^{16b}, pyrrolidinyl, piperidinyl, piperazinyl, 4-(C₁₋₄alkyl)-piperazinyl, morpholinyl, thiomorpholinyl, 1,1-dioxo-thiomorpholinyl;
(16-e) R¹⁵ is selected from the group consisting of pyrrolidinyl, piperidinyl.

Further embodiments of the present invention are those compounds of formula (I) or any of the subgroups of compounds of formula (I) specified herein, wherein:
(17) R^{16a} and R^{16b} independently from one another are hydrogen, C₁₋₄alkyl or C₁₋₄alkyl substituted with a substituent selected from the group consisting of amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl and aryl;
(17-a) R^{16a} and R^{16b} independently from one another are hydrogen, C₁₋₄alkyl or C₁₋₄alkyl substituted with a substituent selected from the group consisting of amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl;
(17-b) R^{16a} and R^{16b} independently from one another are hydrogen or C₁₋₄alkyl.

Further embodiments of the present invention are those compounds of formula (I) or any of the subgroups of compounds of formula (I) specified herein, wherein:
(18) R^{17a} and R^{17b} independently from one another are hydrogen, C₁₋₄alkyl or arylC₁₋₄alkyl; or R^{17a} and R^{17b} together with the nitrogen atom to which they are attached form a pyrrolidinyl, piperidinyl, homopiperidinyl, morpholinyl, thiomorpholinyl, 1,1-dioxo-thiomorpholinyl, piperazinyl or 4-C₁₋₄alkyl-piperazinyl ring;
(18-a) R^{17a} and R^{17b} independently from one another are hydrogen, C₁₋₄alkyl or arylC₁₋₄alkyl;
(18-b) R^{17a} and R^{17b} independently from one another are hydrogen, C₁₋₄alkyl or arylC₁₋₄alkyl.

Further embodiments of the present invention are those compounds of formula (I) or any of the subgroups of compounds of formula (I) specified herein, wherein:
(19) each R¹⁸ independently is hydrogen, C₁₋₄alkyl or arylC₁₋₄alkyl;
(19-a) each R¹⁸ independently is hydrogen.

Further embodiments of the present invention are those compounds of formula (I) or any of the subgroups of compounds of formula (I) specified herein, wherein:
(20) R¹⁹ is hydrogen, C₁₋₄alkyl or a radical —COOR⁴;
(20-a) R¹⁹ is hydrogen.

Further embodiments of the present invention are those compounds of formula (I) or any of the subgroups of compounds of formula (I) specified herein, wherein:
(21) aryl is phenyl optionally substituted with one or more substituents each individually selected from the group consisting of C₁₋₆alkyl, C₁₋₄alkoxy, cyano, nitro;
(21-a) aryl is phenyl optionally substituted with one, two or three substituents each independently selected from C₁₋₆alkyl, C₁₋₄alkoxy, cyano and nitro;

Further embodiments of the present invention are those compounds of formula (I) or any of the subgroups of compounds of formula (I) specified herein, wherein:
(22) Het₁ is an aromatic 5-membered ring system wherein one, two, three or four ring members are heteroatoms each individually and independently selected from the group consisting of nitrogen, oxygen and sulfur, and wherein the remaining ring members are carbon atoms; and, where possible, any nitrogen ring member may optionally be substituted with C₁₋₄alkyl; any ring carbon atom may, each individually and independently, optionally be substituted with a substituent selected from the group consisting of C₁₋₄alkyl, C₃₋₇cycloalkyl, halo, cyano, trifluoromethyl, cyanoC₁₋₄alkyl, mono- or di(C₁₋₄alkyl)amino, mono- or di(C₁₋₄alkyl)aminoC₂₋₆alkenyl, isoxazolyl, aryl, hydroxycarbonyl, C₁₋₄alkyloxycarbonyl, oxo, thio; and wherein the foregoing isoxazolyl may optionally be substituted with C₁₋₄alkyl;
(22-a) Het₁ is furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, each of which individually and independently may be optionally substituted with a substituent selected from the group consisting of C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl.

Further embodiments of the present invention are those compounds of formula (I) or any of the subgroups of compounds of formula (I) specified herein, wherein:
(23) Het₂ is pyridyl or pyrimidinyl both optionally substituted with C₁₋₄alkyl.
(23-a) Het₂ is pyridyl or pyrimidinyl;
(23-b) Het₂ is pyridyl.

Further embodiments of the present invention are those compounds of formula (I) or any of the subgroups of compounds of formula (I) specified herein, wherein:
(24) p is 1, 2;
(24-a) p is 1.

Further embodiments of the present invention are those compounds of formula (I) or any of the subgroups of compounds of formula (I) specified herein, wherein:
(25) p is 1, 2, 3;
(25-a) q is 1, 2;
(25-b) q is 1.

Further embodiments of the present invention are those compounds of formula (I) or any of the subgroups of compounds of formula (I) specified herein, wherein:
(26) m is 1-8;
(26-a) m is 1-6;
(26-b) m is 1-4;
(26-c) m is 1-3;
(26-d) m is 1-2.

It is to be understood that subgroups of compounds of formula (I) comprise those groups of compounds of formula (I) wherein one or more of the above restrictions apply in whatever combination. If within a definition of a restriction one or more variables are present, each of these variables can have any of the meanings given in the restrictions relating to these variables. For example if within the restrictions for R² a radical NR^{5a}R^{5b} is mentioned the radicals R^{5a} and R^{5b} can have any of the meanings listed in the restrictions relating to R^{5a} and R^{5b}.

A particular group of compounds of formula (I) is this wherein R¹, R³ and n are as specified in the definition of the compounds of formula (I) and R² is as in restriction (4).

In one embodiment, n is 1 and the R³ group on the phenyl ring in the compounds of formula (I) or any subgroup specified herein, is in para-position vis-à-vis the nitrogen atom in the fused pyridine moiety as depicted herein below and hereinafter referred to as compounds of formula (I-a)

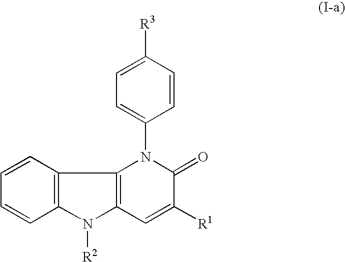

Another subgroup of the compounds of formula (I-a) are those compounds of formula (I-a), hereinafter referred to compounds of formula (I-a-1), wherein R³ is nitro.

Examples of subgroups of compounds are the following:

(i) those compounds of formula (I-a) wherein R³ is nitro and R¹ is cyano, halo, aminocarbonyl, N-hydroxy-methanimidamidyl, Het₁; further subgroups among the latter compounds are those compounds of formula (I-a) wherein R³ is nitro, X is O, or X is NR² wherein R² is a radical (b-3) wherein R¹⁴ is hydrogen and R¹⁵ is cyano, NR^{16a}R^{16b}, pyrrolidinyl, piperidinyl, 4-(C₁₋₄alkyl)-piperazinyl, morpholinyl, hydroxycarbonyl; or X is NR² wherein R² is a radical (b-4) wherein R¹⁴ is hydrogen or C₁₋₄alkyl; and R¹ is as in restrictions (2-d) to (2-j);

(ii) those compounds of formula (I-a) wherein R³ is nitro and R¹ is cyano and X is O. Suitable compounds are those compounds of formula (I-a) wherein R¹ is cyano and R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl or Het₁;

(iii) those compounds of formula (I-a) wherein R¹ is cyano; X is O; or X is NR² wherein R² is a radical of formula (b-3) wherein p is 1, q is 1, R¹⁴ is hydrogen, R¹⁵ is cyano, NR^{16a}R^{16b}, pyrrolidinyl, piperidinyl, 4-(C₁₋₄alkyl)-piperazinyl, morpholinyl, aryl, imidazolyl, pyridyl, hydroxycarbonyl, N(NR^{16a}R^{16b})carbonyl, C₁₋₄alkyloxycarbonyl or 4-(C₁₋₄alkyl)-piperazin-1-ylcarbonyl; or X is NR² wherein R² is a radical of formula (b-4) wherein m is 1, 2 or 3, R¹⁴ is hydrogen or C₁₋₄alkyl; and R³ is as in restrictions (5-d), (5-e), (5-f) or (5-g).

Another subgroup of compounds comprises those compounds of formula (I) as a salt, wherein the salt is selected from trifluoroacetate, fumarate, chloroacetate, methanesulfonate, oxalate, acetate and citrate.

Preferred compounds are any of the compounds listed in tables 1 and 2, more in particular the compound numbers 1-9 and 43.

Compounds of particular interest are:
1-(4-Nitro-phenyl)-2-oxo-1,2-dihydro-benzo[4,5]furo[3,2-b]pyridine-3-carbonitrile,
5-(2-Hydroxy-3-piperidin-1-yl-propyl)-1-(4-nitro-phenyl)-2-oxo-2,5-dihydro-1H-pyrido[3,2-b]indole-3-carbonitrile,
5-(3-Diethylamino-2-hydroxy-propyl)-1-(4-nitro-phenyl)-2-oxo-2,5-dihydro-1H-pyrido[3,2-b]indole-3-carbonitrile,
5-[2-(2-Methoxy-ethoxy)-ethyl]-1-(4-nitro-phenyl)-2-oxo-2,5-dihydro-1H-pyrido-[3,2-b]indole-3-carbonitrile, and especially
5-(2-Hydroxy-3-pyrrolidin-1-yl-propyl)-1-(4-nitro-phenyl)-2-oxo-2,5-dihydro-1H-pyrido[3,2-b]indole-3-carbonitrile, and especially
5-(2-Hydroxy-3-morpholin-4-yl-propyl)-1-(4-nitro-phenyl)-2-oxo-2,5-dihydro-1H-pyrido[3,2-b]indole-3-carbonitrile.

Other compounds of interest comprise the above compounds of interest and the salts and possible stereoisomers thereof; or the above compounds of interest and the N-oxides, salts and possible stereoisomers thereof.

The compounds of the present invention inhibit HIV reverse transcriptase and may also inhibit reverse transcriptases having similarity to HIV reverse transcriptase. Such similarity may be determined using programs known in the art including BLAST. In one embodiment, the similarity at the amino acid level is at least 25%, interestingly at least 50%, more interestingly at least 75%. In another embodiment, the similarity at the amino acid level at the binding pocket, for the compounds of the present invention, is at least 75%, in particular at least 90% as compared to HIV reverse transcriptase. Compounds of the present invention may be tested in other lentiviruses besides HIV-1, such as, for example, SIV and HIV-2.

The compounds of the present invention may have a good selectivity as measured by the ratio between EC₅₀ and CC₅₀ as described and exemplified in the antiviral analysis example. The compounds of the present invention have also a favorable specificity. There exists a high dissociation between the activity on lentiviruses versus other retroviridae, such as MLV, and versus non-viral pathogens.

The standard of “sensitivity” or alternatively “resistance” of a HIV reverse transcriptase enzyme to a drug is set by the commercially available HIV reverse transcriptase inhibitors. Existing commercial HIV reverse transcriptase inhibitors including efavirenz, nevirapine and delavirdine may loose effectively over time against a population of HIV virus in a patient. The reason being that under pressure of the presence of a particular HIV reverse transcriptase inhibitor, the existing population of HIV virus, usually mainly wild type HIV reverse transcriptase enzyme, mutates into different mutants which are far less sensitive to that same HIV reverse transcriptase inhibitor. If this phenomenon occurs, one talks about resistant mutants. If those mutants are not only resistant to that one particular HIV reverse transcriptase inhibitor, but also to multiple other commercially available HIV reverse transcriptase inhibitors, one talks about multi-drug resistant HIV reverse transcriptase. One way of expressing the resistance of a mutant to a particular HIV reverse transcriptase inhibitor is making the ratio between the EC₅₀ of said HIV reverse transcriptase inhibitor against mutant HIV reverse transcriptase over EC₅₀ of said HIV reverse transcriptase inhibitor against wild type HIV reverse transcriptase. Said ratio is also called fold change in resistance (FR). The EC₅₀ value represents the amount of the compound required to protect 50% of the cells from the cytopathogenic effect of the virus.

Many of the mutants occurring in the clinic have a fold resistance of 100 or more against the commercially available HIV reverse transcriptase inhibitors, like nevirapine, efavirenz, delavirdine. Clinically relevant mutants of the HIV reverse transcriptase enzyme may be characterized by a mutation at codon position 100, 103 and 181. As used herein a codon position means a position of an amino acid in a protein sequence. Mutations at positions 100, 103 and 181 relate to non-nucleoside RT inhibitors (D'Aquila et al. Topics in HIV medicine, 2002, 10, 11-15). Examples of such clinical relevant mutant HIV reverse transcriptases are listed in Table 1.

TABLE 1
List of mutations present in reverse transcriptase
of the HIV strains used.

| | |
|---|---|
| A | Y181C |
| B | K103N |
| C | L100I; K103N |
| D | L100I; K103N |
| E | F227C |
| F | Y188L |
| G | V106A, F227L |
| H | K103N, Y181C |
| I | K101E, K103N |
| J | I31L, L100I, K103N, E138G, Y181C, L214F |
| K | K2OR, E28K, M41L, E44A, D67N, L74I, K103N, V118I, |
| | D123N, S162C, Y181C, G196K, Q207E, L210W, L214F, |
| | T215Y, K219N, P225H, D250E, P272A, R277K, I293V, |
| | P297K, K311R, R358K, T376A, E399D, T400L |

An interesting group of compounds are those compounds of formula (I) having a fold resistance ranging between 0.01 and 100 against at least one mutant HIV reverse transcriptase, suitably ranging between 0.1 and 100, more suitably ranging between 0.1 and 50, and even more suitably ranging between 0.1 and 30. Of particular interest are the compounds of formula (I) showing a fold resistance against at least one mutant HIV reverse transcriptase ranging between 0.1 and 20, and even more interesting are those compounds of formula (I) showing a fold resistance against at least one mutant HIV reverse transcriptase ranging between 0.1 and 10.

An interesting group of compounds are those compounds of formula (I) having a fold resistance, determined according to the methods herein described, in the range of 0.01 to 100 against HIV species having at least one mutation in the amino acid sequence of HIV reverse transcriptase as compared to the wild type sequence (genbank accession e.g. M38432, K03455, gi 327742) at a position selected from 100, 103 and 181; in particular at least two mutations selected from the positions 100, 103 and 181. Even more interesting are those compounds within said interesting group of compounds having a fold resistance in the range of 0.1 to 100, in particular in the range 0.1 to 50, more in particular in the range 0.1 to 30. Most interesting are those compounds within said interesting group of compounds having a fold resistance in the range of 0.1 and 20, especially ranging between 0.1 and 10.

One embodiment relates to compounds of the present invention showing a fold resistance in the ranges mentioned hereinabove against at least one clinically relevant mutant HIV reverse transcriptase.

A particular subgroup of compounds are those compounds of formula (I) having an IC₅₀ of 1 μM or lower, suitably an IC₅₀ of 100 nM or lower vis-à-vis the wild type virus upon in vitro screening according to the methods described herein.

The ability of the present compounds to inhibit HIV-1, HIV-2, SIV and HIV viruses with reverse transcriptase (RT) enzymes having mutated under pressure of the currently known RT inhibitors, together with the absence of cross resistance with currently known RT inhibitors, indicate that the present compounds bind differently to the RT enzyme when compared to known NNRTIs and NRTIs. Other indicators of a different mode of action are the ribonucleotide sensitivity of the compounds of this invention as can be shown by their increased activity when administered in the presence of ATP and by their nucleoside competitive behaviour. The compounds of this invention therefore can be classified as nucleoside competitive reverse transcriptase inhibitors.

The compounds of the present invention show antiretroviral properties, in particular against Human Immunodeficiency Virus (HIV), which is the aetiological agent of Acquired Immune Deficiency Syndrome (AIDS) in humans. The HIV virus preferably infects CD4 receptor containing cells such as human T4 cells and destroys them or changes their normal function, particularly the coordination of the immune system. As a result, an infected patient has an ever-decreasing number of T4 cells, which moreover behave abnormally. Hence, the immunological defense system is unable to combat infections and/or neoplasms and the HIV infected subject usually dies by opportunistic infections such as pneumonia, or by cancers. Other diseases associated with HIV infection include thrombocytopaenia, Kaposi's sarcoma and infection of the central nervous system characterized by progressive demyelination, resulting in dementia and symptoms such as, progressive dysarthria, ataxia and disorientation. HIV infection further has also been associated with peripheral neuropathy, progressive generalized lymphadenopathy (PGL) and AIDS-related complex (ARC). The HIV virus also infects CD8-receptor containing cells. Other target cells for HIV virus include microglia, dendritic cells, B-cells and macrophages.

Due to these favourable pharmacological properties, the compounds of the present invention or any subgroup thereof may be used as a medicine against the above-mentioned diseases or in the prophylaxis thereof, or used in a method of treatment of the above-mentioned diseases or in the prophylaxis thereof. Said use as a medicine or method of treatment comprises the systemic administration to HIV-infected subjects, in particular human patients, of an amount of a compound of formula (I) or of a compound of a subgroup of compounds of formula (I), effective in the prophylaxis or treatment of the conditions associated with HIV infection.

In a further aspect, the present invention concerns the use of a compound of formula (I) or any subgroup thereof in the manufacture of a medicament useful for preventing, treating or combating infection or disease associated with HIV infection.

In another aspect, the present invention concerns the use of a compound of formula (I) or any subgroup thereof in the manufacture of a medicament useful for inhibiting replication of a HIV virus, in particular a HIV virus having a mutant HIV reverse transcriptase, more in particular having a multi-drug resistant mutant H reverse transcriptase.

In yet another aspect, the present invention relates to the use of a compound of formula (I) or any subgroup thereof in the manufacture of a medicament useful for preventing, treating or combating a disease associated with HIV viral infection wherein the reverse transcriptase of the HIV virus is mutant, in particular a multi-drug resistant mutant HIV reverse transcriptase.

The compounds of formula (I) or any subgroup thereof are also useful in a method for preventing, treating or combating infection or disease associated with HIV infection in a mammal, comprising administering to said mammal an effective amount of a compound of formula (I) or any subgroup thereof.

In another aspect, the compounds of formula (I) or any subgroup thereof are useful in a method for preventing, treating or combating infection or disease associated with infection of a mammal with a mutant HIV virus, comprising administering to said mammal an effective amount of a compound of formula (I) or any subgroup thereof.

In another aspect, the compounds of formula (I) or any subgroup thereof are useful in a method for preventing, treating or combating infection or disease associated with infection of a mammal with a multi drug-resistant HIV virus, comprising administering to said mammal an effective amount of a compound of formula (I) or any subgroup thereof.

In yet another aspect, the compounds of formula (I) or any subgroup thereof are useful in a method for inhibiting replication of a HIV virus, in particular a HIV virus having a mutant HIV reverse transcriptase, more in particular a multi-drug resistant mutant HIV reverse transcriptase, comprising administering to a mammal in need thereof an effective amount of a compound of formula (I) or any subgroup thereof.

Preferably, a mammal as mentioned in the methods of this invention is a human being.

The compounds of the present invention may also find use in inhibiting ex vivo samples containing HIV or expected to be exposed to HIV. Hence, the present compounds may be used to inhibit HIV present in a body fluid sample that contains or is suspected to contain or be exposed to HIV.

Particular reaction procedures to make the present compounds are described below. In the preparations described below, the reaction products may be isolated from the medium and, if necessary, further purified according to methodologies generally known in the art such as, for example, extraction, crystallization, trituration and chromatography.

The compounds of formula (I) wherein X is a group NR², which compounds may be represented by formula (I-b), can be prepared by N-alkylating intermediates of formula (II), with a suitable N-alkylating agent, as outlined in the following reaction scheme. The intermediates of formula (II-a) are analogs of the compounds of formula (I) wherein the R² substituent is hydrogen.

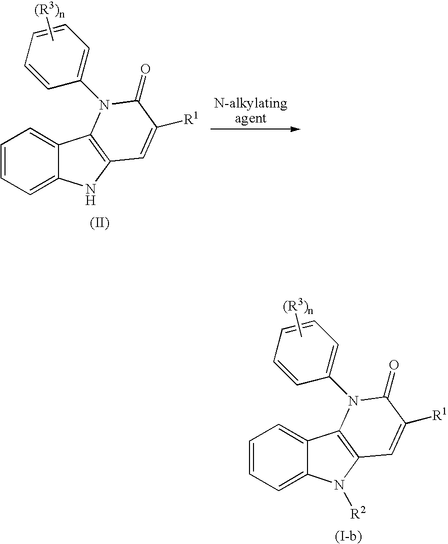

In one embodiment, the N-alkylating reagent is a reagent, which can be represented by formula R²—W (III-a), wherein W is a leaving group. Suitable leaving groups are halo, in particular chloro, bromo and iodo, or other leaving groups such as for example sulfonates, e.g. tosylates, mesylates and the like. This type of N-alkylation reaction may be performed in an appropriate solvent in the presence of a suitable base such as a alkali metal hydride, e.g. sodium or potassium hydride, or an alkali or each alkaline metal hydroxide, carbonate or hydrogencarbonate, e.g. sodium or potassium carbonate, sodium or potassium hydroxide, calcium hydroxide, sodium or potassium hydrogencarbonate and the like.

Some of the compounds of formula (I-b) may also, where appropriate, be prepared by a reductive amination reaction which comprises reacting intermediates (II-a) with an intermediate R^{2-a}═O (III-b), wherein R^{2-a} has the same meanings of R² provided that it has a carbon atom that can form an aldehyde of ketone functionality. This reaction may be conducted in the presence of hydrogen and a suitable catalyst, in particular a noble metal catalyst such as Pd or Pt, usually in a suitable solvent such as an ether or alcohol.

Some of the R² groups may also be introduced using R² groups derived from an epoxide. This type of reaction is particularly suited for introducing R² groups wherein R² is a radical (b-3), (b-4) or (b-5).

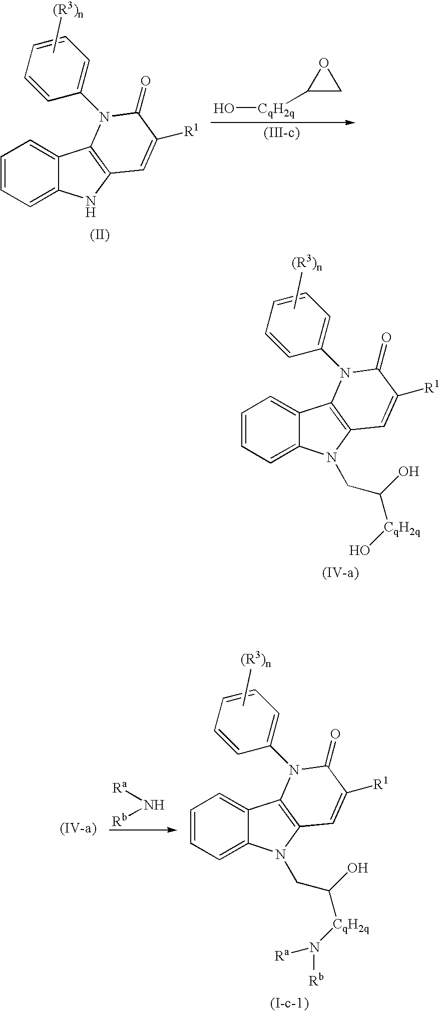

For example, compounds of formula (I-b), wherein R² is a radical (b-3) wherein p is 1 and wherein the group —NR^{a}R^{b} are certain radicals amongst R¹⁵ such as —NR^{16a}R^{16b}, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl, pyrrolyl, oxazolyl, thiazolyl, imidazolyl, isoxazolyl, isothiazolyl, pyrazolyl, oxadiazolyl, thiadiazolyl, triazolyl, tetrazolyl, a radical (a-1), (a-2), (a-3), or (a-5); wherein any of the foregoing heterocycles such as pyrrolidinyl, piperidinyl, homopiperidinyl, etc. is substituted on the C_{q}H_{2q} moiety via a nitrogen atom; which compounds can be represented by formula (I-c-1); can be prepared by reacting an intermediate of formula (II-a) wherein R² is hydrogen with an epoxide of formula (III-c). The resulting intermediates of formula (IV-a) can be converted into compounds of formula (I-c-1) wherein —NR^{a}R^{b} is as specified above by an appropriate alcohol (C—OH) to amine (C—N) conversion reaction. The alcohol group may be converted into a suitable leaving group and subsequently reacted with an amine H—NR^{a}R^{b}. In an alternative execution, the alcohol group may be converted to an amine bond by a Mitsonobu-type reaction using an azodicarboxylate/triphenyl phosphine reagent, for example diisopropylazodicarboxylate (DIAD), and subsequent reaction with the appropriate amine. The thus obtained compounds of formula (I-c-1) can be O-alkylated or O-acylated in order to obtain the analogs of the compounds (I-c-1) wherein R¹⁴ is other than hydrogen.

In a similar process, intermediates (II) are reacted with a epoxide (III-d) using a hydroxyl to amino conversion reaction such as the above describe Mitsonobu reaction to obtain an epoxide (IV-b), The latter is reacted with an amine to yield compounds of formula (I-c-2) as outlined in the following reaction scheme. The compounds of formula (I-c-2) can also be O-alkylated or O-acylated as described in the previous paragraph.

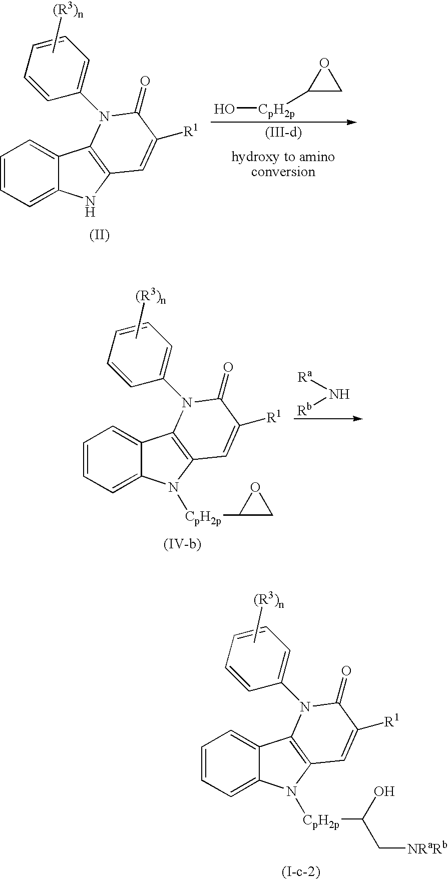

In an alternative procedure, intermediate (II-a) can be reacted with an epoxide having formula 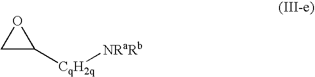
to directly obtain compounds of formula (I) wherein R² is a radical (b-3) wherein R¹⁵ is an amino substituent —N^{a}R^{b}.

The intermediates of formula (IV-b) can also be reacted with an alkanolamine to obtain compounds of formula (I-c-3), which are cyclized to obtain compounds (I-c-4), which are compounds of formula (I) wherein R² is alkyl substituted with a radical of formula (a-4). The cyclization may be conducted in the presence an acid such as hydrochloric acid with removal of water or in the presence of a suitable dehydrating agent for example sulfonyl amide such as an arylsulfonyl imidazole. These reactions are represented in the following reaction scheme wherein R^{a} has the meanings of R^{13a}, provided that it is other than hydrogen. R^{a} can also be a N-protecting group which is removed afterwards, thus giving access to compounds wherein R^{13a} is hydrogen.

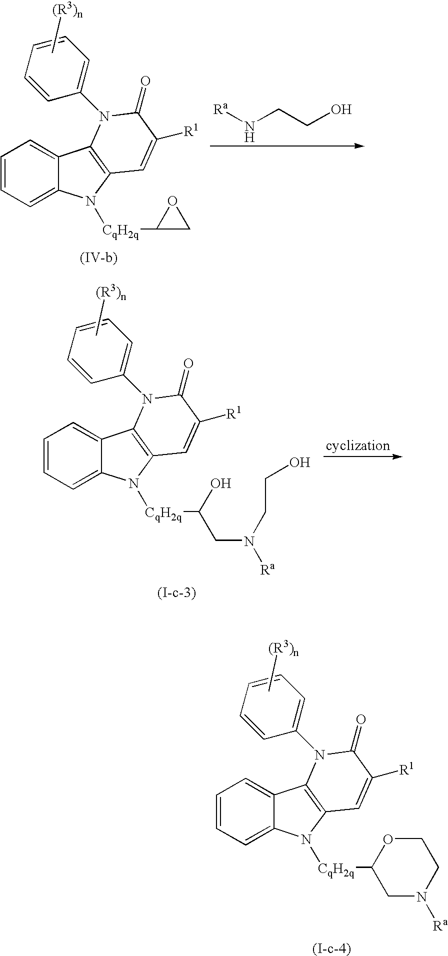

Compounds of formula (I-d) which are compounds of formula (I) wherein R² is a group (b-4) wherein R¹⁴ is hydrogen can be prepared starting from intermediates of formula (II) which are reacted with ethylene oxide to obtain intermediates of formula (V), followed by controlled addition of further ethylene oxide moieties as outlined in the following reaction scheme.

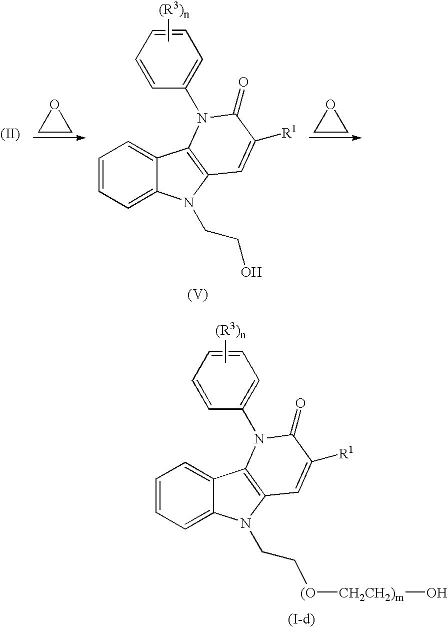

The resulting compounds (1-d) may be alkylated to yield compounds of formula (I) having a (b-4) group with a R¹⁴ radical that is other than hydrogen. Or the compounds (I-d) may be converted into the corresponding amines (b-5) using a suitable alcohol to amine conversion reaction.

A further aspect of this invention concerns the fact that the intermediates of formula (IV-a), (IV-b) and (V) are novel compounds. The intermediates of formula (IV-a) and (V) have been found to possess similar HIV-inhibiting properties as the compounds of formula (I). Thus in a further aspect, the invention provides compounds of formula (IV-a) or (IV-b), or the acid-addition salts thereof, or the stereoisomers thereof, having the structural formulae depicted above. The acid-addition salts are the same as those described in relation to the compounds of formula (I). Preferred are the pharmaceutically acceptable acid-addition salts. The intermediates of formula (IV-a) and (V) may be formulated into suitable pharmaceutical formulations, and they may be used in similar uses and methods, as described for the compounds of formula (I).

Compounds of formula (I) wherein R² is a phenyl group substituted with a —COOR⁴ group can be obtained by a suitable N-arylation reaction. In this reaction procedure, an intermediate (II-a) is reacted with a suitably substituted aryl group.

Compounds wherein R² is a group (b-1) can be prepared starting from a pyrrolidine, piperidine or homopiperidine derivative having a suitable leaving group. Similarly compounds wherein R² is a group (b-2) can be prepared starting from a morpholine having a suitable leaving group. If necessary, the nitrogen atom in the a pyrrolidine, piperidine, homopiperidine or morpholine groups may be protected by a suitable N-protecting group (e.g. benzyl, benzyloxycarbonyl, t.butyloxycarbonyl, etc.) which subsequently is removed.

Compounds of formula (I-b) wherein R² is C₁₋₁₀alkyl, C₂₋₁₀alkenyl, C₃₋₇cycloalkyl, substituted with a radical selected from —NR⁷R⁸, —NR⁹R¹⁰, a radical (a-1), (a-2), (a-3) or (a-5), as specified above, can be prepared starting from an intermediate (II) which is reacted with a C₁₋₁₀alkane, C₂₋₁₀alkenyl or C₃₋₇cycloalkane bearing two leaving groups in a controlled manner such that only one of the leaving groups is substituted. Subsequently the thus obtained intermediate is reacted with an appropriate amine thus substituting the second leaving group. For example (II) may be reacted with a C₁₋₁₀alkanediyl dihalide and subsequently reacted with an amine H—NR⁷R⁸, H—NR⁹R¹⁰ or another amine. Other similar process variants may be used in which some or several functionalities are protected and subsequently deprotected.

The compounds of formula (I) wherein X is O, wherein R² is cyano, which compounds are represented by formula (I-d), can be prepared as outlined in the following reaction scheme.

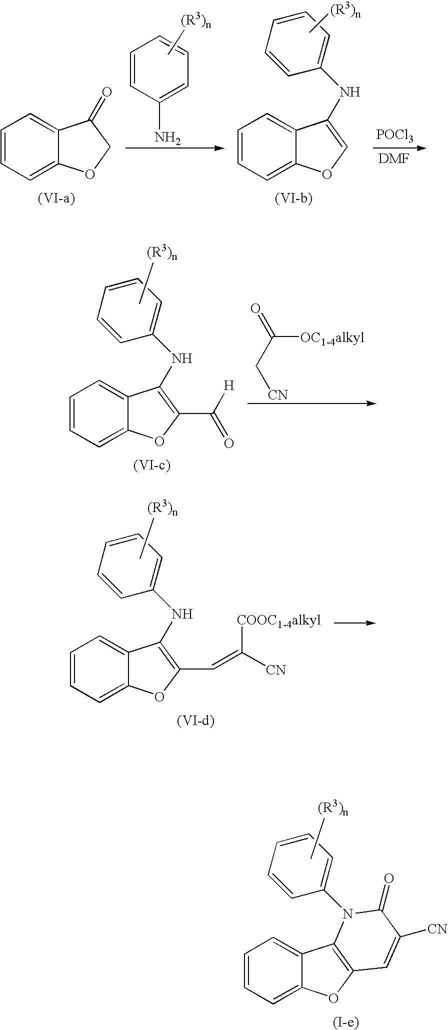

The intermediate 3-hydroxybenzofuran (VI-a) is condensed with a suitable aniline derivative to result in a 3-phenylaminobenzofuran (VI-b) [V. A. Azimov, S. Yu. Ryabova, L. M. Alekseeva and V. G. Granik, Chemistry of heterocyclic compounds 2000, 36, 1272-1275]. The conversion from (VI-a) to (VI-b) typically is conducted in a suitable solvent such as a hydrocarbon, for example toluene, typically in the presence of a catalytic amount of acid such as e.g. p-toluenesulfonic acid. The 3-phenylaminobenzofuran (VI-b) is formylated, for example by using phosphorus oxychloride in DMF followed by hydrolysis. The formylated derivative (VI-c) may be converted to a compound (VI-d) by using a cyano acetate derivative, typically in a suitable solvent such as an alcohol e.g. iso-propanol, in the presence of a base, preferably a tertiary amine base such as triethylamine. Intermediate (VI-d) subsequently is cyclized at elevated temperature to yield a compound (VI-e). A suitable solvent for this cyclization reaction is a glycol such as ethylene glycol.

This synthesis route may also be used to prepare analogs of the compounds (I-e) wherein R¹ is other than cyano, in particular those compounds (I-e) wherein R¹ is C₁₋₄alkyloxycarbonyl, by reacting (VI-c) with a di(C₁₋₄alkyl)malonic acid ester.

The compounds of formula (I) wherein X is S can be prepared from the sulfur analogs of intermediate (VI-a), i.e. 3-hydroxybenzothiene, following the same procedures outlined above yielding the sulfur analogs of compounds (I-e). The latter can be converted to the corresponding sulfoxides (X is SO) or sulfones (X is SO₂) using art known oxidation procedures, e.g. by treatment with a suitable peroxide.

The compounds of formula (I) may be transferred into other compounds of formula (I) with different substitution using art-known transformation techniques. For instance, the compounds of formula (I) wherein R³ is nitro may be reduced to R³ being amino, and may then be further derivatized. Further examples of transformation reactions are given in the experimental part.

The compounds of formula (I) wherein R¹ is cyano may be hydrolysed to the corresponding compounds of formula (I) wherein R¹ is hydroxycarbonyl, which in turn may be esterified to obtain compounds of formula (I) wherein R¹ is C₁₋₄alkyloxycarbonyl. The latter or the hydroxycarbonyl derivatives may be converted to the corresponding amides using art-known carboxyl to amide or alkylester to amide transformation reactions.

Compounds of formula (I) having a —COOR⁴ group wherein R⁴ is hydrogen may be converted to the corresponding esters using art-known esterification procedures. Vice versa, the esters can be converted to the free acid by suitable hydrolysis procedures, e.g. by hydrolysis in acidic or basic media.

Compounds of formula (I) having a thiomorpholinyl group can be oxidized to the corresponding 1-oxothiomorpholinyl or 1,1-dioxothiomorpholinyl containing compounds using a suitable organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chloro-benzenecarboperoxoic acid, peroxoalkanoic acids, e.g. peroxoacetic acid, alkylhydroperoxides, e.g. tert-butyl hydroperoxide. The 1-oxothiomorpholinyl analogs are preferably obtained using controlled oxidation procedures.

The compounds of formula (I) may also be converted to the corresponding N-oxide forms following art-known procedures for converting a tri-substituted nitrogen into its N-oxide form. Said N-oxidation reaction may generally be carried out by reacting the starting material of formula (I) with a suitable organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chloro-benzenecarboperoxoic acid, peroxoalkanoic acids, e.g. peroxoacetic acid, alkylhydroperoxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

A basic nitrogen occurring in the present compounds can be quaternized with any agent known to those of ordinary skill in the art including, for instance, lower alkyl halides, dialkyl sulfates, long chain halides and aralkyl halides according to art-known procedures.

A number of the intermediates used to prepare the compounds of formula (I) are known compounds while others are analogs of known compounds which can be prepared following modifications of art-known methodologies readily accessible to the skilled person. A number of preparations of intermediates are given hereafter in somewhat more detail. In the following reaction schemes the radicals R¹, R², R³, n have the meanings specified in relation to the compounds of formula (I) or any of the subgroups of compounds of formula (I). W represents a leaving group such as tosyl, mesyl, halo, in particular chloro or bromo.

The intermediates of formula (II) can be prepared as outlined in the following reaction scheme.

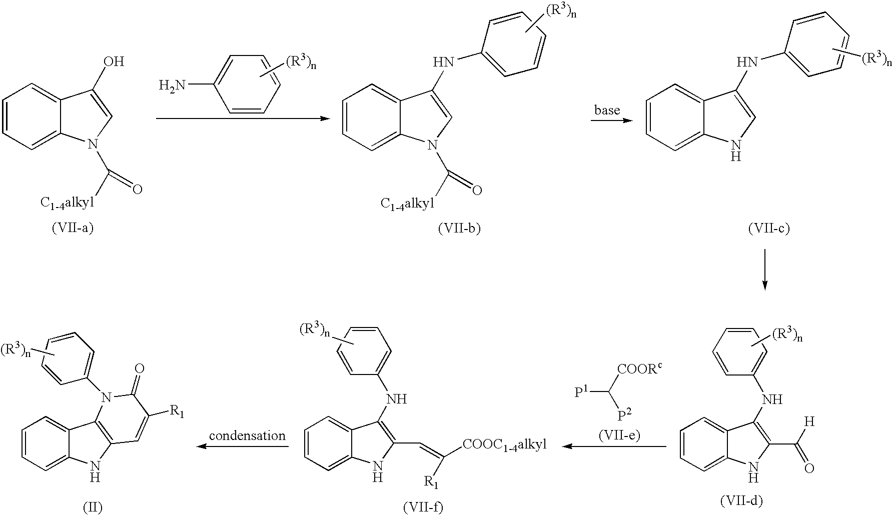

The synthesis of intermediates (II) starts from a 1-C₁₋₄alkylcarbonyl-3-hydroxy-indole (VII-a) which is condensed with a substituted aniline, yielding 3-(phenylamino)indoles (VII-b). This condensation reaction may be conducted at elevated temperatures and in acidic circumstances, e.g. by using an acidic solvent such as acetic acid, or using a solvent such as toluene, benzene, an alcohol and the like, in the presence of a suitable acid catalyst such as p-toluene sulfonic acid. Intermediate (VII-b) subsequently is deacylated with a base, such as for example triethylamine, sodium or potassium hydroxide, sodium acetate, potassium acetate or potassium carbonate and the like, in a suitable solvent, such as for example methanol or ethanol, preferably at elevated temperature, yielding intermediates (VII-c). Formylation of intermediate (VII-c), for instance by applying a Vilsmeier reaction, results in indole aldehydes (VII-d). Condensation of intermediates (VII-d) with a reagent (VII-e) results in intermediate (VII-f). The radicals P¹, P² and R^{c} in (VII-e) may have various meanings depending on the type of reaction used to obtain the intermediates (VII-f). In one embodiment, this condensation may be performed in a Knoevenagel type opf reaction with an substituted acetic acid ester of formula R¹—CH₂—COOR^{c} (which is an intermediate (VII-e) wherein P¹ is R¹, P² is H and R^{c} is C₁₋₆alkyl or arylC₁₋₆alkyl), using a base such as for example triethylamine, sodium acetate, potassium acetate, piperidine and the like, in a wide variety of solvents. Alternatively use may be made of a Wittig reaction or a Wittig-Horner reaction. In the former instance a Wittig type reagent, such as a triphenyl-phosphoniumylide is used. The Wittig conversion is conducted in a suitable reaction-inert solvent such as an ether, starting from triphenylphosphine and a halo acitic acid ester of formula R¹CH(Halo)-COOR^{4a}. The Wittig-Horner reaction is performed using a phosphonate, such as e.g. a reagent of formula di(C₁₋₆-alkyloxy)-P(═O)—CH(R¹)—COOR^{4a} in the presence of a base, preferably a strong base, in an aprotic organic solvent. Subsequent cyclisation of intermediate (VII-f) at elevated temperature and in a solvent like ethylene glycol, dioxane, N,N-dimethylformamide, dimethylsulfoxide, glyme, diglyme and the like, yields intermediates (II).

The order of the reaction steps in the process set out in the above reaction scheme may be different. For instance the formylation may be performed prior to deacylation.

This synthesis pathway is particularly useful for preparing intermediates of formula (II) wherein R¹ is cyano. It may also be used to prepare intermediates wherein R¹ is aminocarbonyl, C₁₋₄alkyloxycarbonyl, mono- or di(C₁₋₄alkyl)aminocarbonyl, arylaminocarbonyl, N-(aryl)-N—(C₁₋₄alkyl)aminocarbonyl, Het₁ or Het₂. The intermediates of formula (II) obtained through this reaction pathway may be converted to analogous intermediates of formula (II) wherein R¹ has the other meanings by functional group transformation reactions such as cyano to carboxyl hydrolysis, carboxyl to amide conversion, etc.

This synthesis pathway moreover is particularly useful to prepare intermediates of formula (II) wherein RT is nitro or cyano. In one embodiment, R³ is para-nitro and the process starts from para-nitroaniline.

The intermediates of formula (II-a), which are intermediates of formula (II) wherein R¹ is cyano, may alternatively be prepared as outlined in the following reaction scheme.

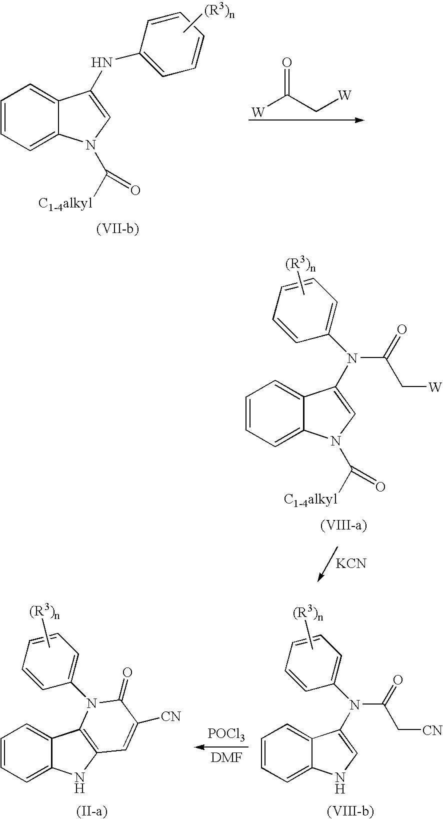

Intermediate (VII-b), which is prepared as described in the previous reaction scheme, is reacted with chloroacetyl chloride or a functional derivative thereof, suitably at elevated temperature, to yield an intermediate of formula (VIII-a). The latter intermediate of formula (VIII-a) is deprotected using a suitable base such as trietylamine, sodium acetate, potassium acetate, sodium hydroxide, potassium hydroxide, potassium carbonate and the like, in a solvent like methanol or ethanol. The thus formed intermediate (VIII-b) is converted to the corresponding cyano derivative (VIII-b) using potassium cyamide or tetrabutylammoniumcyanide. The cyano derivative (VIII-b) is cyclized in a two step procedure comprising first a Vilsmeier formylation using POCl₃ in N,N-dimethylformamide and subsequent cyclization to form intermediate (II-a).

Intermediates of formula (II-b), which are intermediates of formula (II) wherein R¹ is hydrogen, can be prepared as outlined in the following reaction scheme.

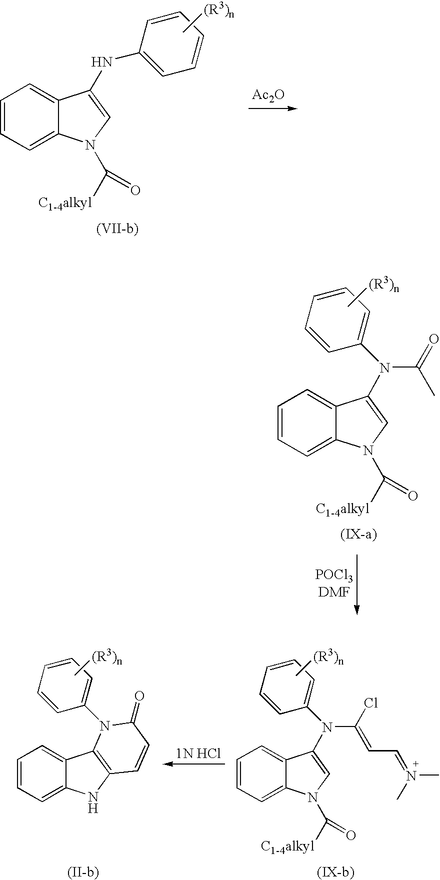

This synthesis pathway is particularly useful for preparing compounds of formula (I) wherein R³ is cyano, nitro or C₁₋₆alkyloxycarbonyl.

Intermediate (VII-b), which is prepared as outlined above, is reacted with acetic anhydride in the presence of a catalyst such as for example pyridine or dimethylaminopyridine or the like, suitably at elevated temperature, to yield an intermediate of formula (IX-a). The thus formed intermediate of formula (IX-a) is formylated using a Vilsmeier reaction with POCl₃ in N,N-dimethylformamide, to form intermediate (IX-b) which in turn can be further cyclized to intermediates (II-b), e.g. in an aqueous acidic environment, e.g. in aqueous HCl.

Intermediates of formula (II-a) or (II-b) may be transformed into other intermediates of formula (II) using art-known functional group transformation reactions. For example where R³ is Br, Br may be transformed into a heterocyclic ring using heterocyclic borates and palladium. Or where R³ is C₁₋₆alkyloxycarbonyl this radical may be transformed to the equivalent carboxylic acid or amide using a hydrolysis reaction, or respectively, an ester or carboxylic acid to amide reaction. Also R³ being cyano may be transformed to a heterocycle such as a tetrazolyl, oxadiazolyl, thiazolyl etc. using art-known cyclization procedures.

The compounds of the present invention may be used in animals, preferably in mammals, and in particular in humans as pharmaceuticals per se, in mixtures with one another or in the form of pharmaceutical preparations.

Consequently, the present invention relates to pharmaceutical formulations containing as active ingredients an effective dose of at least one of the compounds of formula (I) in addition to customary pharmaceutically innocuous excipients and auxiliaries. The pharmaceutical preparations may contain 0.1 to 90% by weight of a compound of formula (I). The pharmaceutical preparations can be prepared in a manner known per se to one of skill in the art. For this purpose, a compound of formula (I), together with one or more solid or liquid pharmaceutical excipients and/or auxiliaries and, if desired, in combination with other pharmaceutical active compounds, are brought into a suitable administration form or dosage form which can then be used as a pharmaceutical product in human medicine or veterinary medicine.

Pharmaceuticals which contain a compound according to the invention can be administered orally, parenterally, e.g., intravenously, rectally, by inhalation, or topically, the preferred route of administration being dependent on the individual case, e.g., the particular course, of the disorder to be treated. Oral administration is preferred.

The person skilled in the art is familiar on the basis of his expert knowledge with the auxiliaries that are suitable for the desired pharmaceutical formulation. Beside solvents, gel-forming agents, suppository bases, tablet auxiliaries and other active compound carriers, antioxidants, dispersants, emulsifiers, antifoams, flavor corrigents, preservatives, solubilizers, agents for achieving a depot effect, buffer substances or colorants are also useful.

Also, the combination of an antiretroviral compound and a compound of the present invention can be used. Thus, to prevent, combat or treat HIV infections and the diseases associated with HIV infection, such as Acquired Immunodeficiency Syndrome (AIDS) or AIDS Related Complex (ARC), the compounds of this invention may be co-administered in combination with for instance, binding inhibitors, fusion inhibitors, co-receptor binding inhibitors; RT inhibitors; nucleoside RTIs; nucleotide RTIs; NNRTIs; RNAse H inhibitors; TAT inhibitors; integrase inhibitors; protease inhibitors; glycosylation inhibitors; entry inhibitors.

Any of these combinations may provide a synergistic effect, whereby viral infectivity and its associated symptoms may be prevented, substantially reduced, or eliminated completely.

Thus in a further aspect, the present invention also relates to combinations containing:
(a) a compound of the present invention, in particular a compound of formula (I) as defined herein, or a compound of formula (I) of any of the subgroups specified herein; an N-oxide, salt, stereoisomeric form, prodrug, ester or metabolite thereof, and
(b) another anti-retroviral compound, in particular another HIV inhibitor.

The present invention additionally relates to combinations containing
(a) a compound of the present invention, in particular a compound of formula (I) as defined herein, or a compound of formula (I) of any of the subgroups specified herein, an N-oxide, salt, stereoisomeric form, prodrug, ester or metabolite thereof, and
(b) any of the agents selected from binding inhibitors, such as, for example, dextran sulfate, suramine, polyanions, soluble CD4, PRO-542, BMS-806; fusion inhibitors, such as, for example, T20, T1249, RPR 103611, YK-FH312, IC 9564, 5-helix, D-peptide ADS-J1; co-receptor binding inhibitors, such as, for example, AMD 3100, AMD-3465, AMD7049, AMD3451 (Bicyclams), TAK 779, T-22, ALX40-4C; SHC-C (SCH351125), SHC-D, PRO-140, RPR103611; RT inhibitors, such as, for example, foscarnet and prodrugs; nucleoside RTIs, such as, for example, AZT, 3TC, DDC, DDI, D4T, Abacavir, FTC, DAPD (Amdoxovir), dOTC (BCH-10652), fozivudine, DPC 817; nucleotide RTIs, such as, for example, PMEA, PMPA (tenofovir); NNRTIs, such as, for example, nevirapine, delavirdine, efavirenz, 8 and 9-Cl TIBO (tivirapine), loviride, TMC-125, dapivirine, MKC-442, UC 781, UC 782, Capravirine, QM96521, GW420867X, DPC 961, DPC963, DPC082, DPC083, calanolide A, SJ-3366, TSAO, 4″-deaminated TSAO, MV150, MV026048, PNU-142721; RNAse H inhibitors, such as, for example, SP1093V, PD126338; TAT inhibitors, such as, for example, RO-5-3335, K12, K37; integrase inhibitors, such as, for example, L 708906, L 731988, S-1360; protease inhibitors, such as, for example, amprenavir and fosamprenavir, ritonavir, nelfinavir, saquinavir, indinavir, lopinavir, palinavir, BMS 186316, atazanavir, DPC 681, DPC 684, tipranavir, AG1776, mozenavir, DMP-323, GS3333, KNI-413, KNI-272, L754394, L756425, LG-71350, PD161374, PD173606, PD177298, PD178390, PD178392, PNU 140135, TMC-114, maslinic acid, U-140690; glycosylation inhibitors, such as, for example, castanospermine, deoxynojirimycine; entry inhibitors CGP64222; hereafter referred to as agents belonging to group (b).

In one embodiment there are provided combinations containing ingredients (a) and (b), as specified above, wherein the compound of the present invention is a compound (I-a), an N-oxide, salt, stereoisomeric form, prodrug, ester or metabolite thereof.

In another embodiment there are provided combinations containing ingredients (a) and (b), as specified above, wherein the compound of the present invention is selected from the group consisting of:
1-(4-Nitro-phenyl)-2-oxo-1,2-dihydro-benzo[4,5]furo[3,2-b]pyridine-3-carbonitrile,
5-(2-Hydroxy-3-morpholin-4-yl-propyl)-1-(4-nitro-phenyl)-2-oxo-2,5-dihydro-1H-pyrido[3,2-b]indole-3-carbonitrile,
5-(2-Hydroxy-3-piperidin-1-yl-propyl)-1-(4-nitro-phenyl)-2-oxo-2,5-dihydro-1H-pyrido[3,2-b]indole-3-carbonitrile,
5-(3-Diethylamino-2-hydroxy-propyl)-1-(4-nitro-phenyl)-2-oxo-2,5-dihydro-1H-pyrido[3,2-b]indole-3-carbonitrile,
5-[2-(2-Methoxy-ethoxy)-ethyl]-1-(4-nitro-phenyl)-2-oxo-2,5-dihydro-1H-pyrido-[3,2-b]indole-3-carbonitrile, and especially
5-(2-Hydroxy-3-pyrrolidin-1-yl-propyl)-1-(4-nitro-phenyl)-2-oxo-2,5-dihydro-1H-pyrido[3,2-b]indole-3-carbonitrile,
   and their N-oxides, salts and possible stereoisomers, said group hereafter being referred to as “group of compounds (I-f)”.

Embodiments of this invention are combinations comprising (a) one or more compounds of formula (I), or compounds of any of the subgroups of compounds of formula (I), as specified herein, in particular of the subgroups of compounds of formula (I-a), or the group of compounds (I-f), including the N-oxides, salts, stereoisomeric forms, prodrugs, esters and metabolites thereof, and (b) one or more HIV inhibitors selected from:
(i) one or more fusion inhibitors, such as, for example, T20, T1249, RPR 103611, YK-FH312, IC 9564, 5-helix, D-peptide ADS-J1, enfuvirtide (ENF), GSK-873,140, PRO-542, SCH-417,690. TNX-355, maraviroc (UK-427,857); preferably one or more fusion inhibitors, such as, for example, enfuvirtide (ENF), GSK-873,140, PRO-542, SCH-417,690. TNX-355, maraviroc (UTK-427,857);
(ii) one or more nucleoside RTIs, such as for example AZT, 3TC, zalcitabine (ddC), ddI, d4T, Abacavir (ABC), FTC, DAPD (Amdoxovir), dOTC (BCH-10652), fozivudine, D-D4FC (DPC 817 or Reverset™), alovudine (MIV-310 or FLT), elvucitabine (ACH-126,443); preferably one or more nucleoside RTIs, such as for example, AZT, 3TC, zalcitabine (ddC), ddI, d4T, Abacavir (ABC), FTC, DAPD (Amdoxovir), D-D4FC (DPC 817 or Reverset™), alovudine (MIV-310 or FLT), elvucitabine (ACH-126,443);
(iii) nucleotide RTIs, such as, for example, PMEA, PMPA (TDF or tenofovir) or tenofovir disoproxil fumarate; preferably tenofovir or tenofovir disoproxil fumarate;
(iv) one or more NNRTIs such as, for example, nevirapine, delavirdine, efavirenz, 8 and 9-Cl TIBO (tivirapine), loviride, TMC125, 4-[[4-[[4-(2-cyanoethenyl)-2,6-diphenyl]amino]-2-pyrimidinyl]amino]-benzonitrile (TMC278 or R278474), dapivirine (R147681 or TMC120), MKC-442, UC 781, UC 782, Capravirine, QM96521, GW420867X, DPC 961, DPC963, DPC082, DPC083 (or BMS-561390), calanolide A, SJ-3366, TSAO, 4″-deaminated TSAO, MV150, MV026048, PNU-14272; or preferably one or more NNRTIs such as for example nevirapine, delavirdine, efavirenz, TMC125, TMC278, TMC120, capravirine, DPC083, calanolide A;
(v) one or more protease inhibitors, such as, for example, amprenavir and fosamprenavir, lopinavir, ritonavir (as well as combinations of ritonavir and lopinavir such as Kaletra™), nelfinavir, saquinavir, indinavir, palinavir, BMS 186316, atazanavir, DPC 681, DPC 684, tipranavir, AG1776, mozenavir, DMP-323, GS3333, KNI-413, KNI-272, L754394, L756425, LG-71350, PD161374, PD173606, PD177298, PD178390, PD178392, PNU 140135, TMC-114, maslinic acid, U-140690; in particular one or more protease inhibitors, such as, for example, amprenavir and fosamprenavir, lopinavir, ritonavir (as well as combinations of ritonavir and lopinavir), nelfinavir, saquinavir, indinavir, atazanavir, tipranavir, TMC-114.

In a further aspect the present invention provides combinations comprising at least one compound of formula (I) or compounds of any of the subgroups of compounds of formula (I), as specified herein, in particular of the subgroups of compounds of formula (I-a), or the group of compounds (I-f), including the N-oxides, salts, stereoisomeric forms, prodrugs, esters and metabolites thereof, and at least two different other antiretroviral agents.

One embodiment are combinations as specified in the previous paragraph wherein said two different other antiretroviral agents are

(i) two nucleoside transcriptase inhibitors (NRTIs);

(ii) a nucleoside (NRTIs) and a nucleotide reverse transcriptase inhibitor (NtRTI);

(iii) an NRTI and an NNRTI;

(iv) an NRTI and a protease inhibitor (PI);

(v) two NRTIs and a PI;

(vi) an NRTI and a fusion inhibitor.

The NRTIs, NtRTIs, NNRTIs, PIs and fusion inhibitors in the combinations mentioned in the previous paragraph may be selected from the groups of NRTIs, NtRTIs, NNRTIs, PIs and fusion inhibitors (i), (ii), (iii), (iv) or (v) mentioned above in relation to embodiments which are combinations comprising ingredients (a) and (b).

Of particular interest among the combinations mentioned above are those comprising a compound of the present invention having the formula (I) or (I-a), or belonging to or the group of compounds (I-f), as specified above, and:
(1) a fusion inhibitor selected from enfuvirtide (ENF), GSK-873,140, PRO-542, SCH-417,690. TNX-355, maraviroc (UK-427,857);
(2) an NNRTI selected from nevirapine, delavirdine, efavirenz, TMC125, TMC278, TMC120, capravirine, DPC083, calanolide A;
(3) an NRTI selected from AZT, 3TC, zalcitabine (ddC), ddI, d4T, Abacavir (ABC), FTC, DAPD (Amdoxovir), D-D4FC (DPC 817 or Reverset™), alovudine (MIV-310 or FLT), elvucitabine (ACH-126,443).
(4) an NtRTI selected from tenofovir or tenofovir disoproxil fumarate;
(5) a PI selected from amprenavir and fosamprenavir, lopinavir, ritonavir (as well as combinations of ritonavir and lopinavir), nelfinavir, saquinavir, indinavir, atazanavir, tipranavir, TMC-114;
(6) a NRTI as in (3) and a PI as in (5);
(7) two different NRTIs as in (3);
(8) an NRTI as in (3) and an NNRTI as in (2);
(9) two different NRTIs as in (3) and an NNRTI as in (2);
(10) two different NRTIs as in (3) and a PI as in (5);
(11) a NRTI as in (3) and an NtRTI as in (4); or
(12) a NRTI and a fusion inhibitor as in (1).

One type of embodiments of this invention are those combinations as outlined herein that do not contain 3TC.

The present invention also relates to a product containing (a) a compound of the present invention, in particular a compound of formula (I) as defined herein, or a compound of formula (I) of any of the subgroups defined herein, its N-oxides, salts, stereoisomeric forms, prodrugs, esters and metabolites, or any compound of a subgroup as specified herein, and (b) another antiretroviral compound, as a combined preparation for simultaneous, separate or sequential use in treatment of retroviral infections such as HIV infection, in particular, in the treatment of infections with multi-drug resistant retroviruses.

Any of the above combinations may provide a synergistic effect, whereby viral infectivity and its associated symptoms may be prevented, substantially reduced, or eliminated completely.

Any of the above mentioned combinations or products may be used to prevent, combat or treat HIV infections and the disease associated with HIV infections, such as Acquired Immunodeficiency Syndrome (AIDS) or AIDS Related Complex (ARC). Therefore in a further aspect there are provided methods of treating mammals, in particular humans, being infected with HIV or at risk of being infected with HIV, said method comprising administering to said mammals, or in particular to said humans, a combination or a product as specified herein.

The compounds of the present invention may also be administered in combination with immunomodulators (e.g., bropirimine, anti-human alpha interferon antibody, IL-2, methionine enkephalin, interferon alpha, and naltrexone) with antibiotics (e.g., pentamidine isothiorate) cytokines (e.g. Th2), modulators of cytokines, chemokines or modulators of chemokines, chemokine receptors (e.g. CCR5, CXCR4), modulators chemokine receptors, or hormones (e.g. growth hormone) to ameliorate, combat, or eliminate HIV infection and its symptoms. Such combination therapy in different formulations, may be administered simultaneously, sequentially or independently of each other. Alternatively, such combination may be administered as a single formulation, whereby the active ingredients are released from the formulation simultaneously or separately.

The compounds of the present invention may also be administered in combination with modulators of the metabolization following application of the drug to an individual. These modulators include compounds that interfere with the metabolization at cytochromes, such as cytochrome P450. It is known that several isoenzymes exist of cytochrome P450, one of which is cytochrome P450 3A4. Ritonavir is an example of a modulator of metabolization via cytochrome P450. Such combination therapy in different formulations, may be administered simultaneously, sequentially or independently of each other. Alternatively, such combination may be administered as a single formulation, whereby the active ingredients are released from the formulation simultaneously or separately. Such modulator may be administered at the same or different ratio as the compound of the present invention. Preferably, the weight ratio of such modulator vis-à-vis the compound of the present invention (modulator:compound of the present invention) is 1:1 or lower, more preferable the ratio is 1:3 or lower, suitably the ratio is 1:10 or lower, more suitably the ratio is 1:30 or lower.

For an oral administration form, compounds of the present invention are mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms.

For subcutaneous or intravenous administration, the active compounds, if desired with the substances customary therefore such as solubilizers, emulsifiers or further auxiliaries, are brought into solution, suspension, or emulsion. The compounds of formula (I) can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned.

Suitable pharmaceutical formulations for administration in the form of aerosols or sprays are, for example, solutions, suspensions or emulsions of the compounds of formula (I) or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Such a preparation customarily contains the active compound in a concentration from approximately 0.1 to 50%, in particular from approximately 0.3 to 3% by weight.

In order to enhance the solubility and/or the stability of the compounds of formula (I) in pharmaceutical compositions, it can be advantageous to employ α-, β- or γ-cyclo-dextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

Appropriate cyclodextrins are α-, β- or γ-cyclodextrins (CDs) or ethers and mixed ethers thereof wherein one or more of the hydroxy groups of the anhydroglucose units of the cyclodextrin are substituted with C₁₋₆alkyl, particularly methyl, ethyl or isopropyl, e.g. randomly methylated β-CD; hydroxyC₁₋₆alkyl, particularly hydroxyethyl, hydroxypropyl or hydroxybutyl; carboxyC₁₋₆alkyl, particularly carboxymethyl or carboxyethyl; C₁₋₆alkyl-carbonyl, particularly acetyl; C₁₋₆alkyloxycarbonylC₁₋₆alkyl or carboxyC₁₋₆alkyloxyC₁₋₆alkyl, particularly carboxymethoxy-propyl or carboxyethoxy-propyl; C₁₋₆alkylcarbonyloxyC₁₋₆alkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD).

The term mixed ether denotes cyclodextrin derivatives wherein at least two cyclodextrin hydroxy groups are etherified with different groups such as, for example, hydroxy-propyl and hydroxyethyl.

An interesting way of formulating the present compounds in combination with a cyclodextrin or a derivative thereof has been described in EP-A-721,331. Although the formulations described therein are with antifungal active ingredients, they are equally interesting for formulating the compounds of the present invention. The formulations described therein are particularly suitable for oral administration and comprise an antifungal as active ingredient, a sufficient amount of a cyclodextrin or a derivative thereof as a solubilizer, an aqueous acidic medium as bulk liquid carrier and an alcoholic co-solvent that greatly simplifies the preparation of the composition. Said formulations may also be rendered more palatable by adding pharmaceutically acceptable sweeteners and/or flavours.

Other convenient ways to enhance the solubility of the compounds of the present invention in pharmaceutical compositions are described in WO 94/05263, WO 98/42318, EP-A-499,299 and WO 97/44014, all incorporated herein by reference.

More in particular, the present compounds may be formulated in a pharmaceutical composition comprising a therapeutically effective amount of particles consisting of a solid dispersion comprising (a) a compound of formula (I), and (b) one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

The term “a solid dispersion” also comprises dispersions, which are less homogenous throughout than solid solutions. Such dispersions are not chemically and physically uniform throughout or comprise more than one phase.

The water-soluble polymer in the particles is conveniently a polymer that has an apparent viscosity of 1 to 100 mPa.s when dissolved in a 2% aqueous solution at 20° C. solution.

Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

The particles as defined hereinabove can be prepared by first preparing a solid dispersion of the components, and then optionally grinding or milling that dispersion. Various techniques exist for preparing solid dispersions including melt-extrusion, spray-drying and solution-evaporation, melt-extrusion being preferred.

It may further be convenient to formulate the present compounds in the form of nanoparticles which have a surface modifier adsorbed on the surface thereof in an amount sufficient to maintain an effective average particle size of less than 1000 nm. Useful surface modifiers are believed to include those that physically adhere to the surface of the antiretroviral agent but do not chemically bond to the antiretroviral agent.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the present compounds involves a pharmaceutical composition whereby the present compounds are incorporated in hydrophilic polymers and applying this mixture as a coat film over many small beads, thus yielding a composition with good bioavailability which can conveniently be manufactured and which is suitable for preparing pharmaceutical dosage forms for oral administration.

Said beads comprise (a) a central, rounded or spherical core, (b) a coating film of a hydrophilic polymer and an antiretroviral agent and (c) a seal-coating polymer layer.

Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The route of administration may depend on the condition of the subject, co-medication and the like.

Another aspect of the present invention concerns a kit or container comprising a compound of formula (I) in an amount effective for use as a standard or reagent in a test or assay for determining the ability of a potential pharmaceutical to inhibit HIV reverse transcriptase, HIV growth, or both. This aspect of the invention may find its use in pharmaceutical research programs.

The compounds of the present invention can be used in phenotypic resistance monitoring assays, such as known recombinant assays, in the clinical management of resistance developing diseases such as HIV. A particularly useful resistance monitoring system is a recombinant assay known as the Antivirogram®. The Antivirogram® is a highly automated, high throughput, second generation, recombinant assay that can measure susceptibility, especially viral susceptibility, to the compounds of the present invention. (Hertogs K et al. Antimicrob Agents Chemother, 1998; 42(2):269-276, incorporated by reference).

The compounds of the present invention may comprise chemically reactive moieties capable of forming covalent bonds to localized sites such that said compound have increased tissue retention and half-lives. The term “chemically reactive group” as used herein refers to chemical groups capable of forming a covalent bond. Reactive groups will generally be stable in an aqueous environment and will usually be carboxy, phosphoryl, or convenient acyl group, either as an ester or a mixed anhydride, or an imidate, or a maleimidate thereby capable of forming a covalent bond with functionalities such as an amino group, a hydroxy or a thiol at the target site on for example blood components such as albumine. The compounds of the present invention may be linked to maleimide or derivatives thereof to form conjugates.

In still a further aspect, the present invention provides a method of treating patients who are infected by the HIV virus or at risk of becoming infected by the HIV virus, said method comprising the administration of an effective amount of a combination of a compound of formula (I) or a compound of a subgroup of compounds of formula (I), as specified herein, and another HIV-inhibitor, which can be any of the HIV-inhibitors mentioned herein.

The dose of the present compounds or of the physiologically tolerable salt(s) thereof to be administered depends on the individual case and, as customary, is to be adapted to the conditions of the individual case for an optimum effect. Thus it depends, of course, on the frequency of administration and on the potency and duration of action of the compounds employed in each case for therapy or prophylaxis, but also on the nature and severity of the infection and symptoms, and on the sex, age, weight co-medication and individual responsiveness of the human or animal to be treated and on whether the therapy is acute or prophylactic. Customarily, the daily dose of a compound of formula (I) in the case of administration to a patient approximately 75 kg in weight is 1 mg to 3 g, preferably 3 mg to 1 g, more preferably, 5 mg to 0.5 g. The dose can be administered in the form of an individual dose, or divided into several, e.g. two, three, or four, individual doses.

### EXAMPLES

The following examples illustrate the preparation of the compounds of formula (I) and their intermediates as well as their pharmacological properties. These examples should not be construed as a limitation of the scope of the present invention.

### Example 1

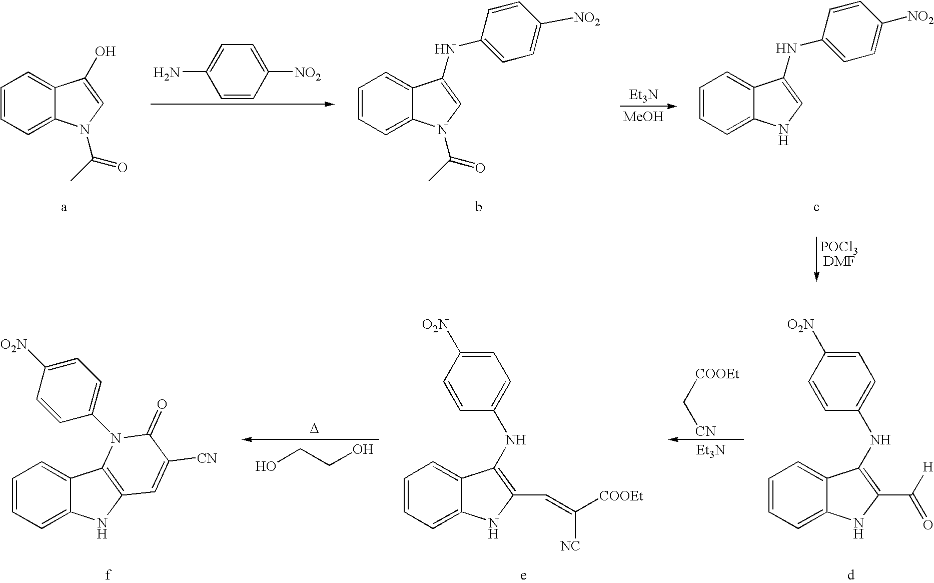

The synthesis of intermediate f started from the commercially available 1-acetyl-1H-indol-3-ol a. Condensation of intermediate a with 4-nitroaniline, under refluxing conditions in acetic acid, yielded 1-acetyl-3-((4-nitrophenyl)amino)indole (b) (Valezheva et al.; Chem. Heterocycl. Compd. (Engl. Transl.); 14; 1978; 757, 759, 760; Khim. Geterotsikl. Soedin.; 14; 1978; 939). Deacylation of intermediate b with triethylamine in refluxing methanol and formylation of intermediate c using phosphorus oxychloride in dimethylformamide resulted in intermediate d (Ryabova, S. Yu.; Tugusheva, N. Z.; Alekseeva, L. M.; Granik, V. G.; Pharm. Chem. J. (Engl. Transl.); EN; 30; 7; 1996; 472-477; Khim. Farm. Zh.; RU; 30; 7; 1996; 42-46). Knoevenagel condensation of intermediate d with ethyl cyanoacetate in the presence of a catalytic amount of triethylamine and subsequent intramolecular cyclisation of intermediate e under reflux in 1,2-ethanediol, yielded intermediate f (2,5-dihydro-1-(4-nitro-phenyl)-2-oxo-1H-pyrido[3,2-b]indole-3-carbonitrile) (Ryabova, S. Yu.; Alekseeva, L. M.; Granik, B. G.; Chem. Heterocycl. Compd. (Engl. Translat.) 36; 3; 2000; 301-306; Khim. Geterotsikl. Soedin.; RU; 3; 2000; 362-367).

More in particular, to a mixture of 1-acetyl-1H-indole-3-ol (a) (0.114 mol, 20 g) in acetic acid (150 ml), was added 4-nitroaniline (1.5 equiv., 0.171 mol, 23.65 g). The mixture was heated at reflux for 5 hours and cooled to room temperature. An orange precipitate was filtered off and washed with isopropanol and diisopropyl ether, affording intermediate b (20.71 g, yield=62%, purity(LC)>98%).

Intermediate b (0.070 mol, 20.71 g) was mixed with methanol (200 ml) and triethylamine (3 equiv., 0.210 mol, 21.27 g) and the mixture was heated at reflux for 4 hours, cooled to room temperature and evaporated under reduced pressure to a dry powder. The crude product c (purity(LC)>95%) was used as such in the next step.

To ice-cooled N,N-dimethylformamide (hereinafter referred to as DMF) (50 ml) was added dropwise phosphorus oxychloride (3 equiv., 0.210 mol, 32.22 g) keeping the internal temperature <10° C. and the cooled mixture was stirred for 1 hour. Then, a solution of c in DMF (100 ml) was added dropwise, keeping the reaction temperature <10° C. during the addition. The ice-bath was removed and the reaction mixture was stirred at room temperature for 1.5 hours. The mixture was poured into ice-water (1 liter) and then heated overnight at 60° C. and cooled to room temperature. The precipitate was isolated by filtration, washed successively with water, isopropanol and diisopropyl ether to afford intermediate d (15.93 g, yield=81%, purity (LC)>95%).

To a mixture of d (0.056 mol, 15.93 g) in isopropanol (150 ml) was added triethylamine (1.5 equiv., 0.085 mol, 8.59 g) and ethyl cyanoacetate (0.068 mol, 7.69 g). The mixture was heated at reflux for 2 hours, cooled to room temperature, filtered and the residue was successively washed with isopropanol and diisopropyl ether to afford intermediate e [S. Yu. Ryabova, L. M. Alekseeva, B. G. Granik Chemistry of Heterocyclic Compounds 2000, 36, 301-306] (16.42 g, yield=78%, purity(LC)>95%).

A stirred suspension of e (0.043 mot 16.42 g) in ethylene glycol (200 ml) was heated at reflux for 2 hours and cooled to room temperature. The precipitate was isolated by filtration and washed successively with isopropanol and diisopropyl ether. Crude intermediate f was crystallized from DMF/water as follows: the crude precipitate was dissolved in warm DMF (250 ml). To the warm solution, water (100 ml) was added and the solution was cooled to room temperature, allowing intermediate f to precipitate. The precipitate was isolated by filtration and washed successively with isopropanol and diisopropyl ether to afford intermediate f (10.52 g, yield=73%, purity (LC)>98%). ¹H NMR (8, DMSO-D6): 6.11 (1H, d, J≈8 Hz), 6.86 (1H, t, J≈8 Hz), 7.38 (1H, t, J≈8 Hz), 7.54 (1H, d, J≈8 Hz), 7.91 (2H, d, J=8.6 Hz), 8.55 (2H, d, J=8.6 Hz), 8.70 (1H, s), 12.00 (1H, br s).

### Example 2

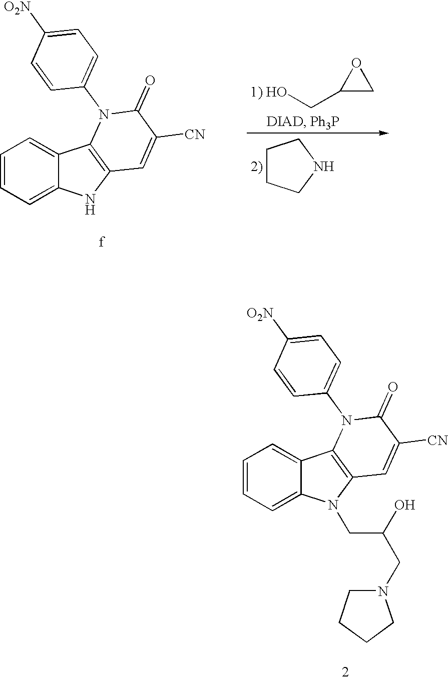

To a cooled (0° C.) solution of intermediate f (0.845 g, 2.56 mmol) in DMF (10 ml) were added glycidol (2 equiv., 5.12 mmol, 0.379 g), triphenylphosphine (2 equiv, 5.12 mmol, 1.342 g) and diisopropyl azodicarboxylate (DIAD) (2 equiv., 5.12 mmol, 1.035 g) and the mixture was stirred overnight at room temperature under N₂-atmosphere. Then, pyrrolidine (20 equiv., 51.16 mmol, 3.64 g) was added and the mixture was heated at 70° C. for 3 hours. The reaction mixture was evaporated under reduced pressure and the dry residue was purified by flash chromatography (silica gel, eluent: 7N NH₃ in methanol/dichloromethane 5/95) affording compound 2 as a yellow powder (1.06 g, yield=91%, purity (LC)>98%). ¹H NMR (DMSO-D6): δ 8.9 (1H, s), 8.55 (2H, d, J≈8 Hz), 7.9 (2H, m), 7.65 (1H, d, J≈9 Hz), 7.4 (1H, t, J≈8 Hz), 6.85 (1H, t, J≈8 Hz), 6.1 (1H, d, J≈8 Hz), 5.1 (1H, s), 4.55 (1H, dd, J_{ab}≈15 Hz, J_{d}≈4 Hz), 4.4 (1H, dd, J_{ab}≈15 Hz, J_{d}≈6 Hz), 4.0 (1H, s), 2.6-2.3 (6H, m), 1.57 (4H, m).

### Example 3

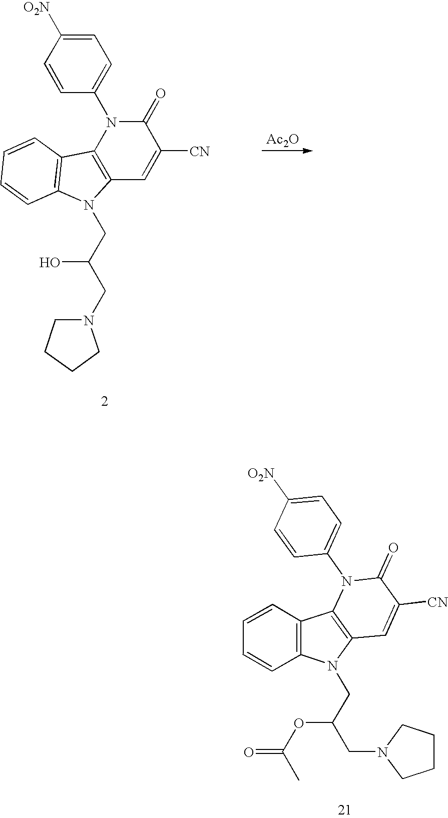

Compound 2 (0.108 g, 0.236 mmol) was stirred at reflux for 2 hours in acetic anhydride (3 ml). Upon cooling, a precipitate was formed. The precipitate was filtered off and washed with isopropanol and diisopropyl ether affording compound 21 (0.103 g, yield=87%, purity (LC)>95%).

### Example 4

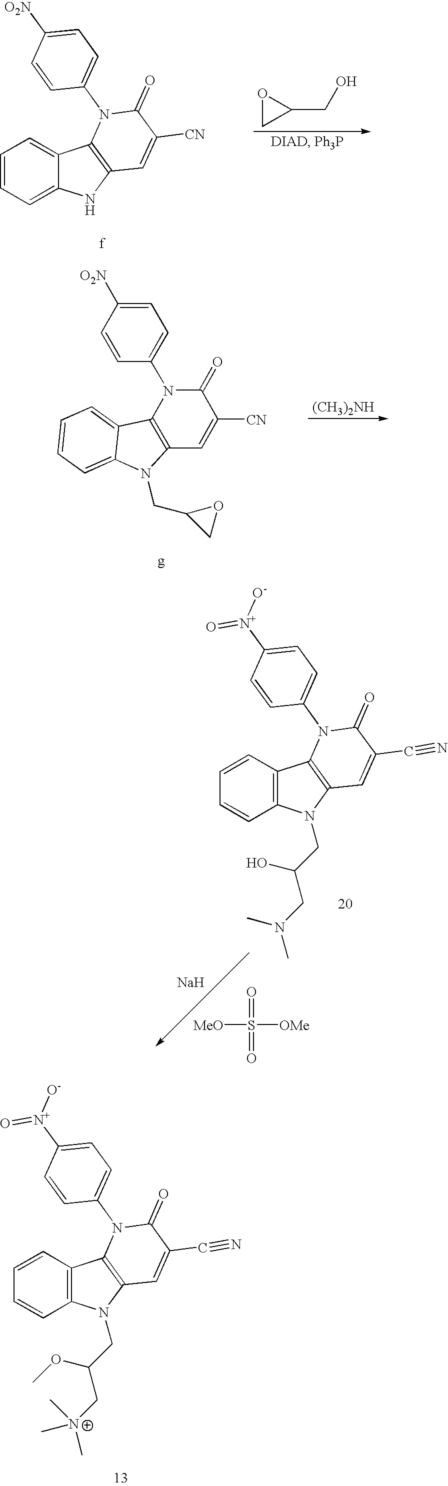

Glycidol (1.5 equiv., 0.673 g, 9.083 mmol), triphenylphosphine (1.5 equiv., 2.382 g, 9.083 mmol) and DIAD (1.5 equiv., 1.837 g, 9.083 mmol) were dissolved in DMP (20 ml) and stirred for 1 hour at 0° C. under nitrogen atmosphere. Intermediate f (2.00 g, 6.055 mmol) was added and the reaction mixture was stirred overnight at room temperature. Water was added causing precipitation of the crude intermediate g. The precipitate was isolated by filtration, washed with water and dissolved in ethanol (20 ml). The mixture was heated at 50° C. and cooled to room temperature. The precipitate was filtered off and washed with ethanol and diisopropyl ether to afford epoxide g (1.867 g, yield=74.2%, purity (LC)=93%)

To a stirred solution of g (0.200 g, 0.414 mmol) in DMF (3 ml) was added a solution of dimethylamine 40% in water (10 equiv., 4.14 mmol, 0.524 ml). The mixture was heated overnight at 65° C. and allowed to cool to room temperature, allowing the reaction product to precipitate from the reaction mixture. The product was filtered off, washed with water, isopropanol and diisopropyl ether. Recrystallisation from DMF afforded compound 20 (0.100 g, yield=56%, purity (LC)>95%).

To a stirred solution of compound 20 (50 mg, 0.120 mmol) in DMF (3 ml) was added sodium hydride (1.2 equiv., 0.144 mmol, 6 mg of a suspension of 60% NaH in mineral oil) and dimethyl sulfate (10 equiv., 1.20 mmol, 0.150 g) and the reaction mixture was stirred for 1 hour at room temperature under nitrogen atmosphere. Water was added and the aqueous layer washed with ethyl acetate. The water layer was concentrated under reduced pressure and the residue was recrystallized from a water/methanol mixture. The crystals were isolated by filtration, washed with isopropanol and diisopropyl ether affording compound 13 (0.036 g, yield=60%, Purity=89%).

### Example 5

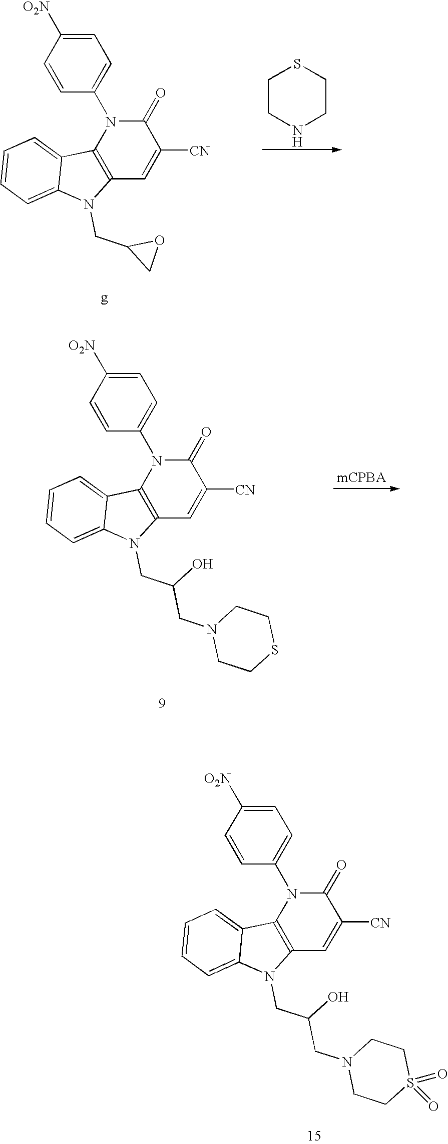

To a stirred solution of compound g (0.400 g, 1.04 mmol) in DMF (5 ml) was added thiomorpholine (5 equiv., 5.18 mmol, 0.534 g). The mixture was heated overnight at 65° C. and allowed to cool at room temperature. The mixture was filtered, washed with water, isopropanol and diisopropyl ether. The solid was recrystallized from DMF, filtered off and washed with isopropanol and diisopropyl ether to afford compound 9 (0.352 g, yield=66.7%, purity (LC)>96%).

To a stirred mixture of compound 9 (0.227 g, 0.464 mmol) in dichloromethane (4 ml) was added 3-chloroperbenzoic acid (2.2 equiv., 0.176 g, 1.02 mmol). The reaction mixture was stirred at room temperature for 20 min. During this period, the reaction product precipitated from the solution. The crystals were isolated by filtration and washed with dichloromethane and diisopropyl ether to afford compound 15 (0.208 g, yield=80%, Purity (LC)=93%).

### Example 6

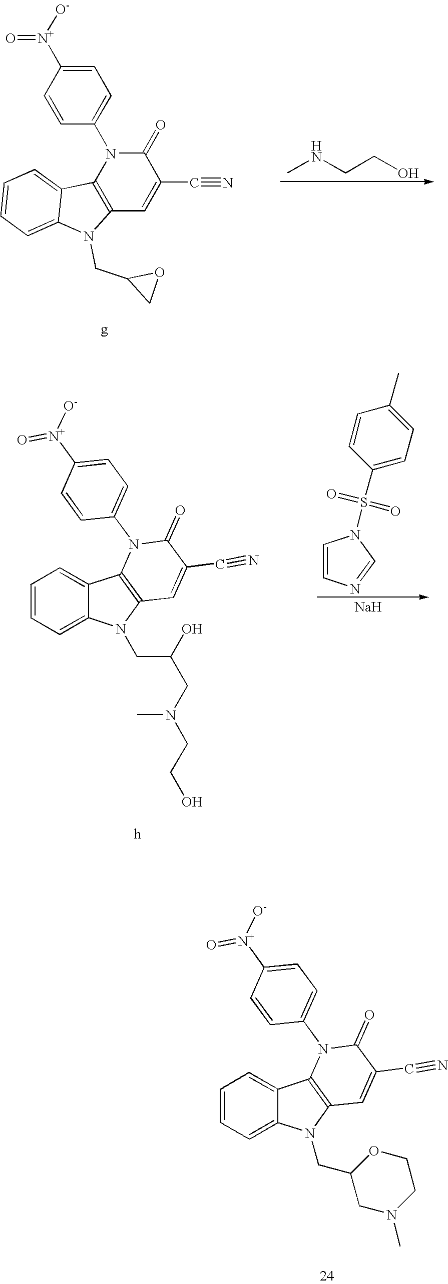

Compound g (0.300 g, 0.621 mmol) was dissolved in DMP (3 ml). 2-Methylamino-ethanol (10 equiv., 6.21 mmol, 0.467 g) was added and the reaction mixture was heated at 65° C. overnight. Upon cooling to room temperature, a solid precipitated from the reaction mixture and it was isolated by filtration and washed with water, isopropanol and diisopropyl ether. The solid was recrystallized from DMF, filtered off and washed with isopropanol and diisopropyl ether to afford compound h (0.193 g, yield=56%, purity (LC)>83%).

In a flask, provided with a CaCl₂-drying tube, compound h (0.193 g, 0.418 mmol) was dissolved in THF (4 ml) and cooled to 0° C. Sodium hydride (2.5 equiv., 1.05 mmol, 42 mg of a suspension of 60% NaH in mineral oil) was added in one portion and the mixture was stirred at 0° C. for 20 min. p-Toluensulfonyl imidazole (1.1 equiv., 0.102 g, 0.460 mmol) was added and the reaction mixture was stirred overnight at room temperature. Evaporation under reduced pressure and purification of the crude reaction mixture by reversed phase HPLC, afforded compound 24 (6 mg, yield=3%, purity (LC)>950%).

### Example 7

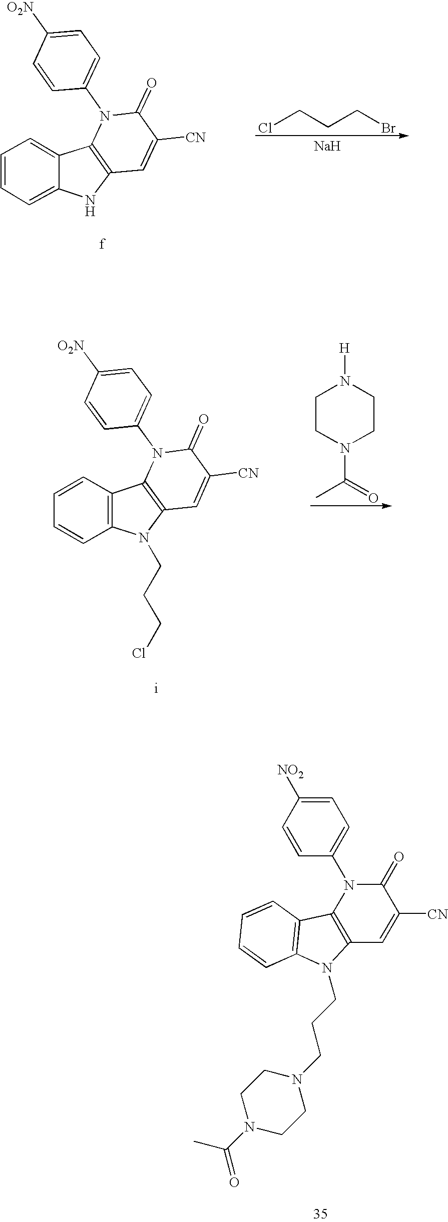

Compound f (2.0 g, 6.055 mmol) was dissolved in DMF (25 ml). Sodium hydride (1.2 equiv., 0.290 g of a suspension of 60% NaH in mineral oil, 7.266 mmol) was added and the reaction mixture was heated at 100° C. for 1 hour and allowed to cool to room temperature. 1-Bromo-3-chloro-propane (1.5 equiv., 1.430 g, 9.083 mmol) was added and the mixture was stirred at room temperature for 3 hours. The reaction product i precipitated upon the addition of water. The solid was isolated by filtration and washed with water, isopropanol and diisopropyl ether affording intermediate i (2.334 g, yield=95%, purity=(LC)>95%) as a dark orange powder.

Compound i (0.150 g, 0.369 mmol) and 1-acetylpiperazine (3 equiv., 1.11 mmol, 0.142 g) were mixed in DMF (3 ml). The mixture was heated at 70° C. for 5 hours. A second portion of 1-acetylpiperazine (3 equiv., 1.11 mmol, 0.142 g) was added and the mixture was heated at 70° C. overnight. The reaction mixture was cooled to room temperature, precipitated with water, filtered off and successively washed with isopropanol and diisopropyl ether. Purification by flash chromatography on silica gel (eluent dichloromethane/methanol: 9/1) gave compound 35 (0.122 g, yield=63%, purity (LC)=94%).

### Example 8

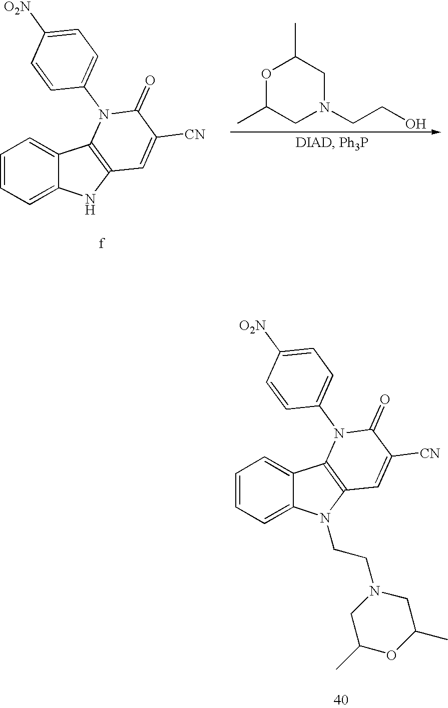

2-(2,6-Dimethyl-morpholin-4-yl)ethanol (2 equiv., 0.145 g, 0.908 mmol), triphenylphosphine (2 equiv., 0.238 g, 0.908 mmol) and DIAD (2 equiv., 0.184 g, 0.908 mmol) were mixed in DMF (4 ml) and stirred at 0° C. for 15 min. Compound f (0.150 g, 0.454 mmol) was added and the mixture was stirred overnight at room temperature. Water was added and the precipitate was isolated by filtration. The precipitate was mixed with ethanol and heated to 50° C. After cooling to room temperature, the precipitate was filtered off and washed with ethanol and diisopropyl ether to give compound 40 (0.170 g, yield=79.4%, purity (LC)>95%).

### Example 9

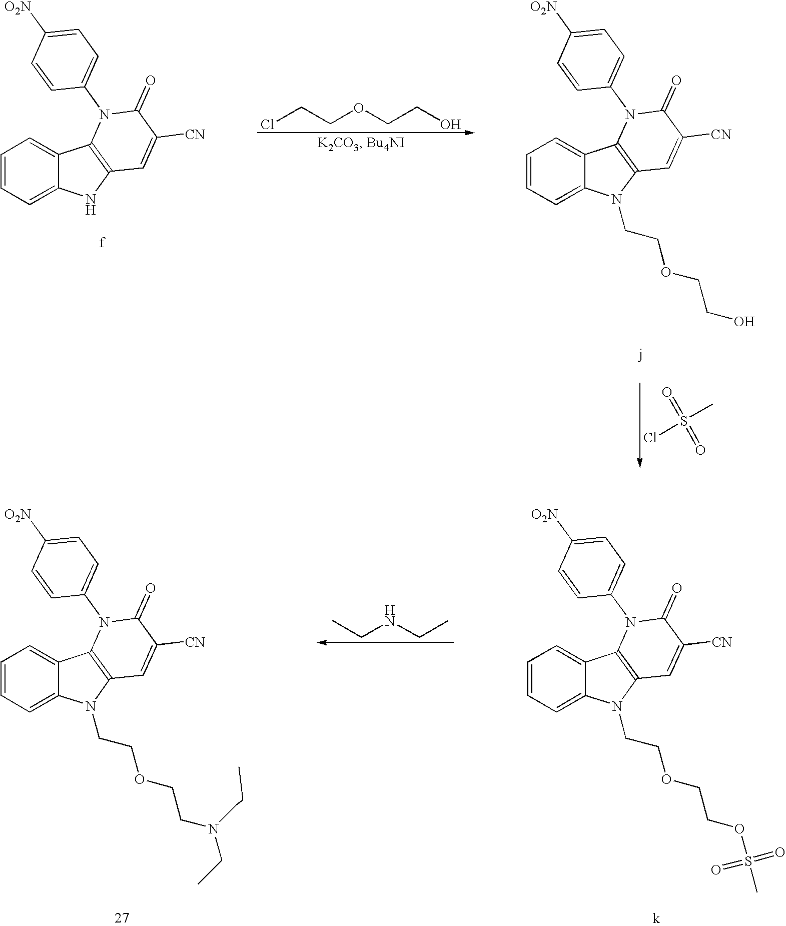

A mixture of f (0.500 g, 1.51 mmol), potassium carbonate (1.256 g, 9.06 mmol, 6 equiv.), 2-(2-chloro-ethoxy)-ethanol (1.128 g, 9.06 mmol, 6 equiv.) and tetrabutyl-ammonium iodide (1.673 g, 3.51 mmol, 3 equiv.) in DMF (20 ml) was heated under nitrogen at 60° C. for 10 hours. Water was added to the warm solution, and the precipitate was filtered off and washed with isopropanol and diisopropylether, affording compound j (0.460 g, yield=58.1%, purity=83%).

A mixture of compound j (0.460 g, 1.10 mmol), pyridine (0.434 g, 5.50 mmol, 5 equiv.) and methanesulfonyl chloride (0.377 g, 3.30 mmol, 3 equiv.) in dichloromethane (10 ml), was stirred at room temperature for 24 hours. The reaction mixture was diluted with dichloromethane until a clear solution was obtained, and this solution washed with a 1N hydrochloric acid solution and a saturated aqueous NaHCO₃ solution. The organic phase was evaporated under reduced pressure to give crude intermediate k (purity=83%) and was used as such in next step.

To a solution of crude compound k (0.181 g, 0.37 mmol) in DMF (15 ml) was added diethylamine (0.266 g, 3.7 mmol, 10 equiv.) and the mixture was heated for 8 hours at 60° C. Water was added to the mixture causing the reaction product to precipitate. The precipitate was isolated by filtration and washed with isopropanol and diisopropylether. The product was further purified by chromatography on silica gel using dichloro methane/methanol (90/10) as the eluent to give compound 27 (0.030 g, yield=17% (2 steps), purity=99.5%).

### Example 10

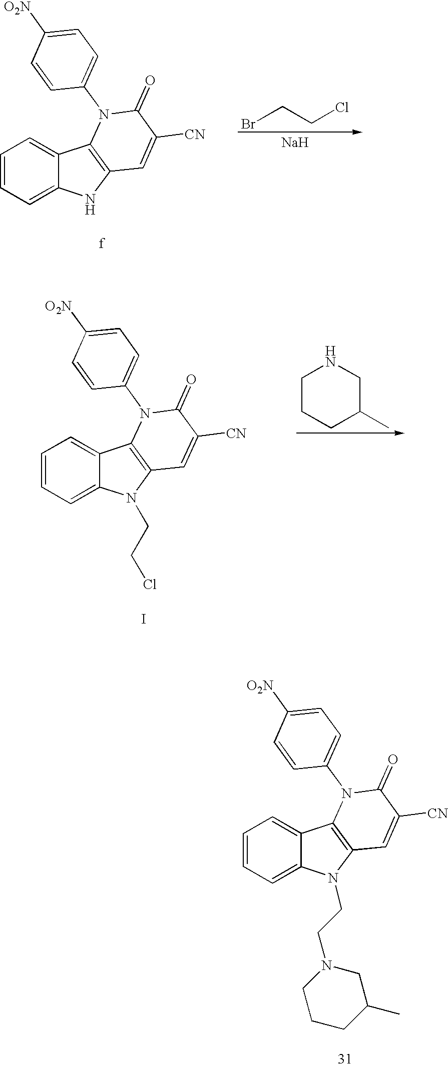

To a mixture of compound f (6 mmol 2.00 g) in DMF (50 ml), was added sodium hydride (2 equiv., 12.1 mmol, 484 mg of 60% NaH in mineral oil) and the mixture was heated for 1 hour to 50° C. The mixture was cooled to room temperature and 1-bromo-3-chloroethane (5 equiv., 15 mmol, 4.343 g) was added. The reaction mixture was stirred overnight at room temperature. The reaction mixture containing compound 1 (purity=85%) was used as such in the next step.

3-Methylpiperidine (1.5 equiv., 0.76 mmol, 0.076 g) was added to 5 ml of the crude reaction mixture of compound 1 (0.51 mmol) and the mixture was heated for 5 hours at 70° C. The solvent was removed under reduced pressure and the reaction product was purified by preparative reversed phase HPLC to afford compound 31 (0.025 g, yield=9.7%, purity (LC)>90%).

### Example 11

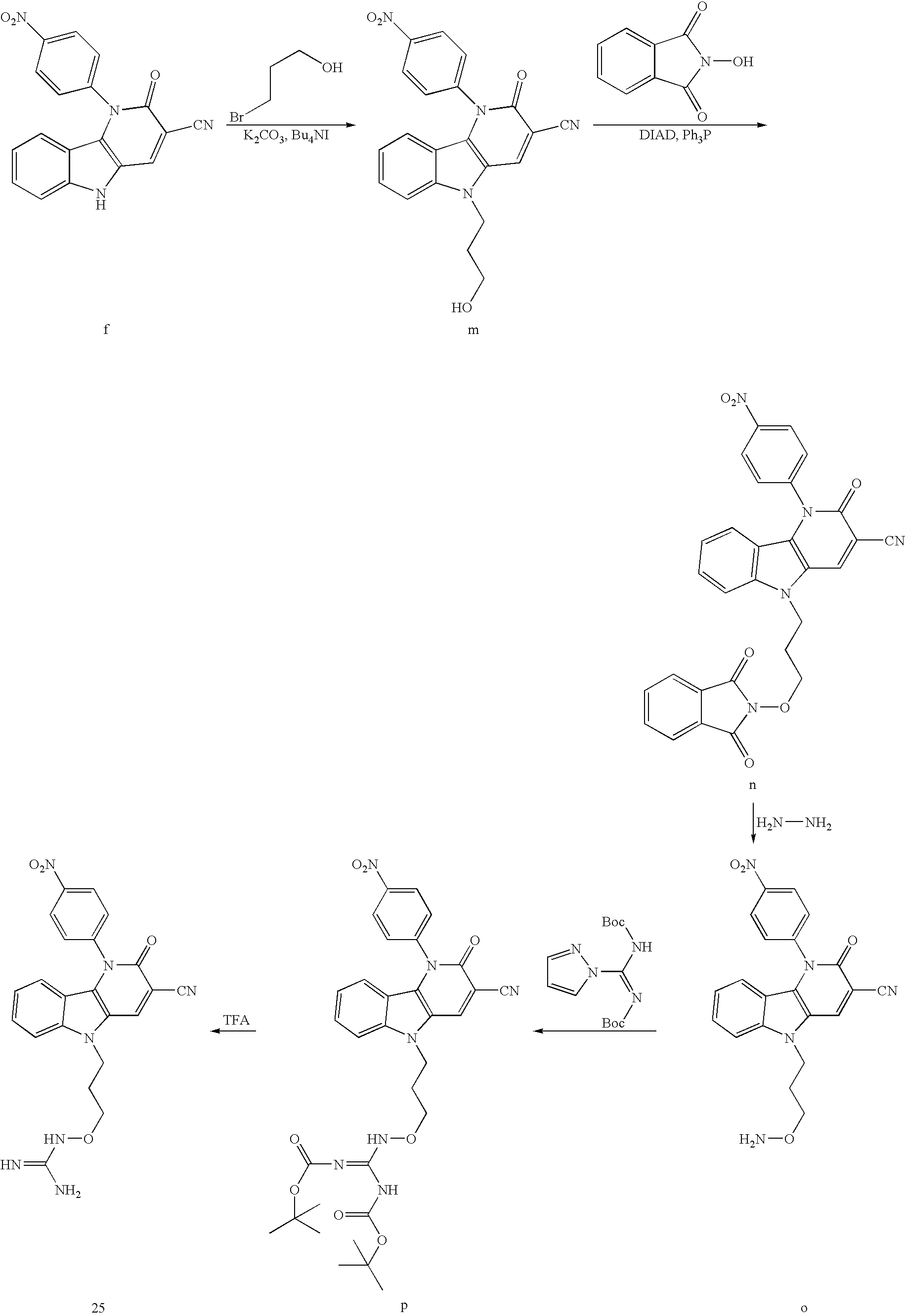

To a mixture of f (6.06 mmol, 2.00 g) in dry DMF (20 ml) under N₂-atmosphere, was added 3-bromo-1-propanol (2.5 equiv., 15.1 mmol, 2.10 g), tetrabutylammonium iodide (1 equiv., 6.06 mmol, 2.24 g) and potassium carbonate (2.5 equiv., 15.1 mmol, 2.09 g). The mixture was stirred at room temperature for 48 hours. The reaction mixture was evaporated under reduced pressure to a dry residue. The residue was mixed with water, extracted with dichloromethane and the combined organic fractions were dried (MgSO₄) and evaporated under reduced pressure to a dry powder. The powder was washed with ethanol and diisopropylether to afford intermediate m (2.30 g, yield 97.8%, purity (LC)=90.7%).

A mixture of intermediate m (6.0 mmol, 2.30 g), N-hydroxyphthalimide (2.00 equiv., 12.1 mmol, 1.97 g) and triphenylphosphine (2.00 equiv., 12.1 mmol, 3.17 g) in dry DMF (15 ml) was cooled to 0° C. At this temperature, diisopropylazodicarboxylate (2.00 equiv., 12.1 mmol, 2.45 g) was added dropwise and the reaction mixture was stirred overnight at room temperature. The reaction mixture was evaporated under reduced pressure to a dry powder and the residue was mixed with water. The product was extracted with dichloromethane and dried over MgSO₄. After filtration and evaporation under reduced pressure the powder washed with methanol and dried under vacuum at 50° C. to afford compound n (2.31 g, yield=71.6%, purity (LC)=97%).

To a mixture of n (0.69 mmol, 0.37 g) in methanol (10 ml) was added hydrazine monohydrate (10 equiv., 6.9 mmol, 0.345 g). The mixture was heated at reflux for 3 minutes and was evaporated under reduced pressure to a dry powder. Water was added, the product was extracted with dichloromethane and dried over MgSO₄. Filtration and evaporation under reduced pressure afforded compound o (270 mg, yield=97%, purity (LC)=91.5%).

To a mixture of o (0.34 mmol, 135 mg) in DMF (3 ml) under N₂-atmosphere, was added (tert-butoxycarbonylimino-pyrazol-1-yl-methyl)-carbamic acid tert-butyl ester (1.2 equiv., 0.402 mmol, 125 mg). The mixture was stirred at room temperature for 10 hours. The reaction mixture was mixed with water and the precipitate was filtered off. The product was purified using column chromatography (eluent: methanol/dichloro methane 2:98) to afford compound p (147 mg, yield=68.0%, purity (LC)=96%).

To a mixture of p (0.12 mmol, 75 mg) in dichloromethane (25 ml) was added trifluoroacetic acid (1 ml). The mixture was stirred at room temperature for 10 hours and the solvent was evaporated under reduced pressure. The residue was crystallized from ethanol to afford compound 25 (13 mg, yield=25%, purity (LC)=93%).

### Example 12

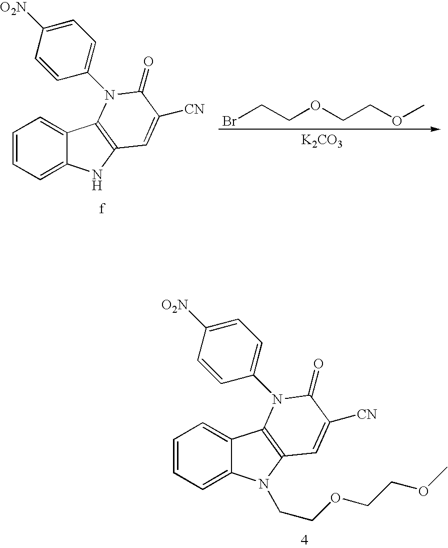

A mixture of intermediate f (105 mg, 0.318 mmol), 1-bromo-2-(2-methoxyethoxy)-ethane (76 mg, 0.41 mmol), and K₂CO₃ (57 mg, 0.41 mmol) in DMF (5 ml) was stirred at room temperature for 48 hours. The reaction mixture was partitioned between water (20 ml) and ethyl acetate (30 ml), dried (Na₂SO₄) and evaporated. The residue was triturated in diethyl ether (3 ml), and filtered off. The yellow prisms were washed with diethyl ether and hexane to give the target product 4 (51 mg, yield=37%).

### Example 13

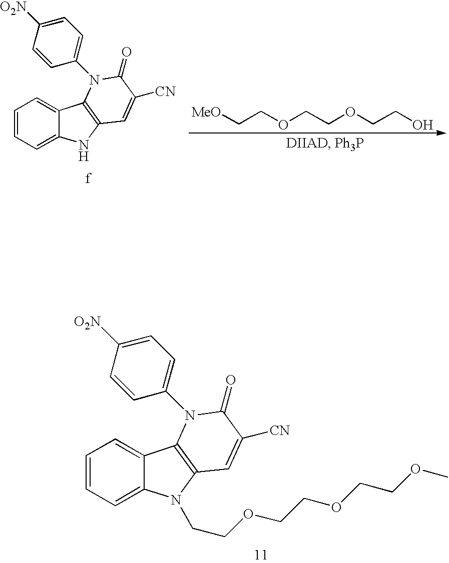

DIAD (0.245 g, 1.21 mmol) was added under N₂ to a solution of intermediate f (200 mg, 0.606 mmol), triphenylphosphine (318 mg, 1.21 mmol) and 2-[2-(2-methoxyethoxy)ethoxy]ethanol (240 μl, 1.21 mmol), in dry DMF (15 ml). After 2 hours, the reaction mixture was partitioned between water and ethyl acetate, dried (Na₂SO₄), and evaporated. Purification of the crude material by column chromatography on silica gel (eluent: 100% THF) afforded the target product 11 (89 mg, yield=31%) as a yellow powder.

### Example 14

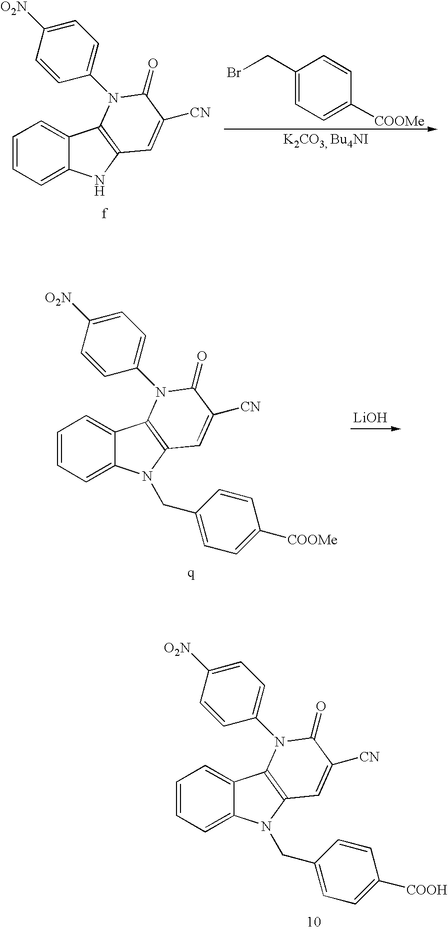

A mixture of compound f (200 mg, 0.606 mmol), K₂CO₃ (126 mg, 0.908 mmol), tetrabutylammonium iodide (300 mg, 0.812 mmol) and methyl 4-(bromomethyl)-benzoate (250 mg, 11.09 mmol) in THF (15 ml) was stirred at 65° C. for 12 hours. Then, the solvent was evaporated and the residue partitioned between ethyl acetate and water, dried (Na₂SO₄) and evaporated. The residue was triturated in diethyl ether and filtered off to give the target product q (260 mg, yield=89%, purity (LC)>98%) as a yellow powder.

A solution of methyl 4-[[3-cyano-1-(4-nitrophenyl)-2-oxo-2,5-dihydro-1H-pyrido-[3,2-b]indol-5-yl]methyl]benzoate (q) (260 mg, 0.543 mmol) and LiOH (170 mg, 7.06 mmol) in (MeOH/THF/H₂O, 5:4:1, 30 ml) was stirred at room temperature for 72 hours. The reaction mixture was partitioned between water and ethyl acetate, the pH of the water layer was adjusted at 2 with concentrated hydrochloric acid and extracted with ethyl acetate, dried (Na₂SO₄) and evaporated. Purification by flash chromatography (silica gel, eluent: 100% THF) afforded the target product 10 (20 mg, yield=7.9%) as a yellow powder.

### Example 15

Synthesis of Compounds with X is O

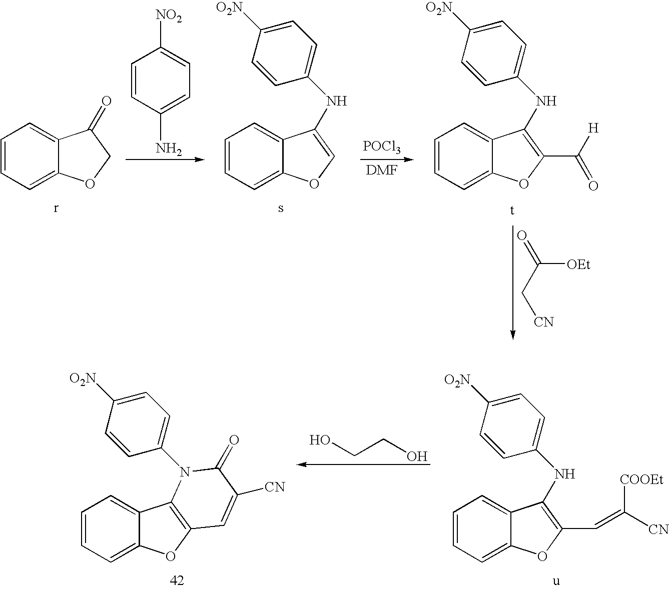

To a mixture of 3-hydroxybenzofuran r (0.0373 mol, 5 g) in toluene (100 ml), was added 4-nitroaniline (1 equiv., 0.0373 mol, 5.149 g) and a catalytic amount of p-toluenesulfonic acid. The mixture was heated to reflux for 2 hours and cooled to room temperature. The precipitate was filtered off and washed with isopropanol and diisopropyl ether, affording intermediate s [V. A. Azimov, S. Yu. Ryabova, L. M. Alekseeva and V. G. Granik Chemistry of heterocyclic compounds 2000, 36, 1272-1275] (6.28 g, yield=66%, purity (LC)>95%).

To ice-cooled DMF (20 ml) was added dropwise phosphorus oxychloride (3 equiv., 0.074 mol, 11.36 g) keeping the internal temperature <10° C. Then, a solution of intermediate s (0.024 mol, 6.10 g) in DMF (50 ml) was added dropwise, keeping the reaction temperature <10° C. during the addition. The reaction mixture was stirred at 0° C. for 2 hours. The mixture was poured into ice-water (250 ml), heated for 2 hours at 60° C. and then cooled to room temperature. The precipitate was isolated by filtration, washed successively with water, isopropanol and diisopropyl ether to afford intermediate t [V. A. Azimov, S. Yu. Ryabova, L. M. Alekseeva and V. G. Granik Chemistry of heterocyclic compounds 2000, 36, 1272-1275] (5.98 g, yield=86%, purity (LC)=95%).

To a stirred mixture of intermediate t (7.036 mmol, 2.00 g) in isopropanol (25 ml) was added triethylamine (1.5 equiv., 10.55 mmol, 1.068 g) and ethyl cyanoacetate (1.2 equiv., 8.44 mmol, 0.955 g). The mixture was heated at reflux for 4 hours, cooled to room temperature, filtered off and the precipitate was successively washed with isopropanol and diisopropyl ether to afford intermediate u (2.00 g, yield=75%, purity (LC)>95%).

A stirred suspension of intermediate u (5.30 mmol, 2.00 g) in ethyleneglycol (30 ml) was heated at reflux for 1 hour and cooled to room temperature. The precipitate was isolated by filtration and washed successively with isopropanol and diisopropyl ether. The product was crystallised from DMF/water. The precipitate was isolated by filtration and washed successively with isopropanol and diisopropyl ether to afford compound 42 (1.065 g, yield=61%, purity (LC)>98%). ¹H NMR (DMSO-D6): δ 9.05 (s, 1H), 8.57 (d, J 8.7 Hz, 2H), 7.97 (d, J 8.7 Hz, 2H), 7.83 (d, J≈8.5 Hz, 1H), 7.62 (t, J≈7.8 Hz, 1H), 7.19 (t, J≈7.7 Hz, 1H), 6.30 (d, J≈8.1 Hz, 1H)

The following tables list examples of compounds of the present invention which compounds are prepared analogous those of the foregoing synthesis schemes.

In the following tables the column headed “rf” lists the retention times and the column “(M+H)⁺” lists the mass of the molecule ions.

Retention times were measured using the following equipment: HPLC-system:

Waters Alliance 2790 (pump+auto sampler), Waters 996 (Photo diode array-detector);

Column: Waters XTerra MS C18 2.5 μm 50×4.6 mm. The following were the measurement parameters:

Temperature: 30° C.

Mobile phase:

A: 10 mM HCOONH4+0.1% HCOOH in H₂O
B: 0.1% HCOOH in CH₃CN
   Gradient: 0 min: 15% B, 5 min: 95% B, 7 min: 95% B
   Equilibration time: 2 min
   Flow: 1.2 ml/min
   Injection volume: 3 ul of a 1 mg/ml solution

The molecular ion was determined using the following MS-detector: Waters LCT; ionisation: electrospray in positive or negative mode.

TABLE 1

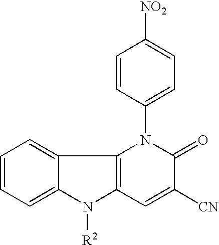

| Comp. | Synthesis | | | | |
|---|---|---|---|---|---|
| No. | Example | R² | Salt | rf | (M + H)⁺ |
| 1 | 2 | 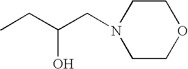 | | 2.29 | 474 |
| 2 | 2 | 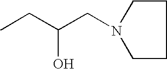 | | 2.39 | 458 |
| 3 | 2 | 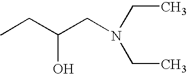 | | 2.44 | 460 |
| 4 | 12 | —[(CH₂)₂—O]₂—CH₃ | | 3.61 | 433 |
| 5 | 8 | 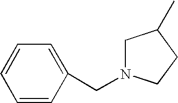 | | | |
| 6 | 2 | 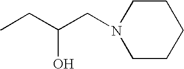 | | | |
| 7 | 2 | 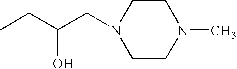 | | 2.35 | 487 |
| 8 | 2 | 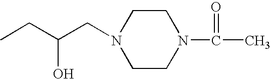 | | 2.25 | 515 |
| 9 | 5 | 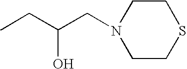 | | 2.43 | 490 |
| 10 | 14 | 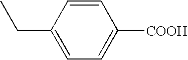 | | 3.49 | 465 |
| 11 | 13 | —[(CH₂)₂—O]₃—CH₃ | | 3.53 | 477 |
| 12 | 13 | —[(CH₂)₂—O]₄—CH₃ | | 3.49 | 521 |
| 13 | 4 | 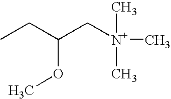 | sulfate | 2.53 | 461 |
| 14 | 2 | 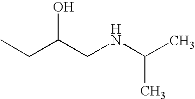 | | 2.38 | 487 |
| 15 | 5 | 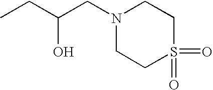 | | 2.28 | 522 |
| 16 | 2 | 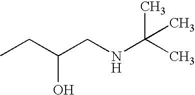 | | 2.54 | 460 |
| 17 | 2 | 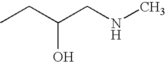 | | 2.25 | 418 |
| 18 | 2 | 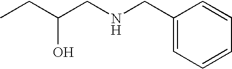 | | 2.68 | 494 |
| 19 | 2 | 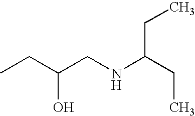 | | 2.7 | 474 |
| 20 | 4 | 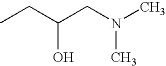 | | | |
| 21 | 3 | 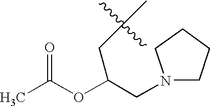 | | 2.51 | 500 |
| 22 | 6 | 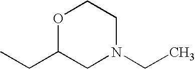 | | 2.51 | 458 |
| 23 | 8 | 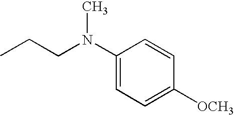 | | | |
| 24 | 6 | 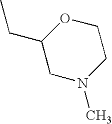 | | 2.31 | 444 |
| 25 | 11 | 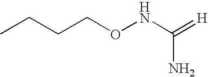 | trifluoro acetate | 2.53 | 446 |
| 26 | 9 | 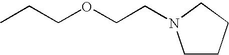 | | 2.53 | 472 |
| 27 | 9 | 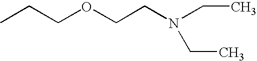 | | 2.64 | 474 |
| 28 | 9 | 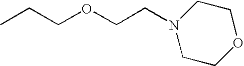 | | 2.49 | 488 |
| 29 | 7 | 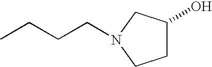 | | 2.36 | 458 |
| 30 | 7 | 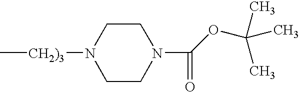 | | 3.05 | 557 |
| 31 | 10 | 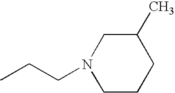 | | 2.69 | 456 |
| 32 | 7 | 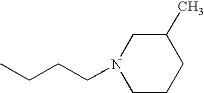 | | 2.76 | 470 |
| 33 | 7 | 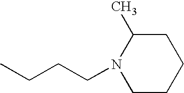 | | 2.66 | 470 |
| 34 | 7 | 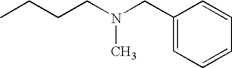 | | 2.84 | 492 |
| 35 | 7 | 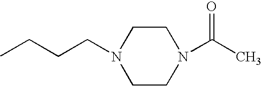 | | 2.38 | 499 |
| 36 | 7 | 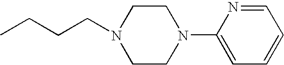 | | 2.74 | 534 |
| 37 | 7 | 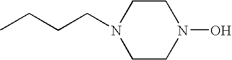 | | 2.36 | 472 |
| 38 | 7 | 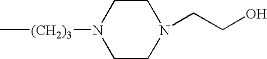 | | 2.46 | 501 |
| 39 | 7 | 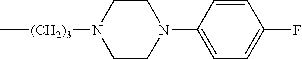 | | 3.14 | 551 |
| 40 | 8 | 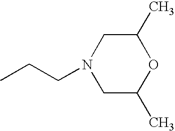 | | 2.98 | 472 |
| 41 | 8 | 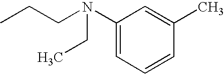 | | | |

TABLE 2

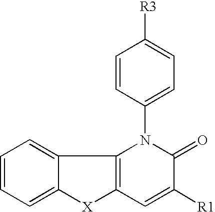

| | Synthesis | | | | | |
|---|---|---|---|---|---|---|
| Comp. No. | Example | X | R¹ | R³ | rf | (M + H)⁺ |
| 42 | 15 | O | CN | NO₂ | 3.61 | 332 |

### Example 16

### In Vitro Inhibition of HIV Reverse Transcriptase

The assay was run using kit TRK 1022 (Amersham Life Sciences) according to the manufacturer's instructions with slight modifications. Test compounds were diluted in steps of 1/4 in 100% DMSO and subsequently transferred to Medium A (1/50 dilution; medium A: RPMI 1640+10% FetalClone 1+Gentamycin 20 mg/L). 25 μl of compound (in 2% DMSO in Medium A) or 25 μl of 2% DMSO in medium A was added to wells. To each well was added 25.5 μl master mix (master mix: 5 μl primer/template beads, 10 μl assay buffer, 0.5 μl tracer (3H-TTP), 5 μl HIV RT enzyme solution at a final enzyme activity of 15 mU per 50 μl reaction, 5 μl medium A). The plates were sealed, marked as radioactive and incubated during 4 hours at 37° C. Subsequently, 100 μl stop solution was added to each well (except R1). The radioactivity was counted in a TopCount.

Compounds 1, 2 and 9 inhibit IV reverse transcriptase in vitro and consequently do not need conversion to an active metabolite in order to inhibit reverse transcriptase.

### Example 17

### Cellular Assay

Compounds of the present invention were examined for anti-viral activity in a cellular assay, which was performed according to the following procedure.

HIV- or mock-infected MT4 cells were incubated for five days in the presence of various concentrations of the inhibitor. At the end of the incubation period, the replicating virus in the control cultures has killed all HIV-infected cells in the absence of any inhibitor. Cell viability was determined by measuring the concentration of MTT, a yellow, water soluble tetrazolium dye that is converted to a purple, water insoluble formazan in the mitochondria of living cells only. Upon solubilization of the resulting formazan crystals with isopropanol, the absorbance of the solution was monitored at 540 nm. The values correlate directly to the number of living cells remaining in the culture at the completion of the five day incubation. The inhibitory activity of the compound was monitored on the virus-infected cells and was expressed as EC₅₀ and EC₉₀. These values represent the amount of the compound required to protect 50% and 90%, respectively, of the cells from the cytopathogenic effect of the virus. The toxicity of the compound was measured on the mock-infected cells and was expressed as CC₅₀, which represents the concentration of compound required to inhibit the growth of the cells by 50%. The selectivity index (SI) (ratio CC₅₀/EC₅₀) is an indication of the selectivity of the anti-HIV activity of the inhibitor. Wherever results are reported as e.g. pEC₅₀ or pCC₅₀ values, the result is expressed as the negative logarithm of the result expressed as EC₅₀ or CC₅₀ respectively.

The following table 3 lists the pEC₅₀ values obtained in this test for a number of compounds of this invention.

TABLE 3

| Comp. No. | pEC₅₀ |
|---|---|
| 1 | 5.5 |
| 2 | 6.6 |
| 3 | 6.7 |
| 4 | 6.5 |
| 7 | 6.0 |
| 8 | 4.9 |
| 9 | 6.1 |
| 11 | 5.8 |
| 12 | 5.3 |
| 13 | 4.8 |
| 14 | 5.9 |
| 16 | 5.8 |
| 17 | 5.8 |
| 18 | 6.1 |
| 19 | 6.7 |
| 21 | 6.3 |
| 22 | 5.3 |
| 23 | 6.0 |
| 25 | 4.8 |
| 26 | 5.9 |
| 27 | 5.9 |
| 28 | 5.8 |
| 29 | 5.8 |
| 30 | 5.7 |
| 31 | 6.0 |
| 32 | 6.7 |
| 33 | 6.9 |
| 34 | 6.1 |
| 35 | 5.6 |
| 36 | 5.3 |
| 37 | 6.0 |
| 38 | 5.6 |
| 39 | 5.9 |
| 40 | 6.0 |
| 41 | 5.4 |
| 42 | 6.1 |

### Example 18

### Formulations

Capsules

Active ingredient, in casu a compound of formula (I), is dissolved in organic solvent such as ethanol, methanol or methylene chloride, preferably, a mixture of ethanol and methylene chloride. Polymers such as polyvinylpyrrolidone copolymer with vinyl acetate (PVP-VA) or hydroxypropylmethylcellulose (HPMC), typically 5 mPa.s, are dissolved in organic solvents such as ethanol, methanol methylene chloride. Suitably the polymer is dissolved in ethanol. The polymer and compound solutions are mixed and subsequently spray dried. The ratio of compound/polymer is selected from 1/1 to 1/6. Intermediate ranges can be 111.5 and 1/3. A suitable ratio can be 1/6. The spray-dried powder, a solid dispersion, is subsequently filled in capsules for administration. The drug load in one capsule ranges between 50 and 100 mg depending on the capsule size used.

Film-Coated Tablets

Preparation of Tablet Core

A mixture of 100 g of a compound of formula (I), 570 g lactose and 200 g starch are mixed well and thereafter humidified with a solution of 5 g sodium dodecyl sulfate and 10 g polyvinylpyrrolidone in about 200 ml of water. The wet powder mixture is sieved, dried and sieved again. Then there is added 100 g microcrystalline cellulose and 15 g hydrogenated vegetable oil. The whole is mixed well and compressed into tablets, giving 10.000 tablets, each comprising 10 mg of the active ingredient.

Coating

To a solution of 10 g methylcellulose in 75 ml of denaturated ethanol there is added a solution of 5 g of ethylcellulose in 150 ml of dichloromethane. Then there is added 75 ml of dichloromethane and 2.5 ml 1,2,3-propanetriol. 10 g of polyethylene glycol is molten and dissolved in 75 ml of dichloromethane. The latter solution is added to the former and then there is added 2.5 g of magnesium octadecanoate, 5 g of polyvinylpyrrolidone and 30 ml of concentrated color suspension and the whole is homogenated. The tablet cores are coated with the thus obtained mixture in a coating apparatus.

1. A compound of formula (I), 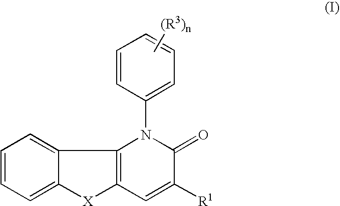
an N-oxide, salt, stereoisomeric form, racemic mixture, prodrug, ester or metabolite thereof, wherein
X is a bivalent radical NR², O, S, SO, SO₂;
R¹ is hydrogen, cyano, halo, aminocarbonyl, hydroxycarbonyl, C₁₋₄alkyloxycarbonyl, C₁₋₄alkylcarbonyl, mono- or di(C₁₋₄alkyl)aminocarbonyl, arylaminocarbonyl, N-(aryl)-N—(C₁₋₄alkyl)aminocarbonyl, methanimidamidyl, N-hydroxy-methanimidamidyl, mono- or di(C₁₋₄alkyl)methanimidamidyl, Het₁ or Het₂;
n is 1, 2 or 3;
R² is:
i) aryl substituted with a radical —COOR⁴; or R² is
ii) C₁₋₁₀alkyl, C₂₋₁₀alkenyl, C₃₋₇cycloalkyl,
each of said C₁₋₁₀alkyl, C₂₋₁₀alkenyl, C₃₋₇cycloalkyl, each individually and independently, being substituted with aryl wherein said aryl is substituted with a radical —COOR⁴; or R² is
iii) C₁₋₁₀-alkyl, C₂₋₁₀alkenyl, C₃₋₇cycloalkyl, each individually and independently, substituted with a radical selected from —NR^{5a}—C(═NR^{5b})—NR^{5c}R^{5d}, —NR^{5a}—C(═NR^{5e})—R^{5f}, —O—NR^{5a}—C(═NR^{5b})—NR^{5c}R^{5d}, —O—NR^{5a}—C(═NR^{5e})—R^{5f}, -sulfonyl-R⁶, —NR⁷R⁸, —NR⁹R¹⁰, a radical 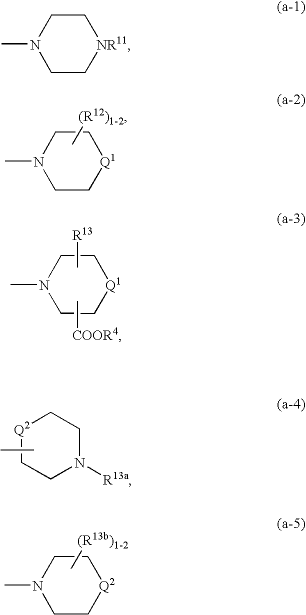
wherein
each Q¹ independently is a direct bond, —CH₂—, or —CH₂—CH₂—;
each Q² independently is O, S, SO or SO₂;
each R⁴ independently is hydrogen, C₁₋₄alkyl, arylC₁₋₄alkyl;
each R^{5a}, R^{5b}, R^{5c}, R^{5d} independently is hydrogen, C₁₋₄alkyl or arylC₁₋₄alkyl;
each R^{5e}, R^{5f} independently is hydrogen, C₁₋₄alkyl or arylC₁₋₄alkyl, or R^{5e} and R^{5f}, taken together may form a bivalent alkanediyl radical of formula —CH₂—CH₂— or —CH₂—CH₂—CH₂—;
R⁶ is C₁₋₄alkyl, —N(R^{5a}R^{5b}), C₁₋₄alkyloxy, pyrrolidin-1-yl, piperidin-1-yl, homopiperidin-1-yl, piperazin-1-yl, 4-(C₁₋₄alkyl)-piperazin-1-yl, morpholin-4-yl-, thiomorpholin-4-yl-, 1-oxothiomorpholin-4-yl and 1,1-dioxo-thiomorpholin-4-yl;
R⁷ is hydrogen, C₁₋₄alkyl, hydroxyC₁₋₄alkyl, C₁₋₄alkoxyC₁₋₄alkyl or C₁₋₄alkylcarbonyloxyC₁₋₄alkyl;
R⁸ is hydroxyC₁₋₄alkyl, C₁₋₄alkoxyC₁₋₄alkyl, C₁₋₄alkylcarbonyloxyC₁₋₄alkyl, aryl or arylC₁₋₄alkyl;
R⁹ is hydrogen or C₁₋₄alkyl;
R¹⁰ is Het₁, Het₂ or a radical 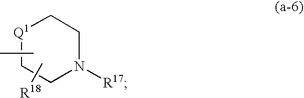
R¹¹ is aryl, arylC₁₋₄alkyl, formyl, C₁₋₄alkylcarbonyl, arylcarbonyl, arylC₁₋₄alkylcarbonyl, C₁₋₄alkyloxycarbonyl, arylC₁₋₄alkyloxycarbonyl, R^{5a}R^{5b}N-carbonyl, hydroxyC₁₋₄alkyl, C₁₋₄alkyloxyC₁₋₄alkyl, arylC₁₋₄alkyloxyC₁₋₄alkyl, aryloxyC₁₋₄alkyl, Het₂;
each R¹² independently is hydroxy, C₁₋₄alkyl, arylC₁₋₄alkyl, C₁₋₄alkyloxy, arylC₁₋₄alkyloxy, oxo, spiro(C₂₋₄alkanedioxy), spiro(diC₁₋₄alkyloxy), —NR^{5a}R^{5b};
R¹³ is hydrogen, hydroxy, C₁₋₄alkyl, C₁₋₄alkyloxy, or arylC₁₋₄alkyloxy; or
R^{13a} is C₁₋₄alkyl, arylC₁₋₄alkyl, C₁₋₄alkyloxycarbonyl or arylC₁₋₄alkyloxycarbonyl;
each R^{13b} is hydrogen or C₁₋₄alkyl; or R² is
iv) a radical of formula: 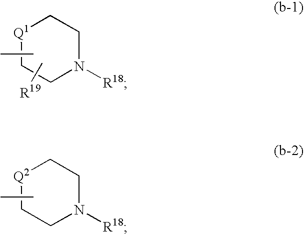
—CₚH₂ₚ—CH(OR¹⁴)—C_{q}H_{2q}—R¹⁵ (b-3);
—CH₂—CH₂—(O—CH₂—CH₂)ₘ—OR¹⁴ (b-4);
—CH₂—CH₂—(O—CH₂—CH₂)ₘ—NR^{17a}R^{17b} (b-5);
wherein in radical (b-3) one of the hydrogen atoms in —CₚH₂ₚ— and one of the hydrogen atoms in —CH(OR¹⁴)—C_{q}H_{2q}—, that is not part of R¹⁴, may be replaced by a direct bond or a C₁₋₄alkanediyl group;
p is 1, 2 or 3;
q is 0, 1, 2 or 3;
each m independently is 1 to 10;
each R¹⁴ independently is hydrogen, C₁₋₄alkyl, aryl C₁₋₄alkyl, aryl, C₁₋₄alkylcarbonyl, —SO₃H, —PO₃H₂;
R¹⁵ is a substituent selected from the group consisting of cyano, NR^{16a}R^{16b}, pyrrolidinyl, piperidinyl, homopiperidinyl, piperazinyl, 4-(C₁₋₄alkyl)-piperazinyl, 4-(C₁₋₄alkylcarbonyl)-piperazinyl, 4-(C₁₋₄alkyloxycarbonyl)-piperazinyl, morpholinyl, thiomorpholinyl, 1-oxothiomorpholinyl, 1,1-dioxo-thiomorpholinyl, aryl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, pyridyl, pyrimidinyl, pyrazinyl, pyridazinyl, triazinyl, hydroxy-carbonyl, C₁₋₄alkylcarbonyl, N(R^{16a}R^{16b})carbonyl, C₁₋₄alkyloxycarbonyl, pyrrolidin-1-ylcarbonyl, piperidin-1-ylcarbonyl, homopiperidin-1-ylcarbonyl, piperazin-1-ylcarbonyl, 4-(C₁₋₄alkyl)-piperazin-1-ylcarbonyl, morpholin-1-yl-carbonyl, thiomorpholin-1-yl-carbonyl, 1-oxothiomorpholin-1-ylcarbonyl and 1,1-dioxo-thiomorpholin-1-ylcarbonyl; or R¹⁵ may additionally be aryl substituted with a radical —COOR⁴; or a radical selected from —NR^{5a}—C(═NR^{5b})—NR^{5c}R^{5d}, —NR^{5a}—C(═NR^{5e})—R^{5f}, —O—NR^{5a}—C(═NR^{5b})—NR^{5c}R^{5d}, —O—NR^{5a}—C(═NR^{5e})—R^{5f}, -sulfonyl-R⁶, —NR⁷R⁸, —NR⁹R¹⁰, a radical (a-1), (a-2), (a-3), (a-4) or (a-5); wherein R⁴, R^{5a}, R^{5b}, R^{5c}, R^{5d}, R⁶, R⁷, R⁸, R⁹, R¹⁰, and the radicals (a-1), (a-2), (a-3), (a-4), (a-5) independently are as defined above;
R^{16a} and R^{16b} independently from one another are hydrogen, C₁₋₆alkyl or C₁₋₆alkyl substituted with a substituent selected from the group consisting of amino, mono- or di(C₁₋₄alkyl)amino, pyrrolidinyl, piperidinyl, homopiperidinyl, piperazinyl, 4-(C₁₋₄alkyl)-piperazinyl, morpholinyl, thiomorpholinyl, 1-oxothiomorpholinyl, 1,1-dioxo-thiomorpholinyl and aryl;
R^{17a} and R^{17b} independently from one another are hydrogen, C₁₋₄alkyl or arylC₁₋₄alkyl;
or R^{17a} and R^{17b} together with the nitrogen atom to which they are attached form a pyrrolidinyl, piperidinyl, homopiperidinyl, morpholinyl, thiomorpholinyl, 1-oxothiomorpholinyl, 1,1-dioxo-thiomorpholinyl, piperazinyl, 4-C₁₋₄alkyl-piperazinyl, 4-(C₁₋₄alkylcarbonyl)-piperazinyl, 4-(C₁₋₄alkyloxycarbonyl)-piperazinyl ring;
each R¹⁸ independently is hydrogen, C₁₋₄alkyl, arylC₁₋₄alkyl, C₁₋₄alkylcarbonyl or C₁₋₄alkyloxycarbonyl;
R¹⁹ is hydrogen, hydroxy, C₁₋₄alkyl or a radical —COOR⁴;
R³ is nitro, cyano, amino, halo, hydroxy, C₁₋₄alkyloxy, hydroxycarbonyl, aminocarbonyl, C₁₋₄alkyloxycarbonyl, mono- or di(C₁₋₄alkyl)aminocarbonyl, C₁₋₄alkylcarbonyl, methanimidamidyl, mono- or di(C₁₋₄alkyl)methanimidamidyl, N-hydroxy-methanimidamidyl or Het₁;
aryl is phenyl optionally substituted with one or more substituents each individually selected from the group consisting of C₁₋₆alkyl, C₁₋₄alkoxy, halo, hydroxy, amino, trifluoromethyl, cyano, nitro, hydroxyC₁₋₆alkyl, cyanoC₁₋₆alkyl, mono- or di(C₁₋₄alkyl)amino, aminoC₁₋₄alkyl, mono- or di(C₁₋₄alkyl)aminoC₁₋₄alkyl;
Het₁ is a 5-membered ring system wherein one, two, three or four ring members are heteroatoms each individually and independently selected from the group consisting of nitrogen, oxygen and sulfur, and wherein the remaining ring members are carbon atoms; and, where possible, any nitrogen ring member may optionally be substituted with C₁₋₄alkyl; any ring carbon atom may, each individually and independently, optionally be substituted with a substituent selected from the group consisting of C₁₋₄alkyl, C₂₋₆alkenyl, C₃₋₇cycloalkyl, hydroxy, C₁₋₄alkoxy, halo, amino, cyano, trifluoromethyl, hydroxyC₁₋₄alkyl, cyanoC₁₋₄alkyl, mono- or di(C₁₋₄alkyl)amino, aminoC₁₋₄alkyl, mono- or di(C₁₋₄alkyl)aminoC₁₋₄alkyl, arylC₁₋₄alkyl, aminoC₂₋₆alkenyl, mono- or di(C₁₋₄alkyl)aminoC₂₋₆alkenyl, furanyl, thienyl, pyrrolyl, oxazolyl, thiazolyl, imidazolyl, isoxazolyl, isothiazolyl, pyrazolyl, oxadiazolyl, thiadiazolyl, triazolyl, tetrazolyl, aryl, hydroxycarbonyl, aminocarbonyl, C₁₋₄alkyloxycarbonyl, mono- or di(C₁₋₄alkyl)aminocarbonyl, C₁₋₄alkylcarbonyl, oxo, thio; and wherein any of the foregoing furanyl, thienyl, pyrrolyl, oxazolyl, thiazolyl, imidazolyl, isoxazolyl, isothiazolyl, pyrazolyl, oxadiazolyl, thiadiazolyl and triazolyl moieties may optionally be substituted with C₁₋₄alkyl;
Het₂ is pyridyl, pyrimidinyl, pyrazinyl, pyridazinyl or triazinyl, wherein any ring carbon atom of each of said 6-membered nitrogen containing aromatic rings may optionally be substituted with C₁₋₄alkyl.

2. A compound according to claim 1 wherein n is 1 or 2;

3. A compound according to claim 1 wherein R¹ is hydrogen, cyano, halo, aminocarbonyl, C₁₋₄alkylaminocarbonyl, hydroxycarbonyl, C₁₋₄alkyloxycarbonyl, arylaminocarbonyl, N-hydroxy-methanimidamidyl, mono- or di(C₁₋₄alkyl)-methanimidamidyl, Het₁ or Het₂.

4. A compound according to claim 1 wherein R¹ is cyano.

5. A compound according to claim 1 wherein X is O;
X is NR² wherein R² is C₁₋₁₀alkyl being substituted with aryl, wherein said aryl is substituted with a radical —COOR⁴;
X is NR² wherein R² is C₁₋₁₀alkyl substituted with a radical selected from —NR^{5a}—C(═NR^{5b})—NR^{5c}R^{5d}, —O—NR^{5a}—C(═NR^{5b})—NR^{5c}R^{5d}, -sulfonyl-R⁶, —NR⁷R⁸, —NR⁹R¹⁰, a radical (a-1), (a-2), (a-3), (a-4) and (a-5);
X is NR² wherein R² is a radical, wherein R¹⁹ is hydrogen or —COOR⁴ and wherein Q¹ in radical is a direct bond or —CH₂—;
X is NR² wherein R² is a radical, wherein Q² is O;
X is NR² wherein R² is a radical wherein q is 1, 2 or 3;
X is NR² wherein R² is a radical wherein m is 1-6; or
X is NR² wherein R² is a radical wherein m is 1-5.

6. A compound according to claim 1 wherein X is NR² wherein R² is a radical wherein q is 1, 2 or 3.

7. A compound according to claim 1 wherein X is NR² wherein R² is a radical R¹⁵ is NR^{16a}R^{16b}, pyrrolidinyl, piperidinyl, 4-morpholinyl.

8. A compound according to claim 1 wherein
R³ is nitro, cyano, halo, C₁₋₄alkyloxy, hydroxycarbonyl, aminocarbonyl, mono- or di(C₁₋₄alkyl)methanimidamidyl, N-hydroxy-methanimidamidyl, oxadiazolyl, isoxazolyl, thiazolyl, furanyl, isoxazolyl, tetrazolyl, wherein each of said oxadiazolyl, isoxazolyl, thiazolyl, furanyl, isoxazolyl, tetrazolyl may optionally be substituted with C₁₋₄alkyl, hydroxy, cyano, trifluoromethyl.

9. A compound according to claim 1 wherein R³ is nitro.

10. A compound selected from the group consisting of
1-(4-Nitro-phenyl)-2-oxo-1,2-dihydro-benzo[4,5]furo[3,2-b]pyridine-3-carbonitrile,
5-(2-Hydroxy-3-piperidin-1-yl-propyl)-1-(4-nitro-phenyl)-2-oxo-2,5-dihydro-1H-pyrido[3,2-b]indole-3-carbonitrile,
5-(3-Diethylamino-2-hydroxy-propyl)-1-(4-nitro-phenyl)-2-oxo-2,5-dihydro-1H-pyrido[3,2-b]indole-3-carbonitrile,
5-[2-(2-Methoxy-ethoxy)-ethyl]-1-(4-nitro-phenyl)-2-oxo-2,5-dihydro-1H-pyrido[3,2-b]indole-3-carbonitrile, and especially
5-(2-Hydroxy-3-pyrrolidin-1-yl-propyl)-1-(4-nitro-phenyl)-2-oxo-2,5-dihydro-1H-pyrido[3,2-b]indole-3-carbonitrile, and especially
5-(2-Hydroxy-3-morpholin-4-yl-propyl)-1-(4-nitro-phenyl)-2-oxo-2,5-dihydro-1H-pyrido[3,2-b]indole-3-carbonitrile,

11. (canceled)

12. A pharmaceutical composition, comprising an effective amount of at least one compound of formula (I) as defined in claim 1 and a pharmaceutically tolerable excipient.

13. A process for preparing a compound as claimed in claim 1, comprising
(a) converting an intermediate (II) using a N-alkylating agent to obtain a compound of formula (I-b): 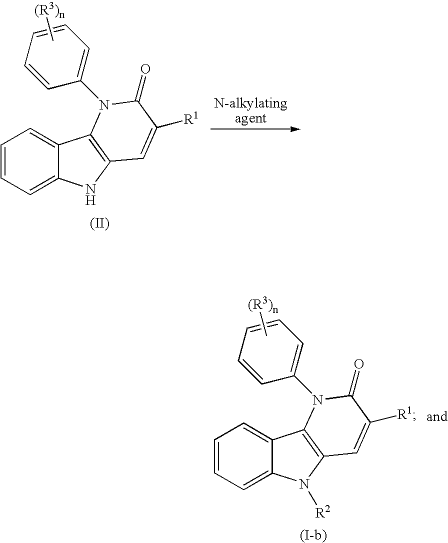
(b) cyclizing an intermediate (VI-d) to obtain compounds of formula (I-e):

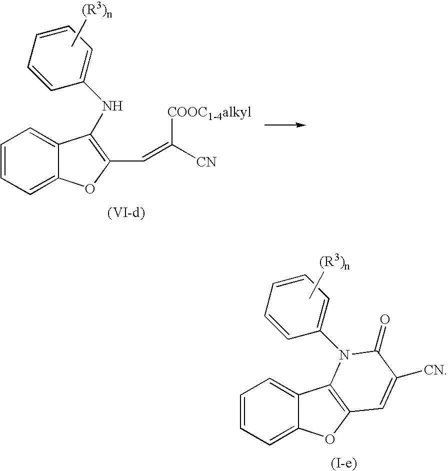

14. A compound of formula (IV-a): 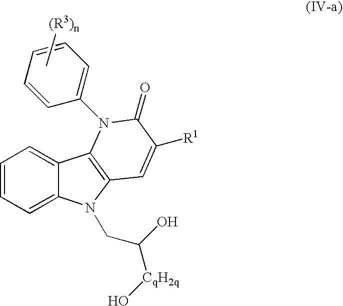
wherein q, R¹, R³ and n are as defined in claim 1, or a salt or possible stereochemically isomeric form thereof.

15. A compound of formula (IV-b): 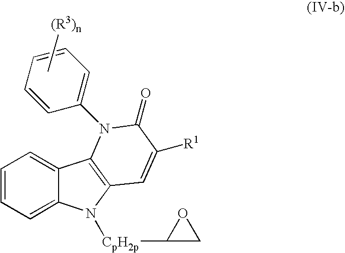
wherein p, R¹, R³ and n are as defined in claim 1, or a salt or possible stereochemically isomeric form thereof.

16. A compound of formula (V): 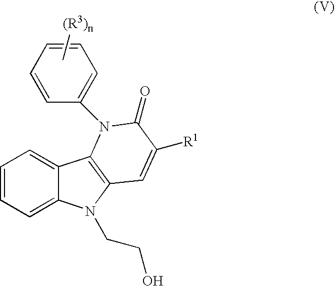
wherein R¹, R³ and n are as defined in claim 1, or a salt or possible stereochemically isomeric form thereof.

17. A pharmaceutical composition, comprising an effective amount of at least one compound of formula (I) as defined in claim 2 and a pharmaceutically tolerable excipient.

18. A pharmaceutical composition, comprising an effective amount of at least one compound of formula (I) as defined in claim 3 and a pharmaceutically tolerable excipient.

19. A pharmaceutical composition, comprising an effective amount of at least one compound of formula (I) as defined in claim 4 and a pharmaceutically tolerable excipient.

20. A pharmaceutical composition, comprising an effective amount of at least one compound of formula (I) as defined in claim 5 and a pharmaceutically tolerable excipient.

21. A pharmaceutical composition, comprising an effective amount of at least one compound of formula (I) as defined in claim 6 and a pharmaceutically tolerable excipient.

22. A pharmaceutical composition, comprising an effective amount of at least one compound of formula (I) as defined in claim 7 and a pharmaceutically tolerable excipient.

23. A pharmaceutical composition, comprising an effective amount of at least one compound of formula (I) as defined in claim 8 and a pharmaceutically tolerable excipient.

24. A pharmaceutical composition, comprising an effective amount of at least one compound of formula (I) as defined in claim 9 and a pharmaceutically tolerable excipient.

25. A pharmaceutical composition, comprising an effective amount of at least one compound of formula (I) as defined in claim 10 and a pharmaceutically tolerable excipient.
